(19) United States
(12) Patent Application Publication
(10) Pub. No.: US 2010/0168175 A1
(43) Pub. Date: Jul. 1, 2010

(54) ATPENINS

(75) Inventors: Isabelle ADELT, Haan (DE); Robert VELTEN, Langenfeld (DE); Welf Burkhard WIESE, Langenfeld (DE); Ulrike WACHENDORFF-NEUMANN, Neuwied (DE); Karl-Heinz KUCK, Langenfeld (DE); Peter DAHMEN, Neuss (DE); Arnd VOERSTE, Köln (DE); Ronald EBBERT, Nürnberg (DE); Franz von NUSSBAUM, Düsseldorf (DE)

Correspondence Address: Baker Donelson Bearman, Caldwell & Berkowitz, PC, 555 Eleventh Street, NW, Sixth Floor, Washington, DC, 20004, US

(73) Assignee: BAYER CROPSCIENCE AG, Monheim (DE)

(21) Appl. No.: 12/635,444

(22) Filed: Dec. 10, 2009

(30) Foreign Application Priority Data
Dec. 18, 2008 (EP) 08172151.6

(51) Int. Cl. A01N 43/40; A01N 43/80; C07D 401/06; C07D 413/06; C07D 409/06; C07D 405/10; C07D 213/69; C07D 213/82; C07D 213/79; A01P 1/00

(52) U.S. Cl. 514/335; 514/336; 514/340; 514/348; 546/261; 546/272.1; 546/280.4; 546/283.7; 546/296

(57) ABSTRACT

The present invention relates to processes for preparing certain 2-pyridones and 2-pyridinols, to novel compounds of these two types and to their use as biologically active compounds, in particular for controlling harmful microorganisms in crop protection, in the medicinal field and in the protection of materials.

The present invention relates to processes for preparing certain 2-pyridones and 2-pyridinols, to novel compounds of these two types and to their use as biologically active compounds, in particular for controlling harmful microorganisms in crop protection, in the medicinal field and in the protection of materials.

Certain substituted 2-pyridones and 2-pyridinols and their biological action, for example as fungicides or as insecticides, are already known. The preparation of certain 2-pyridones and 2-pyridinols by fermentation and, if appropriate, by subsequent derivatization has also been described [see, for example, J. Antibiotics 1988, 41, 1769-1773, ibid. 1990, 43, 1064-1068, ibid. 1990, 43, 1553-1558, ibid. 1992, 45, 1970-1973, Pestic. Sci. 1989, 27, 155-164, J. Chem. Soc. Perkin Trans. I 1989, 1885-1887, Agric. Biol. Chem. 1991, 55, 2629-2631, EP-A 1 512 402 (WO 03/103667), JP-A 4-224559].

The application of these processes has the disadvantage that they only afford those 2-pyridones or 2-pyridinols which are produced by the fermentation strain used or can be obtained from the fermentation products by further derivatization.

Also known is the synthesis of certain 2-pyridones and 2-pyridinols (c.f. J. Org. Chem. 1994, 59, 6173-6178 and J. Heterocyclic Chem. 1995, 32, 1117-1124, WO 2006/137389, K. A. Hughes' poster “Synthethic Atpenin Analogs: Potent Mitochondrial Inhibitors of Fungal & Mammalian Succinate-ubiquinone Oxidoreductase” at the 236th ACS Meeting 2008). Here, starting with 2-chloropyridine, the substituents at the heterocycle are introduced in a linear sequence by step-wise functionalization. The use of this process has the disadvantages of the multi-step synthesis, the fact that a plurality of metallization reactions have to be carried out and the low overall yield. Thus, for example, the described synthesis of the pentasubstituted pyridine of atpenin B requires four metallization reactions and one metal-halogen exchange reaction and affords the product in 13 linear steps in an overall yield of only 2.6%.

Accordingly, it was surprising that certain 2-pyridones and 2-pyridinols can also be obtained in one reaction step by condensation of suitable reaction partners.

The present invention now provides processes allowing the synthesis of 2-pyridones of the general formula (I)

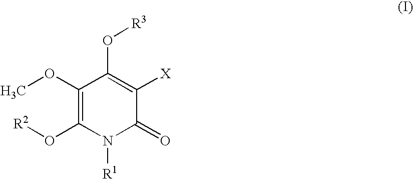

and of 2-pyridinols of the general formula (II)

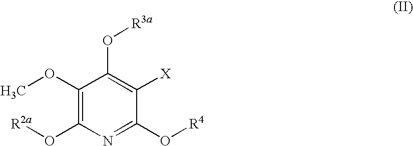

in which
X represents hydrogen, —C(═O)OR⁵, —C(═O)NR⁶R⁷, —C(═O)CR⁸R⁹R¹⁰, or represents —C(═O)aryl or —(C═O)hetaryl, each of which is optionally substituted in the aryl or heteraryl moiety,
R^{l} represents hydrogen or alkyl, alkenyl, alkynyl, cycloalkyl, aryl, hetaryl, optionally mono- or polysubstituted by identical or different substituents,
R² represents hydrogen, methyl or ethyl,
R^{2a} represents methyl or ethyl,
R³ represents hydrogen,
R^{3a} and R⁴ both represent hydrogen or both represent acetyl,
R⁵ represents hydrogen or alkyl, alkenyl, alkynyl, cycloalkyl, cycloalkenyl, bicycloalkyl, aryl, hetaryl, optionally mono- or polysubstituted by identical or different substituents,
R⁶ represents hydrogen, C₁-C₈-alkyl, C₁-C₄-alkoxy-C₁-C₄-alkyl, C₃-C₈-cycloalkyl; C₂-C₆-haloalkyl, halo-C₂-C₄-alkoxy-C₁-C₄-alkyl, C₃-C₈-halocycloalkyl having in each case 1 to 9 fluorine, chlorine and/or bromine atoms; (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₂-C₃-alkoxy)carbonyl-C₁-C₃-alkyl having in each case 1 to 13 fluorine, chlorine and/or bromine atoms; represents —CH₂—C≡C—R¹¹ or —CH₂—CH═CH—R¹¹,
R⁷ represents hydrogen or represents alkyl, alkenyl, alkynyl, cycloalkylalkyl, cycloalkenylalkyl, each of which is optionally mono- or polysubstituted by identical or different substituents, or represents -M-Q-Z,
R⁸ represents hydrogen, alkyl or haloalkyl,
R⁹ represents hydrogen, halogen or represents alkyl, alkenyl, alkynyl, alkoxy, alkylthio, cycloalkyl, cycloalkenyl, or bicycloalkyl, each of which is optionally mono- or polysubstituted by identical or different substituents,
R¹⁰ represents hydrogen or represents alkyl, alkenyl, alkynyl, cycloalkyl, cycloalkylalkyl, cycloalkenyl, cycloalkenylalkyl, bicycloalkyl, bicycloalkylalkyl, aryl, arylalkyl, hetaryl or hetarylalkyl, each of which is optionally mono- or polysubstituted by identical or different substituents,
R⁹ and R¹⁰ furthermore together with the carbon atom to which they are attached form a carbocycle or heterocycle which may be saturated or unsaturated and which may be fused with a further carbocycle and which may furthermore optionally be substituted,
R¹¹ represents hydrogen, C₁-C₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)carbonyl or cyano,
M represents in each case optionally substituted cycloalkylene, cycloalkenylene, bicycloalkylene, arylene or hetarylene,
Q represents a direct bond, C₁-C₄-alkylene, C₂-C₄-alkenylene, C₁-C₄-alkylenoxy, oxy-C₁-C₄-alkylene, O, S, SO, SO₂ or NR¹²,
Z represents hydrogen or represents Z¹, Z², Z³, Z⁴, Z⁵ or Z⁶, where Q does not represent O, S, SO, SO₂, NR¹² if Z represents hydrogen,
Z¹ represents phenyl which is optionally mono- to pentasubstituted by identical or different substituents,
Z² represents pyridinyl which is optionally mono- to trisubstituted by identical or different substituents,
Z³ represents cycloalkyl or bicycloalkyl, each of which is optionally mono- or polysubstituted by identical or different substituents from the group consisting of halogen, alkyl, cycloalkyl and/or —(CR¹³R¹⁴)ₘSiR¹⁵R¹⁶R¹⁷,
Z⁴ represents unsubstituted C₁-C₂₀-alkyl or represents C₁-C₂₀-alkyl which is mono- or polysubstituted by identical or different substituents from the group consisting of halogen, alkylthio, alkylsulphynyl, alkylsulphonyl, alkoxy, alkylamino, dialkylamino, haloalkylthio, haloalkylsulphynyl, haloalkylsulphonyl, haloalkoxy, haloalkylamino, halodialkylamino, —SiR¹⁵R¹⁶R¹⁷ and C₃-C₆-cycloalkyl, where the cycloalkyl moiety for its part may optionally be mono- or polysubstituted by identical or different substituents from the group consisting of halogen and C₁-C₄-alkyl,
Z⁵ represents C₂-C₂₀-alkenyl or C₂-C₂₀-alkynyl, each of which is optionally mono- or polysubstituted by identical or different substituents from the group consisting of halogen, alkylthio, alkylsulphynyl, alkylsulphonyl, alkoxy, alkylamino, dialkylamino, haloalkylthio, haloalkylsulphynyl, haloalkylsulphonyl, haloalkoxy, haloalkylamino, halodialkylamino, —SiR¹⁵R¹⁶R¹⁷ and/or C₃-C₆-cycloalkyl, where the cycloalkyl moiety for its part may optionally be mono- or polysubstituted by identical or different substituents from the group consisting of halogen and C₁-C₄-alkyl,
Z⁶ represents a saturated or unsaturated 3- to 7-membered ring which is optionally mono- or polysubstituted and which contains a silicon atom as ring member, in which Q represents a direct bond or C₁-C₄-alkylene,

or

M-Q-Z together represent 1H-2,3-dihydroinden-4-yl, 1,3-dihydro-2-benzofuran-4-yl or 1,3-dihydro-2-benzothien-4-yl, each of which is optionally mono- to trisubstituted by methyl, or represent 1,2,3,4-tetrahydro-9-isopropyl-1,4-methanonaphthalen-5-yl,
R¹² represents hydrogen, C₁-C₈-alkyl, C₁-C₄-alkoxy-C₁-C₄-alkyl, C₁-C₄-alkylthio-C₁-C₄-alkyl, C₃-C₈-alkenyl, C₃-C₈-alkynyl, C₂-C₆-haloalkyl, C₃-C₆-haloalkenyl, C₃-C₆-haloalkynyl or C₃-C₆-cycloalkyl,
R¹³ represents hydrogen or C₁-C₄-alkyl,
R¹⁴ represents hydrogen or C₁-C₄-alkyl,
m represents 0, 1, 2 or 3,
R¹⁵ and R¹⁶ independently of one another represent hydrogen, C₁-C₈-alkyl, C₁-C₈-alkoxy, C₁-C₄-alkoxy-C₁-C₄-alkyl, C₁-C₄-alkylthio-C₁-C₄-alkyl or C₁-C₆-haloalkyl,
R¹⁷ represents hydrogen, C₁-C₈-alkyl, C₁-C₈-alkoxy, C₁-C₄-alkoxy-C₁-C₄-alkyl, C₁-C₄-alkylthio-C₁-C₄ alkyl, C₂-C₈-alkenyl, C₂-C₈-alkynyl, C₁-C₆-haloalkyl, C₂-C₆-haloalkenyl, C₂-C₆-halo-alkynyl, C₃-C₆-cycloalkyl, or represents in each case optionally substituted phenyl or phenylalkyl,

from readily accessible starting materials in high chemical variability and in a simple manner.

According to the invention, the 2-pyridones of the formula (I) and the 2-pyridinols of the formula (II) can be present as mixtures of various possible isomeric forms, in particular of stereoisomers, such as, for example, E- and Z—, threo and erythro, and also optical isomers, and, if appropriate, also of tautomers. By the processes according to the invention, it is possible to obtain both the E and the Z isomers, as well as the threo and erythro and also the optical isomers, any mixtures of these isomers, and also the possible tautomeric forms.

The compounds of the formulae (I) and (II) prepared are preferably those in which the individual radicals have the following meanings:
X preferably represents hydrogen, —C(═O)OR⁵, —C(═O)NR⁶R⁷, —C(═O)CR⁸R⁹R¹⁰, represents —C(═O)aryl or —(C═O)hetaryl which may in each case be mono- or polysubstituted in the aryl moiety or hetaryl moiety by identical or different substituents from the group consisting of halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₃-C₆-cycloalkyl, C₁-C₂-alkylenedioxy, C₁-C₂-haloalkylenedioxy, aryl, aryloxy, hetaryl, hetaryloxy, where the latter aryl, aryloxy, hetaryl and hetaryloxy substituents for their part may be substituted by halogen, cyano, C₁-C₄-haloalkyl, C₁-C₄-alkoxy, C₁-C₄-haloalkoxy, C₁-C₄-alkylthio or C₁-C₄-haloalkylthio.
X particularly preferably represents hydrogen, —C(═O)OR⁵, —C(═O)NHR⁷, —C(═O)NR⁶R⁷, —C(═O)CR —C(═O)aryl which may optionally be mono- to tetrasubstituted in the aryl moiety by identical or different substituents from the group consisting of fluorine, chlorine, bromine, cyano, methyl, ethyl, n-, i-propyl, n-, s-, t-butyl, trifluoromethyl, methoxy, ethoxy, n-, i-propoxy, n-, s-, t-butoxy, trifluoromethoxy, cyclopropyl, cyclohexyl, methylenedioxy, ethylenedioxy, difluoromethylenedioxy, tetrafluoroethylenedioxy, phenyl or phenoxy, where the latter phenyl and phenoxy substituents for their part may be substituted by fluorine, chlorine, bromine, cyano, methyl, trifluoromethyl, methoxy or trifluoromethoxy.
X very particular preferably represents hydrogen.
X very particularly preferably represents —C(═O)OR⁵.
X very particularly preferably represents —C(═O)NHR⁷ or —C(═O)NR⁶R⁷.
X very particularly preferably represents —C(═O)CR⁸R⁹R¹⁰.
X very particularly preferably represents phenylcarbonyl or naphthylcarbonyl which may optionally be mono- to trisubstituted by identical or different substituents from the group consisting of fluorine, chlorine, cyano, methyl, i-propyl, t-butyl, trifluoromethyl, methoxy, trifluoromethoxy, cyclohexyl, methylenedioxy, phenyl and phenoxy, where the latter phenyl and phenoxy substituents for their part may be substituted by fluorine, chlorine, cyano, trifluoromethyl or trifluoromethoxy.
R¹ preferably represents hydrogen, C₁-C₄-alkyl, allyl, propargyl, C₃-C₆-cycloalkyl or represents arylmethyl which is mono- or polysubstituted by identical or different substituents from the group consisting of halogen, C₁-C₄-alkyl, C₁-C₄-haloalkyl, C₁-C₄-alkoxy and C₁-C₄-haloalkoxy.
R¹ particularly preferably represents hydrogen, methyl, n-propyl, i-propyl, allyl, cyclopropyl, cyclohexyl or represents benzyl which is optionally mono- to tetrasubstituted by identical or different substituents from the group consisting of fluorine, chlorine, methyl, trifluoromethyl, and methoxy.
R¹ very particularly preferably represents hydrogen, methyl, n-propyl, allyl or represents benzyl which is optionally mono- or disubstituted by methoxy.
R² preferably represents hydrogen.
R² furthermore preferably represents methyl.
R² furthermore preferably represents ethyl.
R² particularly preferably represents methyl.
R^{2a} preferably represents methyl.
R^{2a} furthermore preferably represents ethyl.
R^{2a} particularly preferably represents methyl.
R^{3a} and R⁴ preferably both simultaneously represent hydrogen.
R^{3a} and R⁴ furthermore preferably both simultaneously represent acetyl.
R^{3a} and R⁴ particularly preferably both simultaneously represent hydrogen.
R⁵ preferably represents hydrogen, C₁-C₄-alkyl, allyl, propargyl, C₃-C₆-cycloalkyl or represents arylmethyl which is optionally mono- or polysubstituted by identical or different substituents from the group consisting of halogen, C₁-C₄-alkyl, C₁-C₄-haloalkyl and C₁-C₄-alkoxy.
R⁵ particularly preferably represents hydrogen, methyl, ethyl, n-propyl or benzyl.
R⁵ very particularly preferably represents hydrogen or methyl.
R⁶ preferably represents hydrogen, C₁-C₆-alkyl, C₁-C₃-alkoxy-C₁-C₃-alkyl, C₃-C₆-cycloalkyl; C₂-C₄-haloalkyl, halo-C₂-C₃-alkoxy-C₁-C₃-alkyl, C₃-C₈-halocycloalkyl having in each case 1 to 9 fluorine, chlorine and/or bromine atoms; (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₂-C₃-alkoxy)carbonyl-C₁-C₃-alkyl having in each case 1 to 13 fluorine, chlorine and/or bromine atoms; represents —CH₂—C≡C—R¹¹ or —CH₂—CH═CH—R¹¹.
R⁶ particularly preferably represents hydrogen, methyl, ethyl, n- or isopropyl, n-, iso-, sec- or tert-butyl, pentyl or hexyl, methoxyethyl, ethoxyethyl, cyclopropyl, cyclopentyl, cyclohexyl, difluoroethyl, trifluoroethyl, —CH₂—CO—CH₃, —CH₂—CO—CH₂CH₃, —CH₂—CO—CH(CH₃)₂, —(CH₂)₂—CO—CH₃, —(CH₂)₂—CO—CH₂CH₃, —(CH₂)₂—CO—CH(CH₃)₂, —CH₂—CO₂CH₃, —CH₂—CO₂CH₂CH₃, —CH₂—CO₂CH(CH₃)₂, —(CH₂)₂—CO₂CH₃, —(CH₂)₂—CO₂CH₂CH₃, —(CH₂)₂—CO₂CH(CH₃)₂, —CH₂—CO—CF₃, —CH₂—CO—CC₃, —CH₂—CO—CH₂CF₃, —CH₂—CO—CH₂CCl₃, —(CH₂)₂—CO—CH₂CF₃, —(CH₂)₂—CO—CH₂CCl₃, —CH₂—CO₂CH₂CF₃, —CH₂—CO₂CF₂CF₃, —CH₂—CO₂CH₂CCl₃, —CH₂—CO₂CCl₂CCl₃, —(CH₂)₂—CO₂CH₂CF₃, —(CH₂)₂—CO₂CF₂CF₃, —(CH₂)₂—CO₂CH₂CCl₃, —(CH₂)₂—CO₂CCl₂CCl₃; represents —CH₂—C≡C—R¹¹ or —CH₂—CH≡CH—R¹¹.
R⁶ very particularly preferably represents hydrogen, methyl, cyclopropyl, —CH₂—C≡CH or —CH₂—CH═CH₂.
R⁶ especially preferably represents hydrogen or methyl.
R⁷ preferably represents hydrogen or C₁-C₆-alkyl which is optionally mono- or polysubstituted by identical or different phenyl which may optionally be substituted, or represents -M-Q-Z.
R⁷ particularly preferably represents in each case optionally phenyl-substituted methyl, propyl (in particular 2-phenylpropyl), n-butyl or pentyl (in particular 2-methylbutyl) or represents -M-Q-Z.
R⁸ preferably represents hydrogen or C₁-C₈-alkyl.
R⁸ particularly preferably represents hydrogen or C₁-C₄-alkyl.
R⁸ very particularly preferably represents hydrogen or methyl.
R⁹ preferably represents hydrogen, C₁-C₄-alkyl, C₁-C₄-alkoxy, C₁-C₄-alkylthio or C₃-C₆-cycloalkyl.
R⁹ particularly preferably represents hydrogen, methyl, ethyl, n-, i-propyl, n-, s-butyl, methoxy, ethoxy, n-, i-propoxy, n-, s-, t-butoxy, methylthio, ethylthio, n-, i-propylthio, n-, s-, t-butylthio or cyclopropyl.
R⁹ very particularly preferably represents hydrogen, methyl, ethyl, n-propyl or n-butyl.
R¹⁰ preferably represents hydrogen,
   represents in each case straight-chain or branched C₁-C₁₂-alkyl, C₂-C₁₂-alkenyl or C₂-C₁₂-alkynyl, each of which is optionally mono- or polysubstituted by identical or different substituents from the group consisting of halogen, C₁-C₄-alkoxy, C₁-C₄-alkoxycarbonyl, C₃-C₇-cycloalkyl (which for its part may be substituted by halogen, C₁-C₄-alkyl or C₁-C₄-alkoxy), phenyl, benzyl, phenyloxy, benzyloxy (which for their part may in each case be substituted by halogen, cyano, nitro, C₁-C₄-alkyl, C₁-C₄-haloalkyl, C₁-C₄-haloalkoxy having in each case 1 to 9 fluorine, chlorine and/or bromine atoms, phenyl, phenoxy, hetaryl or hetaryloxy, where the latter phenyl, phenoxy, hetaryl or hetaryloxy substituents for their part may optionally be substituted by halogen, cyano, nitro, C₁-C₄-alkyl, C₁-C₄-haloalkyl, C₁-C₄-haloalkoxy having in each case 1 to 9 fluorine, chlorine and/or bromine atoms) and hetaryl (which for its part may be substituted by halogen, cyano, nitro, C₁-C₄-alkyl, C₁-C₄-haloalkyl, C₁-C₄-haloalkoxy having in each case 1 to 9 fluorine, chlorine and/or bromine atoms, phenyl, phenoxy, hetaryl or hetaryloxy, where the latter phenyl, phenoxy, hetaryl or hetaryloxy substituents for their part may be substituted by halogen, cyano, nitro, C₁-C₄-alkyl, C₁-C₄-haloalkyl, C₁-C₄-haloalkoxy having in each case 1 to 9 fluorine, chlorine and/or bromine atoms),
   represents cycloalkyl, cycloalkenyl, phenyl or hetaryl, optionally mono- or polysubstituted by identical or different substituents from the group consisting of halogen, C₁-C₄-alkyl, C₁-C₄-haloalkyl having 1 to 9 fluorine, chlorine and/or bromine atoms, phenoxy (which for its part may be substituted by halogen, cyano, nitro, C₁-C₄-alkyl, C₁-C₄-haloalkyl, C₁-C₄-haloalkoxy having in each case 1 to 9 fluorine, chlorine and/or bromine atoms).
R¹⁰ particularly preferably represents hydrogen,
   represents C₁-C₁₀-alkyl, C₂-C₁₀-alkenyl or C₂-C₁₀-alkynyl, each of which is optionally mono- to tetrasubstituted by identical or different substituents from the group consisting of halogen, methoxy, ethoxy, methoxycarbonyl, ethoxycarbonyl, cyclopentyl, cyclohexyl, cycloheptyl (where cyclopentyl, cyclohexyl and cycloheptyl for their part may be substituted by methyl, ethyl, i-propyl), phenyl, phenyloxy, benzyloxy (which for their part may in each case be substituted by fluorine, chlorine, bromine, methyl, ethyl, n-, i-propyl, n-, s-, t-butyl, tri-fluoromethyl, trichloromethyl, phenyl or phenoxy, where the latter phenyl or phenoxy substituents for their part may be substituted by chlorine, cyano, or trifluoromethyl), thienyl (which for its part may be substituted by fluorine, chlorine, bromine, methyl or trifluoromethyl), isoxazolyl (which for its part may be substituted by fluorine, chlorine, bromine, methyl or trifluoromethyl),
   represents cyclohexyl, cyclohexenyl, phenyl, thienyl, isoxazolyl or pyridinyl, each of which may optionally be mono- to tetrasubstituted by identical or different substituents from the group consisting of fluorine, chlorine, bromine, methyl, ethyl, n-, i-propyl, n-, s-, t-butyl, trifluoromethyl, phenoxy, chlorophenoxy, dichlorophenoxy, chlorotrifluoromethylphenoxy.
R⁹ and R¹⁰ furthermore together with the carbon atom to which they are attached preferably form a C₃-C₇-cycloalkyl or C₃-C₇-cycloalkenyl ring which may be fused to a phenyl ring and which may furthermore optionally be substituted by C₁-C₄-alkyl or C₁-C₄-alkoxy.
R⁹ and R¹⁰ furthermore together with the carbon atom to which they are attached particularly preferably form a cyclopropyl, cyclopentyl, cyclopentenyl, cyclohexyl, cyclohexenyl, cycloheptyl ring which may be fused to a phenyl ring and which may furthermore optionally be substituted by methyl, n-propyl or methoxy.
R¹¹ preferably represents hydrogen, C₁-C₄-alkyl, C₁-C₄-haloalkyl, C₂-C₄-alkenyl, C₂-C₄-alkynyl, C₃-C₆-cycloalkyl or (C₁-C₄-alkoxy)carbonyl.
R¹¹ particularly preferably represents hydrogen, methyl or ethyl.
M preferably represents cycloalkylene which is mono- or polysubstituted by R¹⁸ or cycloalkenylene which is mono- to tetrasubstituted by R¹⁹, represents phenylene, naphthylene, thiophenylene, pyridinylene, pyrimidinylene, pyridazinylene or pyrazinylene, each of which is mono- or polysubstituted by R²⁰, or represents thiazolylene which is monosubstituted by R²¹.
M preferably represents one of the cycles below

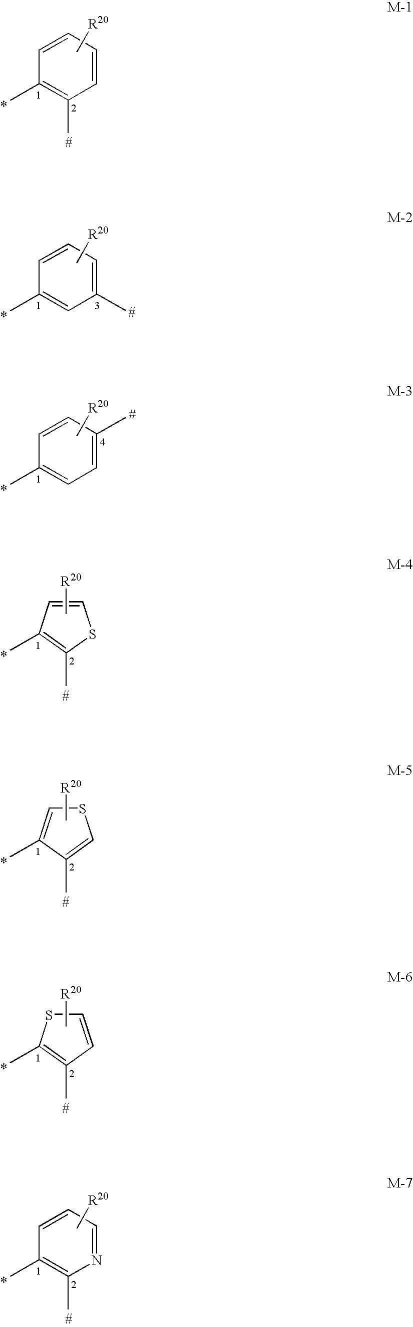

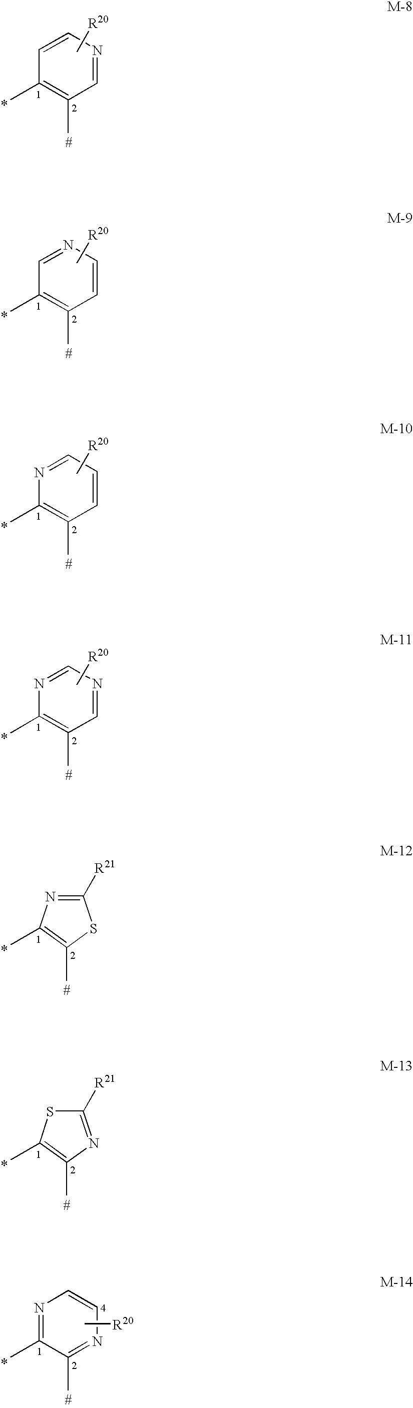

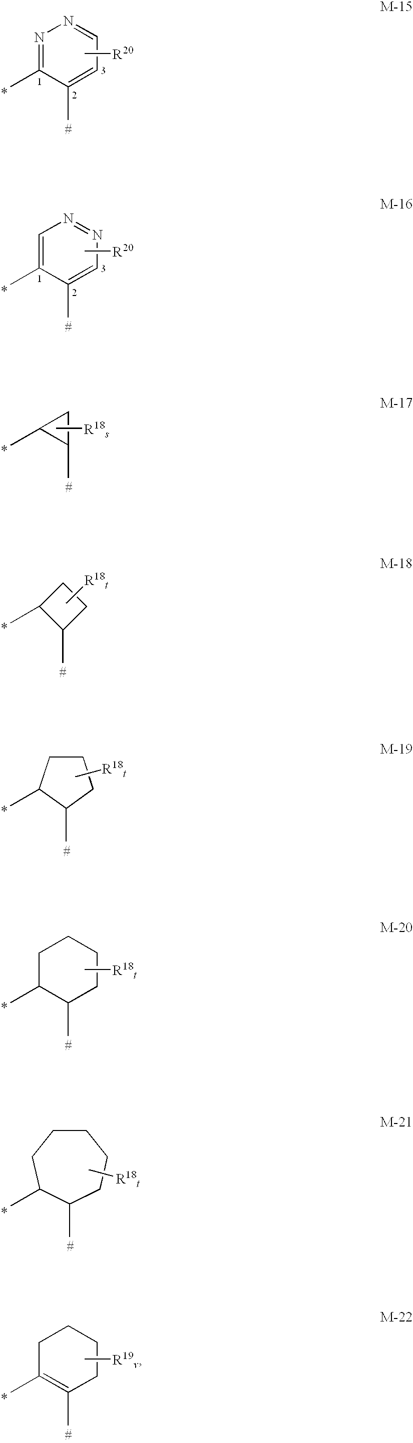

where the bond marked “#” is attached to the radical Q-Z.
M particularly preferably represents a cycle selected from the group consisting of M-1, M-2, M-3, M-4, M-5, M-6, M-7, M-8, M-11, M-12, M-13, M-17, M-19, M-20 and M-22.
M very particularly preferably represents a cycle selected from the group consisting of M-1, M-2, M-3, M-4, M-7, M-8, M-11, M-12, M-13, M-20 and M-22.
M especially preferably represents M-1 or M-3.
R¹⁸ represents hydrogen, fluorine, chlorine or C₁-C₄-alkyl, where the radicals R¹⁸ are identical or different if s or t is greater than 1.
R¹⁸ preferably represents hydrogen, fluorine, chlorine, methyl, ethyl, n- or i-propyl, n-, s- or t-butyl, where the radicals R¹⁸ are identical or different if s or t are greater than 1.
R¹⁸ particularly preferably represents hydrogen or methyl, where the radicals R¹⁸ are identical or different if s or t are greater than 1.
s represents 1 or 2.
t represents 1, 2, 3 or 4.
R¹⁹ represents hydrogen, fluorine, chlorine, methyl, ethyl, n-, i-propyl, n-, s- or t-butyl.
R¹⁹ particularly preferably represents hydrogen, fluorine, chlorine or methyl.
R¹⁹ very particularly preferably represents hydrogen or methyl.
v represents 1, 2, 3 or 4.
v preferably represents 1 or 2.
v particularly preferably represents 1.
R²⁰ represents hydrogen, cyano, fluorine, chlorine, bromine, C₁-C₄-alkyl, C₁-C₄-alkoxy, C₁-C₄-alkylthio, C₁-C₄-haloalkyl, C₁-C₄-haloalkoxy.
R²⁰ preferably represents hydrogen, fluorine, chlorine, methyl, methoxy, methylthio, trifluoromethyl or trifluoromethoxy.
R²⁰, if M represents M-1, M-2 or M-3, particularly preferably represents hydrogen, fluorine, chlorine, methyl or trifluoromethyl.
R²⁰, if M represents M-4, M-5 or M-6, furthermore particularly preferably represents hydrogen, chlorine or methyl.
R²⁰, if M represents M-7, M-8, M-9 or M-10, furthermore, particularly preferably represents hydrogen, fluorine, chlorine or methyl.
R²⁰, if M represents M-11, M-14, M-15 or M-16, furthermore particularly preferably represents hydrogen, methyl or trifluoromethyl.
R²⁰ very particular preferably represents hydrogen.
R²¹ represents hydrogen, methyl, methylthio or trifluoromethyl.
R²¹ preferably represents hydrogen.
R²¹ furthermore preferably represents methyl.
R²¹ furthermore preferably represents trifluoromethyl.
Q preferably represents a direct bond.
Q furthermore preferably represents —CH₂—, —(CH₂)₂—, —(CH₂)₃—, —CH(CH₃)—, —C(CH₃)₂—, —CH₂O—, particularly preferably represents —CH₂—, —(CH₂)₂—, —OCH₂—, —CH₂O—.
Q furthermore preferably represents —CH═CH—, —CH₂—CH═CH—, —CH(CH₃)—CH═CH—, particularly preferably represents —CH═CH—.
Q furthermore preferably represents O, S, SO, SO₂ particularly preferably represents O.
Q furthermore preferably represents NR¹², particularly preferably represents NH.
Q very particularly preferably represents a direct bond.
Z preferably represents hydrogen, where Q does not represent O, S, SO, SO₂, NR¹².
Z preferably represents Z¹.
Z¹ preferably represents phenyl which is optionally mono- to pentasubstituted by identical or different substituents, where the substituents are in each case selected from the list W¹.
Z¹ particularly preferably represents unsubstituted phenyl.
Z¹ also particularly preferably represents monosubstituted phenyl, where the substituents are selected from the list W¹.
Z¹ also particularly preferably represents phenyl which is disubstituted by identical or different substituents, where the substituents are selected from the list W¹.
Z¹ also particularly preferably represents phenyl which is trisubstituted by identical or different substituents, where the substituents are selected from the list W¹.
Z¹ very particularly preferably represents phenyl which is monosubstituted in the 4-position, where the substituents are selected from the list W¹.
Z¹ very particularly preferably represents phenyl which is disubstituted by identical or different substituents in the 3,4-position, where the substituents are selected from the list W¹.
Z¹ very particularly preferably represents phenyl which is disubstituted by identical or different substituents in the 2,3-position, where the substituents are selected from the list W¹.
Z¹ very particularly preferably represents phenyl which is disubstituted by identical or different substituents in the 2,4-position, where the substituents are selected from the list W¹.
Z¹ very particularly preferably represents phenyl which is disubstituted by identical or different substituents in the 3,5-position, where the substituents are selected from the list W¹.
Z¹ very particularly preferably represents phenyl which is trisubstituted by identical or different substituents in the 2,4,6-position, where the substituents are selected from the list W¹.
W¹ represents halogen, cyano, nitro, formyl, carboxy, carbamoyl, thiocarbamoyl;
   in each case straight-chain or branched alkyl, hydroxyalkyl, oxoalkyl, alkoxy, alkoxyalkyl, alkylthioalkyl, dialkoxyalkyl, alkylthio, alkylsulphynyl or alkylsulphonyl having in each case 1 to 8 carbon atoms;
   in each case straight-chain or branched alkenyl or alkenyloxy having in each case 2 to 6 carbon atoms;
   in each case straight-chain or branched haloalkyl, haloalkoxy, haloalkylthio, haloalkylsulphynyl or haloalkylsulphonyl having in each case 1 to 6 carbon atoms and 1 to 13 identical or different halogen atoms;
   in each case straight-chain or branched haloalkenyl or haloalkenyloxy having in each case 2 to 6 carbon atoms and 1 to 11 identical or different halogen atoms;
   in each case straight-chain or branched alkylamino, dialkylamino, alkylcarbonyl, alkylcarbonyloxy, alkoxycarbonyl, alkylaminocarbonyl, dialkylaminocarbonyl, arylalkylaminocarbonyl, dialkylaminocarbonyloxy having 1 to 6 carbon atoms in the respective hydrocarbon chains, alkenylcarbonyl or alkynylcarbonyl having 2 to 6 carbon atoms in the respective hydrocarbon chains;
   cycloalkyl or cycloalkyloxy having in each case 3 to 6 carbon atoms;
   in each case doubly attached alkylene having 3 or 4 carbon atoms, oxyalkylene having 2 or 3 carbon atoms or dioxyalkylene having 1 or 2 carbon atoms, each of which is optionally mono- to tetrasubstituted by identical or different substituents from the group consisting of fluorine, chlorine, oxo, methyl, trifluoromethyl and ethyl;
   or the groupings —(CR¹³R¹⁴)ₘSiR¹⁵R¹⁶R¹⁷ or —C(Q¹)═N-Q², in which
   Q¹ represents hydrogen, hydroxy or alkyl having 1 to 4 carbon atoms, haloalkyl having 1 Q¹ to 4 carbon atoms and 1 to 9 fluorine, chlorine and/or bromine atoms or cycloalkyl having 1 to 6 carbon atoms and
   Q² represents hydroxy, amino, methylamino, phenyl, benzyl or represents in each case optionally cyano-, hydroxy-, alkoxy-, alkylthio-, alkylamino-, dialkylamino- or phenyl-substituted alkyl or alkoxy having 1 to 4 carbon atoms, or represents alkenyloxy or alkynyloxy having in each case 2 to 4 carbon atoms,
   and also phenyl, phenoxy, phenylthio, benzoyl, benzoylethenyl, cinnamoyl, heterocyclyl or phenylalkyl, phenylalkyloxy, phenylalkylthio, or heterocyclylalkyl having in each case 1 to 3 carbon atoms in the respective alkyl moieties, each of which radicals is optionally mono- to trisubstituted in the cyclic moiety by halogen and/or straight-chain or branched alkyl or alkoxy having 1 to 4 carbon atoms.
W¹ preferably represents fluorine, chlorine, bromine, cyano, methyl, ethyl, n- or i-propyl, n-, s- or t-butyl, methoxy, ethoxy, n- or i-propoxy, trifluoromethyl, trifluoroethyl, difluoromethoxy, trifluoromethoxy, difluorochloromethoxy, trifluoroethoxy, in each case doubly attached difluoromethylenedioxy or tetrafluoroethylenedioxy,
   or the groupings —CH₂Si(CH₃)₃, —Si(CH₃)₃ or —C(Q¹)═N-Q² in which
   Q¹ represents hydrogen, methyl, ethyl or trifluoromethyl and
   Q² represents hydroxy, methoxy, ethoxy, propoxy or isopropoxy.
Z preferably represents Z².
Z² preferably represents 2-pyridinyl, 3-pyridinyl or 4-pyridinyl, optionally mono- to trisubstituted by identical or different substituents, where the substituents are in each case selected from the list W².
Z² particularly preferably represents monosubstituted 2-pyridinyl, 3-pyridinyl or 4-pyridinyl, where the substituents are in each case selected from the list W².
Z² also particularly preferably represents 2-pyridinyl, 3-pyridinyl or 4-pyridinyl which is disubstituted by identical or different substituents, where the substituents are in each case selected from the list W².
Z² also particularly preferably represents 2-pyridinyl, 3-pyridinyl or 4-pyridinyl which is trisubstituted by identical or different substituents, where the substituents are in each case selected from the list W².
Z² very particularly preferably represents 2-pyridinyl which is monosubstituted in the 5-position or 3-pyridinyl which is monosubstituted in the 6-position, where the substituents are in each case selected from the list W².
Z² very particularly preferably represents 2-pyridinyl which is disubstituted by identical or different substituents in the 3,5-position, where the substituents are selected from the list W².
Z² very particularly preferably represents 3-pyridinyl which is disubstituted by identical or different substituents in the 4,6-position, where the substituents are selected from the list W².
Z² very particularly preferably represents 4-pyridinyl which is disubstituted by identical or different substituents in the 3,5-position, where the substituents are selected from the list W².
W² represents hydrogen, halogen, cyano, nitro, C₁-C₆-alkyl, C₂-C₄-alkenyl, C₂-C₄-alkynyl, C₁-C₄-alkoxy, C₁-C₄-alkylthio, C₁-C₄-alkylsulphynyl, C₁-C₄-alkylsulphonyl, C₃-C₆-cycloalkyl; represents C₁-C₄-haloalkyl, C₁-C₄-haloalkoxy, C₁-C₄-haloalkylthio, C₁-C₄-haloalkylsulphynyl, C₁-C₄-haloalkylsulphonyl having in each case 1 to 5 halogen atoms; represents —SO₂NR²²R²³, —C(═O)R²⁴, —C(═S)R²⁴, —Si(R²⁵)₃, C₂-C₄-alkenylene-Si(R²⁵)₃, C₂-C₄-alkynylene-Si(R²⁵)₃, —NR²²R²³, —CH₂—NR²²R²³ in which
   R²² represents hydrogen, C₁-C₄-alkyl, —C(═O)R²⁴ or —C(═S)R²⁴,
   R²³ represents hydrogen, C₁-C₄-alkyl, —C(═O)R²⁴ or —C(═S)R²⁴,
   R²² and R²³ furthermore together with the nitrogen atom to which they are attached form a saturated heterocycle having 5 to 8 ring atoms which is optionally mono- or polysubstituted by identical or different substituents from the group consisting of halogen and C₁-C₄-alkyl, where the heterocycle may contain 1 or 2 further non-adjacent heteroatoms from the group consisting of oxygen, sulphur and NR²⁶,
   R²⁴ represents hydrogen, C₁-C₄-alkyl, C₁-C₄-alkoxy or —NR²⁷R²⁸,
   R²⁵ represents hydrogen, C₁-C₈-alkyl, C₁-C₈-alkoxy, C₁-C₄-alkoxy-C₁-C₄-alkyl, C₁-C₄-alkylthio-C₁-C₄-alkyl or C₁-C₆-haloalkyl, where the three radicals R²⁵ may in each case be identical or different,
   R²⁶ represents hydrogen or C₁-C₆-alkyl,
   R²⁷ represents hydrogen or C₁-C₄-alkyl,
   R²⁸ represents hydrogen or C₁-C₄-alkyl,
   R²⁷ and R²⁸ furthermore together with the nitrogen atom to which they are attached form a saturated heterocycle having 5 to 8 ring atoms which is optionally mono- or polysubstituted by identical or different substituents from the group consisting of halogen and C₁-C₄-alkyl, where the heterocycle may contain 1 or 2 further non-adjacent heteroatoms from the group consisting of oxygen, sulphur and NR²⁶,
W² preferably represents hydrogen, fluorine, chlorine, bromine, cyano, nitro, methyl, ethyl, n- or isopropyl, n-, iso-, sec- or tert-butyl, allyl, propargyl, methoxy, ethoxy, n- or isopropoxy, n-, iso-, sec- or tert-butoxy, methylthio, ethylthio, n- or isopropylthio, n-, iso-, sec- or tert-butylthio, methylsulphynyl, ethylsulphynyl, n- or isopropylsulphynyl, n-, iso-, sec- or tert-butylsulphynyl, methylsulphonyl, ethylsulphonyl, n- or isopropylsulphonyl, n-, iso-, sec- or tert-butylsulphonyl, cyclopropyl, cyclopentyl, cyclohexyl, trifluoromethyl, difluoromethyl, trichloromethyl, trifluoroethyl, trifluoromethoxy, difluoromethoxy, trichloromethoxy, difluoromethylthio, difluorochloromethylthio, trifluoromethylthio, trifluoromethylsulphynyl, trifluoromethylsulphonyl, —SO₂NMe₂, —C(═O)R²⁴, —C(═S)R²⁴, —Si(R²⁵)₃, —CH═CH—Si(R²⁵)₃, —CH₂—CH═CH—Si(R²⁵)₃, —CH═CH—CH₂—Si(R²⁵)₃, —C≡C—Si(R²⁵)₃, —CH₂—C≡C—Si(R²⁵)₃, —C≡C—CH₂—Si(R²⁵)₃, —CH₂—C≡C—CH₂—si(R²⁵)₃, —NR²²R²³, —CH₂—NR²²R²³.
R²² preferably represents hydrogen, methyl, ethyl, n- or isopropyl, —C(═O)R²⁴ or —C(═S)R²⁴.
R²² particularly preferably represents hydrogen or methyl.
R²³ preferably represents hydrogen, methyl, ethyl, n- or isopropyl, —C(═O)R²⁴ or —C(═S)R²⁴.
R²³ particularly preferably represents hydrogen or methyl.
R²² and R²³ furthermore together with the nitrogen atom to which they are attached preferably form a saturated heterocycle from the group consisting of morpholine, thiomorpholine and piperazine, which is optionally mono- to tetrasubstituted by identical or different substituents from the group consisting of fluorine, chlorine, bromine and methyl, where the piperazine may be substituted at the second nitrogen atom by R²⁶.
R²⁴ preferably represents hydrogen, methyl, ethyl, n- or isopropyl, methoxy, ethoxy, n- or isopropoxy or —NR²⁷R²⁸.
R²⁴ particularly preferably represents hydrogen, methyl, ethyl, methoxy, ethoxy or —NR²⁷R²⁸.
R²⁵ preferably represents methyl, ethyl, methoxy, ethoxy, methoxymethyl, ethoxymethyl, methoxyethyl, ethoxyethyl, methylthiomethyl, ethylthiomethyl, methylthioethyl or ethylthioethyl, where the three radicals R²⁵ may in each case be identical or different.
R²⁵ particularly preferably represents methyl, methoxy, methoxymethyl or methylthiomethyl, where the three radicals R²⁵ may in each case be identical or different.
R²⁵ very particularly preferably represents methyl.
R²⁶ preferably represents hydrogen or C₁-C₄-alkyl.
R²⁶ particularly preferably represents hydrogen, methyl, ethyl, n- or isopropyl, n-, iso-, sec- or tert-butyl.
R²⁷ preferably represents hydrogen, methyl, ethyl, n- or isopropyl.
R²⁷ particularly preferably represents hydrogen or methyl.
R²⁸ preferably represents hydrogen, methyl, ethyl, n- or isopropyl.
R²⁸ particularly preferably represents hydrogen or methyl.
R²⁷ and R²⁸ furthermore together with the nitrogen atom to which they are attached preferably form a saturated heterocycle from the group consisting of morpholine, thiomorpholine and piperazine, which is optionally mono- to tetrasubstituted by identical or different substituents from the group consisting of fluorine, chlorine, bromine and methyl, where the piperazine may be substituted at the second nitrogen atom by R²⁶.
Z also preferably represents Z³.
Z³ preferably represents cycloalkyl or bicycloalkyl having in each case 3 to 10 carbon atoms, each of which radicals is optionally mono- to tetrasubstituted by identical or different substituents from the group consisting of halogen, C₁-C₄-alkyl, C₃-C₆-cycloalkyl, —CH₂Si(CH₃)₃ and/or —Si(CH₃)₃.
Z³ particularly preferably represents cyclopropyl, cyclopentyl, cyclohexyl, cycloheptyl, cyclooctyl, cyclononyl, bicyclo[2.2.1]heptyl or bicyclo[2.2.2]octyl, each of which is optionally mono- to tetrasubstituted by identical or different substituents from the group consisting of chlorine, methyl, cyclopropyl, —CH₂Si(CH₃)₃ and/or —Si(CH₃)₃.
Z³ very particularly preferably represents cyclopropyl which is optionally substituted by chlorine and/or methyl.
Z also preferably represents Z⁴.
Z⁴ preferably represents unsubstituted C₁-C₂₀-alkyl or represents C₁-C₂₀-alkyl, which is mono- or polysubstituted by identical or different substituents from the group consisting of fluorine, chlorine, bromine, iodine, C₁-C₆-alkylthio, C₁-C₆-alkylsulphynyl, C₁-C₆-alkylsulphonyl, C₁-C₆-alkoxy, C₁-C₆-alkylamino, di(C₁-C₆-alkyl)amino, C₁-C₆-haloalkylthio, C₁-C₆-haloalkylsulphynyl, C₁-C₆-haloalkylsulphonyl, C₁-C₆-haloalkoxy, C₁-C₆-haloalkylamino, halo-di(C₁-C₆-alkyl)amino, —SiR¹⁵R¹⁶R¹⁷ and C₃-C₆-cycloalkyl, where the cycloalkyl moiety for its part may optionally be mono- to tetrasubstituted by identical or different substituents from the group consisting of fluorine, chlorine, bromine, iodine, C₁-C₄-alkyl and C₁-C₄-haloalkyl.
Z⁴ Particularly preferably represents unsubstituted C₁-C₂₀-alkyl.
Z⁴ also particularly preferably represents C₁-C₂₀-alkyl which is substituted by fluorine, chlorine, bromine, iodine, C₁-C₆-alkylthio, C₁-C₄-alkylsulphynyl, C₁-C₄-alkylsulphonyl, C₁-C₄-alkoxy, C₁-C₄-alkylamino, di(C₁-C₄-alkyl)amino, C₁-C₄-haloalkylthio, C₁-C₄-haloalkylsulphynyl, C₁-C₄-haloalkylsulphonyl, C₁-C₄-haloalkoxy, C₁-C₄-haloalkylamino, halo-di(C₁-C₄-alkyl)amino having in each case 1 to 9 fluorine, chlorine and/or bromine atoms, —SiR¹⁵R¹⁶R¹⁷, cyclopropyl, dichlorocyclopropyl, cyclobutyl, cyclopentyl or cyclohexyl; vow particularly preferably represents C₁-C₂₀-alkyl which is substituted by fluorine, chlorine, methylthio, ethylthio, n- or isopropylthio, n-, iso-, sec-, tert-butylthio, pentylthio, hexylthio, methylsulphonyl, ethylsulphonyl, n- or isopropylsulphonyl, n-, iso-, sec-, tert-butylsulphonyl, methoxy, ethoxy, n- or isopropoxy, n-, iso-, sec-, tert-butoxy, methylamino, ethylamino, n- or isopropylamino, n-, iso-, sec-, tert-butylamino, dimethylamino, diisopropylamino, trifluoromethylthio, trifluoromethoxy, —SiR¹⁵R¹⁶R¹⁷, cyclopropyl, dichlorocyclopropyl, cyclobutyl, cyclopentyl or cyclohexyl.
Z also preferably represents Z⁵.
Z⁵ preferably represents C₂-C₂₀-alkenyl or C₂-C₂₀-alkynyl, each of which is optionally mono- or polysubstituted by identical or different substituents from the group consisting of fluorine, chlorine, bromine, iodine, C₁-C₆-alkylthio, C₁-C₆-alkylsulphynyl, C₁-C₆-alkylsulphonyl, C₁-C₆-alkoxy, C₁-C₆-alkylamino, di(C₁-C₆-alkyl)amino, C₁-C₆-haloalkylthio, C₁-C₆-haloalkylsulphynyl, C₁-C₆-haloalkylsulphonyl, C₁-C₆-haloalkoxy, C₁-C₆-haloalkylamino, halo-di(C₁-C₆-alkyl)amino, —SiR¹⁵R¹⁶R¹⁷ and C₃-C₆-cycloalkyl, where the cycloalkyl moiety for its part may optionally be mono- to tetrasubstituted by identical or different substituents from the group consisting of fluorine, chlorine, bromine, iodine, C₁-C₄-alkyl and C₁-C₄-haloalkyl.
Z⁵ Particularly preferably represents C₂-C₂₀-alkenyl or C₂-C₂₀-alkynyl, each of which is optionally substituted by fluorine, chlorine, bromine, iodine, C₁-C₆-alkylthio, C₁-C₄-alkylsulphynyl, C₁-C₄-alkylsulphonyl, C₁-C₄-alkoxy, C₁-C₄-alkylamino, di(C₁-C₄-alkyl)amino, C₁-C₄-haloalkylthio, C₁-C₄-haloalkylsulphynyl, C₁-C₄-haloalkylsulphonyl, C₁-C₄-haloalkoxy, C₁-C₄-haloalkylamino, halodi(C₁-C₄-alkyl)amino having in each case 1 to 9 fluorine, chlorine and/or bromine atoms, —SiR¹⁵R¹⁶R¹⁷ cyclopropyl, dichlorocyclopropyl, cyclobutyl, cyclopentyl or cyclohexyl.
Z⁵ very particularly preferably represents C₂-C₂₀-alkenyl or C₂-C₂₀-alkynyl.
Z also preferably represents Z⁶.
Z⁶ preferably represents one of the rings below

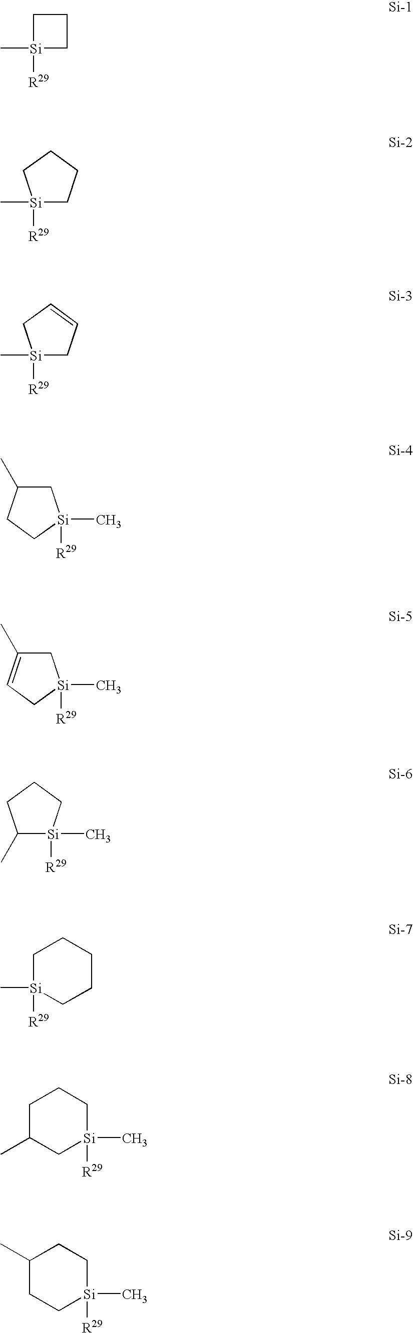

in which R²⁹ represents hydrogen or methyl.
M-Q-Z also preferably together represent 1,1,3-trimethyl-1H-2,3-dihydroinden-4-yl, 1,3-dimethyl-1H-2,3-dihydroinden-4-yl, 1,1,3-trimethyl-1,3-dihydro-2-benzofuran-4-yl, 1,3-dimethyl-1,3-dihydro-2-benzofuran-4-yl, 1,1,3-trimethyl-1,3-dihydro-2-benzothien-4-yl, 1,3-dimethyl-1,3-dihydro-2-benzothien-4-yl or 1,2,3,4-tetrahydro-9-isopropyl-1,4-methanonaphthalen-5-yl.
M-Q-Z also particularly preferably together represent 1,1,3-trimethyl-1H-2,3-dihydroinden-4-yl or 1,2,3,4-tetrahydro-9-isopropyl-1,4-methanonaphthalen-5-yl.
R¹² preferably represents hydrogen, C₁-C₆-alkyl, C₁-C₃-alkoxy-C₁-C₃-alkyl, C₁-C₃-alkylthio-C₁-C₃-alkyl or C₃-C₆-cycloalkyl.
R¹² particularly preferably represents hydrogen, methyl, ethyl, n- or isopropyl, n-, sec-, iso- or tert-butyl, methoxymethyl, ethoxymethyl, methoxyethyl, ethoxyethyl, methylthiomethyl, ethylthiomethyl, methylthioethyl, ethylthioethyl or cyclopropyl.
R¹² very particularly preferably represents hydrogen, methyl, ethyl, methoxymethyl or methylthiomethyl.
R¹³ preferably represents hydrogen or methyl.
R¹³ particularly preferably represents hydrogen.
R¹⁴ preferably represents hydrogen or methyl.
R¹⁴ particularly preferably represents hydrogen.
m preferably represents 0, 1 or 2.
R¹⁵ and R¹⁶ independently of one another preferably represent C₁-C₆-alkyl, C₁-C₆-alkoxy, C₁-C₃-alkoxy-C₁-C₃-alkyl or C₁-C₃-alkylthio-C₁-C₃-alkyl.
R¹⁵ and R¹⁶ independently of one another particularly preferably represent methyl, ethyl, methoxy, ethoxy, methoxymethyl, ethoxymethyl, methoxyethyl, ethoxyethyl, methylthiomethyl, ethylthiomethyl, methylthioethyl or ethylthioethyl.
R¹⁵ and R¹⁶ independently of one another very particularly preferably represent methyl, methoxy, methoxymethyl or methylthiomethyl.
R¹⁵ and R¹⁶ especially preferably each represent methyl.
R¹⁷ preferably represents C₁-C₆-alkyl, C₁-C₆-alkoxy, C₁-C₃-alkoxy-C₁-C₃-alkyl, C₁-C₃-alkylthio-C₁-C₃-alkyl, C₃-C₆-cycloalkyl, phenyl or benzyl.
R¹⁷ particularly preferably represents methyl, ethyl, n- or isopropyl, n-, sec-, iso- or tert-butyl, methoxy, ethoxy, n- or isopropoxy, n-, sec-, iso- or tert-butoxy, methoxymethyl, ethoxymethyl, methoxyethyl, ethoxyethyl, methylthiomethyl, ethylthiomethyl, methylthioethyl, ethylthioethyl, cyclopropyl, phenyl or benzyl.
R¹⁷ particularly preferably represents methyl, ethyl, n- or isopropyl, iso- or tert-butyl, methoxy, isopropoxy, iso- or tert-butoxy, methoxymethyl, methylthiomethyl or phenyl.
R¹⁷ especially preferably represents methyl, ethyl, n- or isopropyl, iso- or tert-butyl, methoxy, isopropoxy, iso- or tert-butoxy.
R¹⁷ most preferably represents methyl.

The compounds of the formulae (I) and (II) are in particular tautomeric forms when R¹ represents hydrogen and R⁴ represents hydrogen:

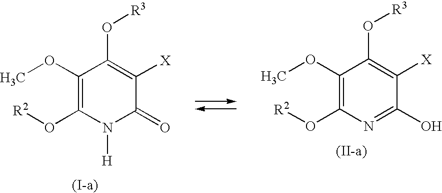

It has now been found, that 2-pyridones of the formula (I)

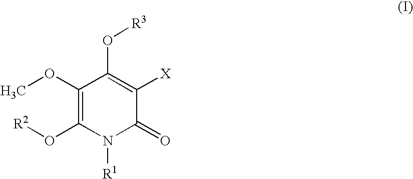

in which X, R¹, R² and R³ have the meanings given above

are obtained when

according to process (1)
(a) oxymalonic acid derivatives of the formula (III)

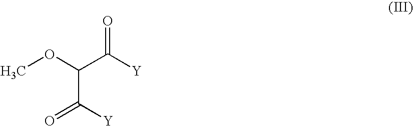

in which
Y represents halogen, hydroxyl or —O-AM in which
AM represents an alkali metal selected from the group consisting of lithium, sodium, potassium and caesium,
are reacted with amides of the formula (IV)

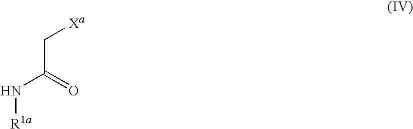

in which
R^{1a} represents alkyl, alkenyl, alkynyl, cycloalkyl, aryl, hetaryl, optionally mono- or polysubstituted by identical or different substituents,
X^{a} represents —C(═O)OR^{5a}, —C(═O)CR⁸R⁹R¹⁰ or represents —C(═O)aryl or —(C═O)hetaryl which is optionally substituted in the aryl or heteraryl moiety, and
R^{5a} represents alkyl, alkenyl, alkynyl, cycloalkyl, cycloalkenyl, bicycloalkyl, aryl, hetaryl, optionally mono- or polysubstituted by identical or different substituents,
   and
R⁸, R⁹ and R¹⁰ have the meanings given above,
if appropriate in the presence of a diluent,
thus giving 2-pyridones of the formula (I-b)

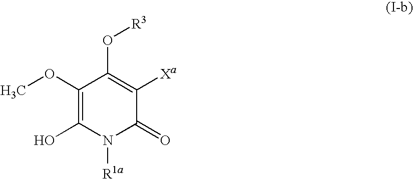

in which X^{a}, R^{1a} and R³ have the meanings given above

which are subsequently
(b) if appropriate reacted with an alkylating agent, if appropriate in the presence of a base, a Lewis acid or a molecular sieve and, if appropriate, in the presence of a diluent,
   giving 2-pyridones of the formula (I-c)

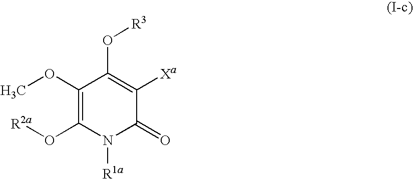

in which
X^{a}, R^{1a} and R³ have the meanings given above, and
R^{2a} represents methyl or ethyl,

after which
(c) if appropriate the 2-pyridones of the formula (I-b) or the formula (I-c) are hydrogenated in the presence of a transition metal catalyst or in the presence of Lewis or Brønsted acids, thus giving 2-pyridones of the formula (I-d)

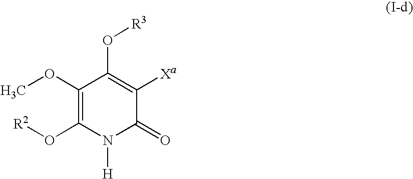

in which R², R³ and X^{a} have the meanings given above,

and
(d) if appropriate, the 2-pyridones of the formula (I-b) or the formula (I-c) or the formula (I-d), in which X^{a} represents —C(═O)OR^{5a} are cleaved, thus giving the 2-pyridones of the formula (I-e)

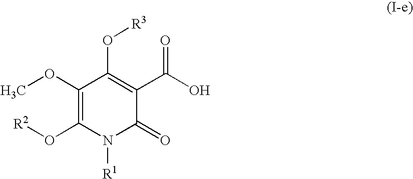

in which R¹, R² and R³ have the meanings given above;

or according to process (2)
(e) 2-pyridones of the formula (I-f)

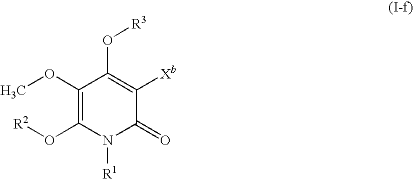

in which
R¹, R² and R³ have the meanings given above and
X^{b} represents —C(═O)OH or —C(═O)OR^{5b} in which
R^{5b} represents C₁-C₄-alkyl,
are reacted with amines of the formula (VIII)

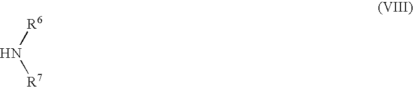

in which R⁶ and R⁷ have the meanings given above,
if appropriate in the presence of diluents and if appropriate in the presence of reaction auxiliaries, thus giving the 2-pyridones of the formula (I-g)

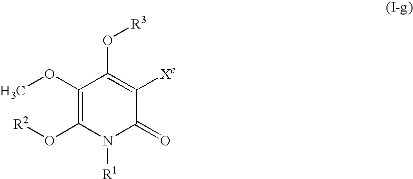

in which
R¹, R² and R³ have the meanings given above and
X^{c} represents —C(═O)NR⁶R⁷;

or according to process (3)
(f) 2-pyridones of the formula (I-e)

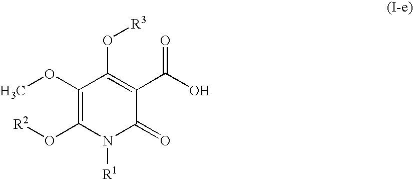

in which R¹, R² and R³ have the meanings given above
are reacted, if appropriate in the presence of diluents and if appropriate in the presence of basic reaction auxiliaries, thus giving the 2-pyridones of the formula (I-h)

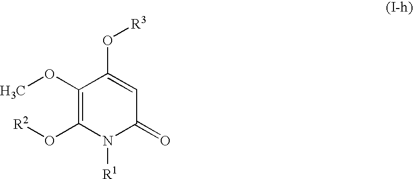

in which R¹, R² and R³ have the meanings given above;

and that 2-pyridinols of the formula (II)

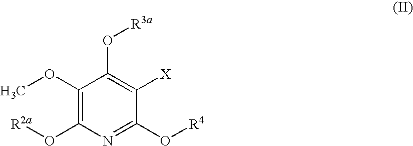

in which

X and R^{2a} have the meanings given above, and

R^{3a} and R⁴ each represent acetyl

are obtained by reacting,

according to process (4),
(g) 2-pyridones of the formula (I-i)

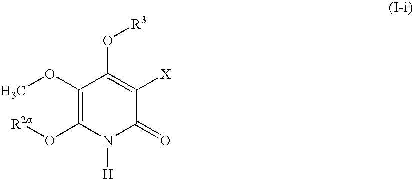

in which R^{2a}, R³ and X have the meanings given above
with an acetylating agent, if appropriate in the presence of a diluent and if appropriate in the presence of a basic reaction auxiliary.

The 2-pyridones of the general formula (I-k)

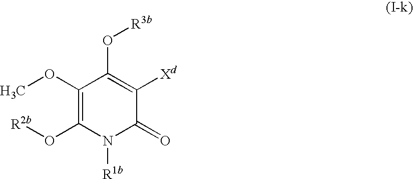

and the 2-pyridinols of the general formula (II-b)

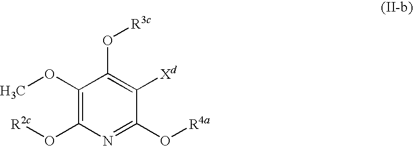

which can be prepared by the process according to the invention and in which

X^{d} has the meanings of X,

R^{1b} has the meanings of R¹,

R^{2b} has the meanings of R²,

R^{2c} has the meanings of R^{2a},

R^{3b} has the meanings of R³,

R^{3c} has the meanings of R^{3a},

R^{4a} has the meanings of R⁴,

are novel and also form part of the subject-matter of the present application,

except for compounds in which
(1) X^{d} represents —C(═O)CR^{8a}R^{9a}R^{10b},
   R^{1b}, R^{3b}, R^{3c}, R^{4a} and R^{8a} each represent hydrogen,
   R^{2b}, R^{2c} and R^{9a} each represent methyl,
   R^{10b} represents alkyl or alkenyl, each of which is optionally mono- or polysubstituted by identical or different halogen or represents phenyl or represents 4-tert-butylbenzyl;
(2) X^{d} represents —C(═O)CR^{8b}R^{9b}R^{10c},
   R^{1b}, R^{3b} and R^{8b} each represent hydrogen,
   R^{2b}, R^{2c} and R^{9b} each represent methyl,
   R^{3c} and R^{4a} each represent acetyl,
   R^{10c} represents 2-butenyl or 2-methyl-3,3,4-trichlorobutyl;
(3) X^{d} represents —C(═O)CR^{8c}R^{9c}R^{10d},
   R^{1b}, R^{3b}, R^{3c}, R^{4a} and R^{8a} each represent hydrogen,
   R^{2b} and R^{2c} each represent methyl,
   R^{9c} represents ethyl,
   R^{10d} represents n-butyl;
(4) X^{d} represents —C(═O)CR^{8d}R^{9d}R^{10e},
   R^{1b}, R^{3b}, R^{3c}, R^{4a} and R^{8d} each represent hydrogen,
   R^{2b} and R^{2c} each represent methyl,
   R^{9d} and R^{10e} together with the carbon atom to which they are attached form an unsubstituted cyclohexyl ring;
(5) X^{d} represents unsubstituted —C(═O)phenyl, —C(═O)-4-chlorophenyl or —C(═O)-3-phenoxyphenyl,
   R^{1b}, R^{3b}, R^{3c} and R^{4a} each represent hydrogen and
   R^{2b} and R^{2c} each represent methyl;
(6) X^{d} represents —C(═O)OR^{5c} or —C(═O)NR^{6a}R^{7a},
   R^{1b}, R^{3b}, R^{3c} and R^{4a} each represent hydrogen,
   R^{2b} represents hydrogen, methyl or ethyl,
   R^{2c} represents methyl or ethyl,
   R^{5c} represents hydrogen or represents C₁-C₆-alkyl which is optionally mono- or polysubstituted by identical or different substituents,
   R^{6a} represents hydrogen, C₁-C₆-alkyl,
   R^{7a} represents phenyl-C₁-C₄-alkyl which may optionally be substituted in the phenyl ring, or represents -M^{a}-Q^{a}-Z^{a},
   M^{a} represents optionally substituted phenylene,
   Q^{a} represents a direct bond, C₁-C₄-alkylene, C₁-C₄-alkyleneoxy, oxy-C₁-C₄-alkylene, O, S or NR^{12a},
   R^{12a} represents hydrogen or C₁-C₆-alkyl,
   Z^{a} represents phenyl or pyridyl, optionally mono- to pentasubstituted by identical or different substituents, where the substituents are selected from the list W^{1a},
   W^{1a} represents halogen, cyano, nitro, formyl;
      in each case straight-chain or branched alkyl, alkoxy, alkylthio, alkylsulphynyl or alkylsulphonyl having in each case 1 to 6 carbon atoms;
      in each case straight-chain or branched haloalkyl, haloalkoxy, haloalkylthio, haloalkylsulphynyl or haloalkylsulphonyl having in each case 1 to 6 carbon atoms and 1 to 13 identical or different halogen atoms;
      in each case straight-chain or branched alkylamino, dialkylamino, alkylcarbonyloxy, alkoxycarbonyl having 1 to 6 carbon atoms in the respective hydrocarbon chains;
      in each case doubly attached dioxyalkylene, each of which is optionally mono- to tetrasubstituted by identical or different substituents from the group consisting of fluorine and chlorine and has 1 or 2 carbon atoms.

Finally, it has been found that the novel 2-pyridones of the formula (I-k) and the novel 2-pyridinols of the formula (II-b) have very good microbicidal properties and can be used for controlling unwanted microorganisms including fungi, both in crop protection and in the protection of materials.

If appropriate, the compounds according to the invention can be present as mixtures of various possible isomeric forms, in particular of stereoisomers, such as, for example, E and Z, threo and erythro, and also optical isomers, and, if appropriate, also of tautomers. What is claimed are both the E and the Z isomers, as well as the threo and erythro, and also the optical isomers, any mixtures of these isomers, and also the possible tautomeric forms.

According to the invention, the formula (I) provides a general definition of the 2-pyridones. According to the invention, the formula (II) provides a general definition of the 2-pyridinols. Preferred radical definitions of the formulae mentioned above and below have already been given above. These definitions apply to the end products and likewise to all intermediates.

According to the invention, the formula (I-k) provides a general definition of the 2-pyridones. According to the invention, the formula (II-b) provides a general definition of the 2-pyridinols.

In the formulae (I-k) and (II-b) X^{d} has in each case the preferred, particularly preferred and very particularly preferred meanings of X, R^{1b} has in each case the preferred, particularly preferred and very particularly preferred meanings of R¹, R^{2b} has in each case the preferred and particularly preferred meanings of R², R^{2c} has in each case the preferred and particularly preferred meanings of R^{2a}, R^{3b} has the meaning of R³, R^{3c} has the preferred meanings of R^{3a}, R^{4a} has the preferred meanings of R⁴,

with the exception of compounds in which
(1) X^{d} represents —C(═O)CR^{8a}R^{9a}R^{10b},
   R^{1b}, R^{3b}, R^{3c}, R^{4a} and R^{8a} each represent hydrogen,
   R^{2b}, R^{2c} and R^{9a} each represent methyl,
   R^{10b} represents methyl, ethyl, n- or isopropyl, n- or isobutyl, n- or isopentyl, 2-methyl-butyl, n-hexyl, 2-methylpentyl, 3-methylpentyl, 4-methylpentyl, 2-ethylbutyl, heptyl, octyl, 1-propenyl, allyl, 1-butenyl, 2-butenyl, 1-pentenyl, 2-pentenyl, butyl, heptyl, octyl, 1-propenyl, allyl, 1-butenyl, 2-butenyl, 1-pentenyl, 2-pentenyl, 3-pentenyl, 3-methyl-2-butenyl, geranyl, each of which is optionally mono- or polysubstituted by chlorine,
      represents in particular 2-methyl-3-chlorobutyl, 2-methyl-3,4-dichlorobutyl, 2-methylbutyl, n-butyl, 2-butenyl, 2-methyl-3,3,4-trichlorobutyl, 1-bromo-2-butenyl;
(2) X^{d} represents —C(═O)CR^{8b}R^{9b}R^{10c},
   R^{1b}, R^{3b} and R^{8b} each represent hydrogen,
   R^{2b}, R^{2c} and R^{9b} each represent methyl,
   R^{3c} and R^{4a} each represent acetyl,
   R^{10c} represents 2-butenyl or 2-methyl-3,3,4-trichlorobutyl;
(3) X^{d} represents —C(═O)CR^{8c}R^{9c}R^{10d},
   R^{1b}, R^{3b}, R^{3c}, R^{4a} and R^{8a} each represent hydrogen,
   R^{2b} and R^{2c} each represent methyl,
   R^{9c} represents ethyl,
   R^{10d} represents n-butyl;
(4) X^{d} represents —C(═O)CR^{8d}R^{9d}R^{10e},
   R^{1b}, R^{3b}, R^{3c}, R^{4a} and R^{8a} each represent hydrogen,
   R^{2b} and R^{2c} each represent methyl,
   R^{9d} and R^{10e} together with the carbon atom to which they are attached form an unsubstituted cyclohexyl ring;
(5) X^{d} represents unsubstituted —C(═O)phenyl, —C(═O)-4-chlorophenyl or —C(═O)-3-phenoxyphenyl,
   R^{1b}, R^{3b}, R^{3c} and R^{4a} each represent hydrogen and
   R^{2b} and R^{2c} each represent methyl;
(6) X^{d} represents —C(═O)OR⁵c or —C(═O)NR^{6a}R^{7a},
   R^{1b}, R^{3b}, R^{3c} and R^{4a} each represent hydrogen,
   R^{2b} represents hydrogen, methyl or ethyl,
   R^{2c} represents methyl or ethyl,
   R^{3b} represents hydrogen,
   R^{3c} and R^{4d} both simultaneously represent hydrogen,
   R^{5c} represents hydrogen or represents C₁-C₆-alkyl which is optionally mono- or polysubstituted by identical or different substituents,
   R^{6a} represents hydrogen, methyl, ethyl, n- or isopropyl, n-, iso-, sec- or tert-butyl, pentyl or hexyl, represents in particular hydrogen or methyl,
   R^{7a} represents in each case optionally phenyl-substituted methyl or propyl (in particular 2-phenylpropyl) or represents -M^{d}-Q^{a}-Z^{a},
   M^{a} represents phenylene which is mono- or polysubstituted by R^{20a}, represents in particular the cycles M-1, M-2 and M-3,
   R^{20a} represents hydrogen, fluorine, chlorine, bromine, C₁-C₄-alkyl, C₁-C₄ alkylthio, C₁-C₄-haloalkyl, C₁-C₄-haloalkoxy,
   Q^{a} represents a direct bond, —CH₂—, —(CH₂)₂—, —(CH₂)₃—, —OCH₂—, —O(CH₂)₂—, —O(CH₂)₃—, O, S or NR^{12a},
   R^{12a} represents hydrogen, methyl, ethyl, n- or isopropyl, n-, sec-, iso- or tert-butyl,
   Z^{a} represents phenyl or pyridyl, optionally mono- to pentasubstituted by identical or different substituents, where the substituents are selected from the list W^{1a},
   W^{1a} represents fluorine, chlorine, bromine, cyano, nitro, formyl, methyl, ethyl, n- or i-propyl, n-, s- or t-butyl, methoxy, ethoxy, n- or i-propoxy, trifluoromethyl, trifluoroethyl, difluoromethoxy, trifluoromethoxy, difluorochlormethoxy or trifluoroethoxy, in each case doubly attached difluoromethylenedioxy or tetrafluoroethylenedioxy.

Preferred radical definitions of the formulae mentioned above and below are given below. These definitions apply to the end products and likewise to all intermediates.

Preference is given to preparing, by the process according to the invention, those compounds of the formulae (I) and (II) in which all radicals have in each case the preferred meanings mentioned above.

Particular preference is given to preparing, by the process according to the invention, those compounds of the formulae (I) and (II) in which all radicals have in each case the particularly preferred meanings mentioned above.

Very particular preference is given to preparing, by the process according to the invention, those compounds of the formulae (I) and (II) in which all radicals have in each case the very particularly preferred meanings mentioned above.

Preference is given to those compounds of the formulae (I-k) and (II-b), in which all radicals have in each case the preferred meanings mentioned above.

Particular preference is given to those compounds of the formulae (I-k) and (II-b), in which all radicals have in each case the particularly preferred meanings mentioned above.

Very particular preference is given to those compounds of the formulae (I-k) and (II-b), in which all radicals have in each case the very particularly preferred meanings mentioned above.

However, the general or preferred radical definitions or illustrations listed above can also be combined with one another, i.e. between their respective ranges and preferred ranges, as desired.

They apply both to the end products and, correspondingly, to the precursors and intermediates.

In the definitions of the symbols giving in the formulae above, collective terms were used which are generally representative for the following substituents:

Halogen: fluorine, chlorine, bromine and iodine;

Alkyl: saturated straight-chain or branched hydrocarbon radicals having 1 to 8 carbon atoms, for example C₁-C₆-alkyl such as methyl, ethyl, propyl, 1-methylethyl, butyl, 1-methylpropyl, 2-methyl-propyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; heptyl, octyl.

Haloalkyl: straight-chain or branched alkyl groups having 1 to 8 carbon atoms (as mentioned above), where in these groups some or all of the hydrogen atoms may be replaced by halogen atoms as mentioned above, for example C₁-C₃-haloalkyl such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromethyl, 1-fluoroethyl, 2-fluoroethyl, 2,2-difluoroethyl, 2,2,2-trifluoroethyl, 2-chloro-2-fluoroethyl, 2-chloro-2-difluoroethyl, 2,2-dichloro-2-fluoroethyl, 2,2,2-trichloroethyl, pentafluoroethyl and 1,1,1-trifluoroprop-2-yl.

—SiR¹⁵R¹⁶R¹⁷: —SiMe₃, —SiMe₂Et, —SiMe₂CHMe₂, —SiMe₂CH₂CHMe₂, —SiMe₂CH₂CMe₃, —SiMe₂OCHMe₂, —SiMe₂OCH₂CHMe₂, —SiMe₂OMe, —SiMe₂CMe₃, —SiMe₂CH₂CH₂Me.

Alkenyl: unsaturated straight-chain or branched hydrocarbon radicals having 2 to 8 carbon atoms and a double bond in any position, for example C₂-C₆-alkenyl such as ethenyl, 1-propenyl, 2-propenyl, 1-methylethenyl, 1-butenyl, 2-butenyl, 3-butenyl, 1-methyl-l-propenyl, 2-methyl-l-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1,-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

Alkynyl: straight-chain or branched hydrocarbon groups having 2 to 8 carbon atoms and a triple bond in any position, for example C₂-C₆-alkynyl such as ethynyl, 1-propynyl, 2-propynyl, 1-butinyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl.

Cycloalkyl: monocyclic saturated hydrocarbon groups having 3 to 8 carbon ring members, such as cyclopropyl, cyclobutyl, cyclopentyl, cyclohexyl, cycloheptyl and cyclooctyl.

Cycloalkenyl: monocyclic non-aromatic hydrocarbon groups having 3 to 8 carbon ring members and at least one double bond, such as cyclopenten-1-yl, cyclohexen-1-yl, cyclohepta-1,3-dien-1-yl.

Alkoxycarbonyl: an alkoxy group having 1 to 6 carbon atoms (as mentioned above) which is attached to the skeleton via a carbonyl group (—CO—).

Aryl: an unsubstituted or substituted cyclic aromatic hydrocarbon radical having 6 to 14 ring members: for example phenyl, naphthyl, anthracyl, preferably phenyl, naphthyl, particularly preferably phenyl.

Heterocyclyl/hetaryl: an unsubstituted or substituted unsaturated or fully or partially saturated heterocyclic 5- to 7-membered ring or an unsaturated or fully or partially saturated heterocyclic 3- to 8-membered ring which contains up to 4 nitrogen atoms or alternatively 1 nitrogen atom and up to 2 further heteroatoms selected from the group consisting of N, O and S: for example oxiranyl, aziridinyl, 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,3-dihydrothien-2-yl, 2,3-dihydrothien-3-yl, 2,4-di-hydrothien-2-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl, 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl, 1-pyrrolyl, 1-pyrazolyl, 1,2,4-triazol-1-yl, 1-imidazolyl, 1,2,3-triazol-1-yl, 1,3,4-triazol-1-yl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

Processes and Intermediates

Process (1)

Using, for example, methoxymalonoyl dichloride and N-(4-methoxybenzyl)-3-oxo-3-phenyl-propanamide as starting materials and a Meerwein salt as alkylating agent, the course of the process (1) according to the invention can be illustrated by the reaction equation below:

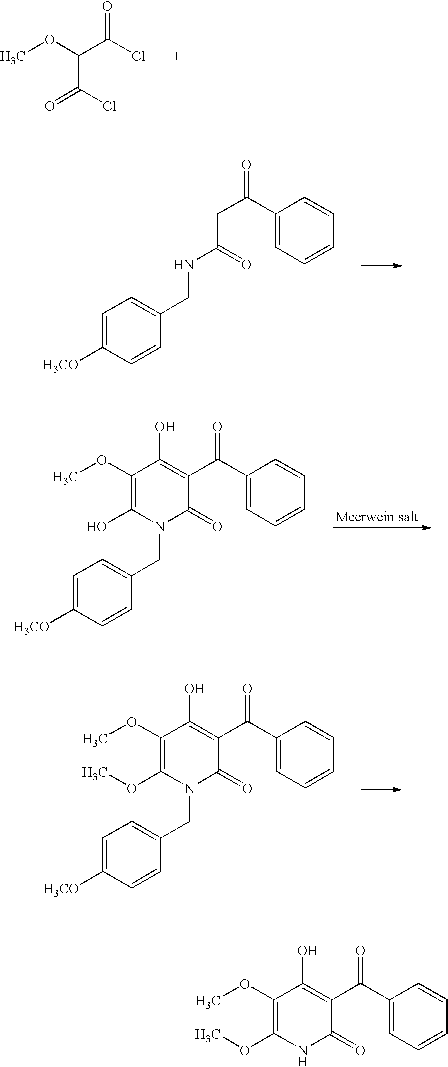

Step (a)

The formula (III) provides a general definition of the oxymalonic acid derivatives required as starting materials for carrying out step (a) of the process according to the invention. In this formula, Y preferably represents fluorine, chlorine, bromine, hydroxy, —O—Li, —O—Na, —O—K, —O—Cs, particularly preferably chlorine, bromine, hydroxy or —O—Li, very particularly preferably chlorine, hydroxy or —O—Li, especially preferably chlorine. The acid halides can be prepared in particular in situ from the corresponding acid or the relevant salt. Preferably, the chloride (Y=chlorine) is prepared from the oxymalonic acid derivative where Y=hydroxy or —O—Li.

Some of the oxymalonic acid derivatives of the formula (III) are known (c.f. J. Chem. Soc. Perkin Trans. I 1985, 1757-1766 or U.S. Pat. No. 5,889,184).

Oxymalonic acid derivatives of the formula (III-a)

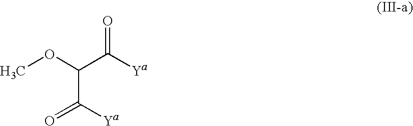

in which Y^{a} represents fluorine, bromine, —O—Li or —O—Cs are novel.

Oxymalonic acid derivatives of the formula (III-a) can be prepared by standard methods of organic synthesis, if appropriate also in situ.

The formula (IV) provides a general definition of the amides furthermore required as starting materials for carrying out step (a) of the process according to the invention. In this formula X^{a} preferably, particularly preferably, very particularly preferably and especially preferably has those meanings which have already been given in connection with the description of the compounds of the formulae (I) and (II) which can be prepared according to the invention as being preferred, particularly preferred, etc., for the radical X, where in each case X^{a} does not represent hydrogen, —C(═O)OH or —C(═O)NR⁶R⁷. R^{1a} preferably, particularly preferably, very particularly preferably and especially preferably has those meanings which have been given for the radical R¹ as being preferred, particularly preferred, etc., where in each case R^{1a} does not represent hydrogen. R^{5a} preferably, particularly preferably, very particularly preferably and especially preferably has those meanings which have been given as being preferred, particularly preferred, etc., for the radical R⁵, where in each case R^{5a} does not represent hydrogen.

Amides of the formula (IV) are known and/or can be obtained by known processes.

Amides of the formula (IV) in which X^{a} represents —C(═O)R⁸R⁹R¹⁰, —C(═O)aryl or —C(═O)hetaryl can be prepared, for example, by reacting
(h) carboxylic acid derivatives of the formula (V)

HO—X^{a} (V)
with Meldrum' s acid of the formula

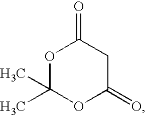

if appropriate in the presence of a diluent (for example dichloromethane), if appropriate in the presence of a condensing agent (for example DCC), at temperatures of, for example, between room temperature and 110° C., followed by reaction with an amine of the formula (VI)

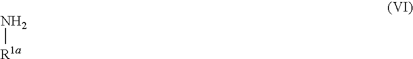

in which R^{1a} has the meanings given above (cf. J. Comb. Chem. 2002, 4, 470-474).

Amides of the formula (IV), in which X^{a} represents —C(═O)OR^{5a} can be prepared, for example, by reacting
(i) malonic monoester monochlorides of the formula (VII)

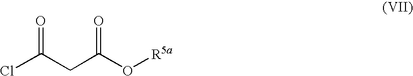

in which R^{5a} has the meanings given above,
with an amine of the formula (VI)

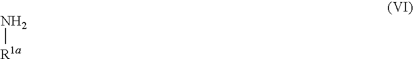

in which R^{1a} has the meanings given above,
if appropriate in the presence of a diluent (for example dichloromethane), if appropriate in the presence of an acid binder (for example triethylamine), at temperatures of, for example, between 0° C. and room temperature (c.f. Tetrahedron: Asymmetry 2006, 17(4), 642-657).

The starting materials for the process (h) and (i) are known and/or can be obtained by known processes.

In general, it is advantageous to carry out step (a) of the preparation process according to the invention in the presence of diluents, if appropriate, and in the presence of basic reaction auxiliaries, if appropriate. Preferably, step (a) of the process according to the invention is carried out without using bases.

Diluents are advantageously employed in such an amount that the reaction mixture remains readily stirrable during the entire process.

Suitable diluents for carrying out step (a) of the process according to the invention are all inert organic solvents. These preferably include aliphatic, alicyclic or aromatic hydrocarbons, such as, for example, petroleum ether, pentane, hexane, heptane, octane, nonane and industrial hydrocarbons; for example white spirits with components having boiling points in the range of, for example, from 40° C. to 250° C., cymene, benzine fractions within a boiling point interval of from 70° C. to 190° C., cyclohexane, methylcyclohexane, benzene, toluene, xylene or decalin; halogenated hydrocarbons such as, for example, chlorobenzene, dichlorobenzene, trichlorobenzene, chlorotoluene, dichloromethane, chloroform, carbon tetrachloride, 1,2-dichloroethane, trichloroethane, tetrachloroethane or tetrachloroethylene; ethers, such as diethyl ether, diisopropyl ether, methyl t-butyl ether, methyl t-amyl ether, dioxane, tetrahydrofuran, 1,2-dimethoxyethane, 1,2-diethoxyethane or anisol; nitrogen hydrocarbons, such as nitromethane, nitroethane, nitropropane, nitrobenzene, chloronitrobenzene, o-nitrotoluene; nitriles, such as acetonitrile, propionitrile, n- or i-butyronitrile or benzonitrile, m-chlorobenzonitrile; amides, such as N,N-dimethylformamide, N,N-dimethylacetamide, N-methylformanilide, N-methylpyrrolidone or hexamethylphosphoric triamide, and also dimethyl sulphoxide. It is also possible to use mixtures of the solvents and diluents mentioned.

Particularly preferred diluents for carrying out step (a) of the process according to the invention are halogenated hydrocarbons, such as, for example, chlorobenzene, dichlorobenzene, trichlorobenzene, chlorotoluene, dichloromethane, chloroform, carbon tetrachloride, 1,2-dichloroethane, trichloroethane, tetrachloroethane or tetrachloroethylene, and also aliphatic, alicyclic or aromatic hydrocarbons, such as, for example, petroleum ether, pentane, hexane, heptane, octane, nonane and industrial hydrocarbons; for example white spirits with components having boiling points in the range of, for example, from 40° C. to 250° C., cymene, benzine fractions within a boiling point interval of from 70° C. to 190° C., cyclohexane, methylcyclohexane, benzene, toluene, xylene or decalin.

Very particular preference is given to using toluene or dichloromethane or a mixture of dichloromethane and toluene.

When carrying out step (a) of the process according to the invention, the reaction temperatures can be varied within a relatively wide range. In general, the process is carried out at temperatures of from −50° C. to +150° C., preferably at temperatures of from 0° C. to 110° C., particularly preferably at from 20° C. to 30° C., very particularly preferably at room temperature.

For carrying out step (a) of the process according to the invention for preparing the compounds of the formula (I-b), in general from 0.5 to 5 mol, preferably from 1 to 2 mol, of oxymalonic acid derivatives of the formula (III) are employed per mole of the amide of the formula (IV). The reaction time is from 12 to 60 hours. Work-up is carried out by customary methods.

Step (b)

The formula (I-b) provides a general definition of the 2-pyridones required as starting materials for carrying out step (b) of the process according to the invention. In this formula, X^{a} preferably, particularly preferably, very particularly preferably and especially preferably has those meanings which have already been given in connection with the description of the compounds of the formulae (I) and (II) which can be prepared according to the invention as being preferred, particularly preferred, etc., for the radical X, where in each case X^{a} does not represent hydrogen, —C(═O)OH or —C(═O)NR⁶R⁷. R^{1a} preferably, particularly preferably, very particularly preferably and especially preferably has those meanings which have been given as being preferred, particularly preferred, etc., for the radical R¹, where in each case R^{1a} does not represent hydrogen. R^{2a} preferably represents methyl. R^{2a} also preferably represents ethyl, particularly preferably methyl.

The 2-pyridones of the formula (I-b) are a subset of the 2-pyridones of the formula (I). As far as they are comprised by the formula (I-k), they are novel. They can be prepared by step (I-k) of the process according to the invention.

The following alkylating agents are suitable for carrying out step (b) of the process according to the invention: diazomethane, trimethylsilyldiazomethane (TMS-diazomethane) or trialkyloxonium salts. Preference is given to using trimethyloxonium or triethyloxonium salts, which may comprise PF6⁻, SbF₆⁻, SbCl₆⁻ or BF₄⁻ as counterions. These alkylating agents are known.

In general, it is advantageous to carry out step (b) of the preparation process according to the invention in the presence of diluents, if appropriate, and in the presence of reaction auxiliaries, if appropriate.

Diluents are advantageously employed in such an amount that the reaction mixture remains readily stirrable during the entire process.

If the alkylating agent used is diazomethane or TMS-diazomethane, when carrying out step (b) of the process according to the invention use is preferably made of the following solvents: alcohols, such as, for example, methanol, ethanol or isopropanol; aliphatic, alicyclic or aromatic hydrocarbons, such as, for example, petroleum ether, pentane, hexane, heptane, octane, nonane and industrial hydrocarbons; for example white spirits with components having boiling points in the range of, for example, from 40° C. to 250° C., cymene, benzine fractions within a boiling point interval of from 70° C. to 190° C., cyclohexane, methylcyclohexane, benzene, toluene, xylene or decalin; halogenated hydrocarbons, such as chlorobenzene, dichlorobenzene, trichlorobenzene, chlorotoluene, dichloromethane, chloroform, carbon tetrachloride, 1,2-dichloroethane, trichloroethane, tetrachloroethane or tetrachloroethylene; ethers, such as diethyl ether, diisopropyl ether, methyl t-butyl ether, methyl t-amyl ether, dioxane, tetrahydrofuran, 1,2-dimethoxyethane, 1,2-diethoxyethane or anisol; esters, such as methyl acetate or ethyl acetate; ketones, such as acetone, butanone, methyl isobutyl ketone or cyclohexanone; nitriles, such as acetonitrile, propionitrile, n- or i-butyronitrile or benzonitrile, m-chlorobenzonitrile. It is also possible to use mixtures of the solvents and diluents mentioned.

If the alkylating agent used is diazomethane or TMS-diazomethane, when carrying out step (b) of the process according to the invention use is with preference made of the following solvents: alcohols, such as, for example, methanol or ethanol; halogenated hydrocarbons, such as, for example, chlorobenzene, dichloromethane, chloroform, carbon tetrachloride, 1,2-dichloroethane, trichloroethane, tetrachloroethane or tetrachloroethylene, and also ethers, such as diethyl ether, diisopropyl ether, methyl t-butyl ether, methyl t-amyl ether, dioxane, tetrahydrofuran, 1,2-dimethoxyethane or 1,2-diethoxyethane.

When using diazomethane or TMS-diazomethane as alkylating agent, step (b) of the process according to the invention is, if appropriate, carried out in the presence of a suitable acid acceptor. Suitable acid acceptors are customary inorganic or organic bases. These preferably include alkali metal carbonates of bicarbonates, such as, for example, lithium carbonate, sodium carbonate, potassium carbonate, caesium carbonate, potassium bicarbonate or sodium bicarbonate; tertiary amines, such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU), and also proton sponge (1,8-bisdimethylaminonaphthatene).

If the alkylating agent used is trialkyloxonium salts, when carrying out step (b) of the process according to the invention use is preferably made of the following solvents: aromatic hydrocarbons, such as, for example, benzene, toluene, xylene; halogenated hydrocarbons, such as, for example, chlorobenzene, dichlorobenzene, trichlorobenzene, chlorotoluene, dichloromethane, chloroform, carbon tetrachloride, 1,2-dichloroethane, trichloroethane, tetrachloroethane or tetrachloroethylene; ethers, such as diethyl ether, diisopropyl ether, methyl t-butyl ether, methyl t-amyl ether, dioxane, tetrahydrofuran, 1,2-dimethoxyethane, 1,2-diethoxyethane or anisol; esters, such as methyl acetate or ethyl acetate; ketones, such as acetone, butanone, methyl isobutyl ketone or cyclohexanone; nitriles, such as acetonitrile, propionitrile, n- or i-butyronitrile or benzonitrile, m-chlorobenzonitrile, nitro hydrocarbons, such as nitromethane, nitroethane, nitropropane, nitrobenzene, chloronitrobenzene, o-nitrotoluene. It is also possible to use mixtures of the solvents and diluents mentioned.

If the alkylating agent used is trialkyloxonium salts, when carrying out step (b) of the process according to the invention use is with preference made of the following diluents: halogenated hydrocarbons, such as, for example, chlorobenzene, dichlorobenzene, dichloromethane, chloroform, carbon tetrachloride, 1,2-dichloroethane, trichloroethane, tetrachloroethane or tetrachloroethylene.

When using trialkyloxonium salts as alkylating agents, step (b) of the process according to the invention is, if appropriate, carried out in the presence of a suitable acid acceptor. Suitable acid acceptors are customary inorganic or organic bases. These preferably include alkali metal carbonates or bicarbonates, such as, for example, lithium carbonate, sodium carbonate, potassium carbonate, caesium carbonate, sodium bicarbonate, potassium bicarbonate; tertiary amines, such as trimethylamine, triethylamine, tributylamine, diisopropylethylamine, N,N-dimethylaniline, N,N-dimethylbenzylamine, pyridine, N-methylpiperidine, N-methylmorpholine, N,N-dimethylaminopyridine, 2,6-di-tert-butylpyridine, diazabicyclooctane (DABCO), diazabicyclononene (DBN) or diazabicycloundecene (DBU), and also proton sponge (1,8-bisdimethylaminonaphthalene). Preference is given to using tertiary amines, such as triethylamine and diisopropylethylamine.

If appropriate, the reaction can be carried out in the presence of molecular sieves of a suitable pore size (3 Å, 4 Å, 5 Å) and, if appropriate, in the presence of boron trifluoride etherate.

When carrying out step (b) of the process according to the invention, the reaction temperatures can be varied within a relatively wide range. In general, the reaction is carried out at temperatures of from −78° C. to +150° C., preferably at temperatures of from −78° C. to +50° C., very particularly preferably at from 0° C. to 30° C.

For carrying out step (b) of the process according to the invention for preparing 2-pyridones of the formula (I-c), in general from 0.5 to 20 mol, preferably from 1 to 5 mol, of alkylating agent and from 0 to 20 mol, preferably from 1 to 5 mol, of acid acceptor are employed per mole of the 2-pyridone of the formula (I-b). The reaction time is from 1 to 48 hours. The reaction is preferably carried out under an atmosphere of protective gas, such as nitrogen or argon. Work-up is carried out by customary methods.

Step (c)

The 2-pyridones of the formula (I-b) which can be used as starting materials for carrying out step (c) of the process according to the invention have already been described above.

The formula (I-c) provides a general definition of the 2-pyridones required as starting materials for carrying out step (c) of the process according to the invention. In this formula, X^{a} preferably, particularly preferably, very particularly preferably and especially preferably has those meanings which have already been given in connection with the description of the compounds of the formulae (I) and (II) which can be prepared according to the invention as being preferred, particularly preferred, etc., for the radical X, where in each case X^{a} does not represent hydrogen, —C(═O)OH or —C(═O)NR⁶R⁷. R^{1a} preferably, particularly preferably, very particularly preferably and especially preferably has those meanings which have been given as being preferred, particularly preferred, etc., for the radical R¹, where in each case R^{1a} does not represent hydrogen. R^{2a} preferably represents methyl. R^{2a} also preferably represents ethyl, particularly preferably methyl.

The 2-pyridones of the formula (I-c) are a subset of the 2-pyridones of the formula (I). As far as they are comprised by the formula (I-k), they are novel. They can be prepared by step (b) of the process according to the invention.

2-pyridones of the formula (I-c-1),

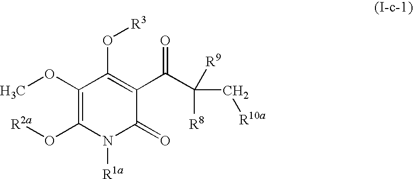

in which
R⁸ and R⁹ have the meanings given above and
R^{10a} represents hydrogen or represents alkyl, alkenyl, alkynyl, cycloalkyl, cycloalkylalkyl, cycloalkenyl, cycloalkenylalkyl, bicycloalkyl, bicycloalkylalkyl, aryl, arylalkyl, hetaryl or hetarylalkyl, each of which is optionally mono- or polysubstituted by identical or different substituents,

can also be prepared by reacting
(k) per mole of 2-pyridone of the formula (I-l)

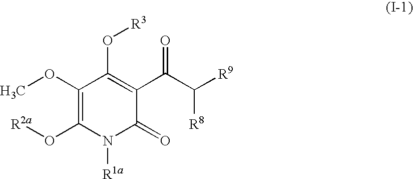

in which R^{1a}, R^{2a}, R³, R⁸ and R⁹ have the meanings given above,
from 1 to 5 mol, preferably from 2 to 3 mol, of a base (for example lithium hexamethyldisilazide)
and from 1 to 5 mol, preferably from 2 to 3 mol, of a compound of the formula (IX)

LG-CH₂—R^{12a} (IX)
in which
R^{10a} has the meanings given above and
LG represents halogen, tosylate or triflate,
followed by work-up by customary methods.

The formula (I-l) provides a general definition of the 2-pyridones likewise required as starting materials for carrying out the process (k) according to the invention. In this formula R^{2a}, R³, R⁸ and R⁹ preferably, particularly preferably, very particularly preferably and especially preferably have those meanings which have already been given in connection with the description of the compounds of the formulae (I) and (II) which can be prepared according to the invention as being preferred, particularly preferred, etc., for these radicals. R^{a} preferably, particularly preferably, very particularly preferably and especially preferably has those meanings which have already been given as being preferred, particularly preferred, etc., for the radical R¹, where in each case R^{1a} does not represent hydrogen.

The 2-pyridones of the formula (I-l) are a subset of the 2-pyridones of the formula (I-c). As far as they are comprised by the formula (I-k), they are novel. They can be prepared by step (b) of the process according to the invention.

The formula (IX) provides a general definition of the compounds likewise required as starting materials for carrying out the process (k) according to the invention. In this formula LG preferably represents chlorine, bromine, iodine, tosylate or triflate.
R^{10a} preferably represents hydrogen,
   represents in each case straight-chain or branched C₁-C₁₁-alkyl, C₂-C₁₁-alkenyl or C₂-C₁₁-alkynyl, each of which is optionally mono- or polysubstituted by identical or different substituents from the group consisting of halogen, C₁-C₄-alkoxy, C₁-C₄-alkoxycarbonyl, C₃-C₇-cycloalkyl (which for its part may be substituted by halogen, C₁-C₄-alkyl or C₁-C₄-alkoxy), phenyl, benzyloxy (which for their part may in each case be substituted by halogen, cyano, C₁-C₄-alkyl, C₁-C₄-haloalkyl, C₁-C₄-haloalkoxy having in each case 1 to 9 fluorine, chlorine and/or bromine atoms, phenyl, phenoxy, hetaryl or hetaryloxy, where the latter phenyl, phenoxy, hetaryl or hetaryloxy substituents for their part may optionally be substituted by halogen, cyano, nitro, C₁-C₄-alkyl, C₁-C₄-haloalkyl, C₁-C₄-haloalkoxy having in each case 1 to 9 fluorine, chlorine and/or bromine atoms) or hetaryl (which for its part may be substituted by halogen, cyano, nitro, C₁-C₄-alkyl, C₁-C₄-haloalkyl, C₁-C₄-haloalkoxy having in each case 1 to 9 fluorine, chlorine and/or bromine atoms, phenyl, phenoxy, hetaryl or hetaryloxy, where the latter phenyl, phenoxy, hetaryl or hetaryloxy substituents for their part may be substituted by halogen, cyano, nitro, C₁-C₄-alkyl, C₁-C₄-haloalkyl, C₁-C₄-haloalkoxy having in each case 1 to 9 fluorine, chlorine, and/or bromine atoms),
   represents cycloalkyl, cycloalkenyl, phenyl or hetaryl, optionally mono- or polysubstituted by identical or different substituents from the group consisting of halogen, C₁-C₄-alkyl, C₁-C₄-haloalkyl having 1 to 9 fluorine, chlorine and/or bromine atoms, phenyl, phenoxy (which for their part may in each case be substituted by halogen, cyano, nitro, C₁-C₄-alkyl, C₁-C₄-haloalkyl, C₁-C₄-haloalkoxy having in each case 1 to 9 fluorine, chlorine and/or bromine atoms).
   R^{10a} particularly preferably represents hydrogen,
   represents C₁-C₉-alkyl, C₂-C₉-alkenyl or C₂-C₉-alkynyl, each of which is optionally mono- to tetrasubstituted by identical or different substituents from the group consisting of halogen, methoxy, ethoxy, methoxycarbonyl, ethoxycarbonyl, cyclopentyl, cyclohexyl, cycloheptyl (where cyclopentyl, cyclohexyl and cycloheptyl for their part may be substituted by methyl, ethyl, i-propyl), phenyl, benzyloxy (which for their part may in each case be substituted by fluorine, chlorine, bromine, methyl, ethyl, n-, i-propyl, n-, s-, t-butyl, trifluoromethyl),
   represents cyclohexyl, cyclohexenyl, phenyl, thienyl, isoxazolyl or pyridinyl, optionally mono- to tetrasubstituted by identical or different substituents from the group consisting of fluorine, chlorine, bromine, methyl, ethyl, n-, i-propyl, n-, s-, t-butyl, trifluoromethyl, phenyl, phenoxy, chlorophenoxy, dichlorophenoxy, chlorotrifluoromethylphenoxy.

In general, it is advantageous to carry out step (c) of the preparation process according to the invention in the presence of diluents, if appropriate, and in the presence of reaction auxiliaries, if appropriate.

Diluents are advantageously employed in such an amount that the reaction mixture remains readily stirrable during the entire process.

The use of protective groups at nitrogen atoms and their removal has been described in a general manner (cf. Protective Groups in Organic Synthesis, 4th Ed., Th. W. Greene, P. G. M. Wuts, Eds., John Wiley & Sons, Inc., New York, 2006; Protecting Groups, 3rd Ed., Ph. J. Kocienski, Ed., Georg Thieme Verlag, Stuttgart, N.Y., 2003). The removal of protective groups at 2-pyridones or analogous systems is known (c.f. Bioorg. Med. Chem. Lett. 2006, 16, 5668-5672). Preference is given to protective groups which can be removed under mild conditions. These include in particular, but not exclusively, N-allyl-, N-benzyl and substituted N-benzyl protective groups.

Step (c) of the preparation process according to the invention can be carried out, for example, but not exclusively, by hydrogenation in the presence of suitable catalysts, using suitable acidic reaction auxiliaries, such as strong Brønsted or Lewis acids, or using transition metal catalysts.

Suitable catalysts for carrying out the catalytic hydrogenation are all customary hydrogenation catalysts, such as, for example, platinum catalysts (for example platinum plate, platinum sponge, platinum black, colloidal platinum, platinum oxide, platinum wire), palladium catalysts (for example palladium sponge, palladium black palladium oxide, palladium/carbon, colloidal palladium, palladium-barium sulphate, palladium-barium carbonate, palladium-hydroxide), nickel catalysts (fore example reduced nickel, nickel oxide, Raney-nickel). However, preference is given to using noble metal catalysts, such as, for example, platinum and palladium catalysts, if appropriate on a suitable support, such as, for example, on carbon, particularly preferably Pd/C.

Suitable solvents for the hydrogenation reaction are in each case all customary inert organic solvents. Preference is given to using alcohols, such as methanol or ethanol, nitriles, such as acetonitrile, propionitrile, n- or i-butyronitrile or benzonitrile; amides, such as N,N-dimethylformamide, N,N-di-methylacetamide, N-methylformanilide, N-methylpyrrolidone or hexamethylphosphoric triamide; ethers, such as diethyl ether, diisopropyl ether, methyl t-butyl ether, methyl t-amyl ether, dioxane, tetrahydrofuran, 1,2-dimethoxyethane, 1,2-diethoxyethane or anisol; esters, such as methyl acetate or ethyl acetate; sulphoxides, such as dimethyl sulphoxide; sulphones, such as sulpholane; organic acids, such as formic acid or acetic acid; or water. It is also possible to use mixtures of the solvents and diluents mentioned. Particular preference is given to using alcohols, such as methanol or ethanol and ethers such as dioxane and tetrahydrofuran, and also mixtures thereof.

Suitable acidic reaction auxiliaries for carrying out step (c) of the preparation process according to the invention are mineral acids, organic acids or Lewis acids. The mineral acids include hydrohalic acids, such as hydrofluoric acid, hydrochloric acid, hydrobromic acid or hydroiodic acid, and also sulphuric acid, phosphoric acid, phosphorous acid, nitric acid, and the Lewis acids include, for example, aluminium(III) chloride, boron trifluoride or else etherate, titanium(V) chloride, tin(V) chloride. The organic acids include, for example, formic acid, acetic acid, trifluoroacetic acid, propionic acid, malonic acid, lactic acid, oxalic acid, fumaric acid, adipic acid, steric acid, tartaric acid, oleic acid, methanesulphonic acid, benzoic acid, benzenesulphonic acid or p-toluenesulphonic acid. Particular preference is given to using trifluoroacetic acid.

When carrying out step (c) of the process according to the invention, the reaction temperatures can be varied within a relatively wide range. In general, the process is carried out at temperatures of from −78° C. to +150° C., preferably at temperatures of from 0° C. to +110° C.

For carrying out step (c) of the process according to the invention for preparing the compounds of the formula (I), the 2-pyridone of the formula (I-b) or (I-c) is generally hydrogenated in the presence of from 0.1 to 50% by weight, preferably from 0.5 to 10% by weight, transition metal catalyst. The reaction time is from 1 to 48 hours. In principle, the reaction can be carried out under atmospheric pressure. Preferably, the reaction is carried out under atmospheric pressure or at pressures of up to 15 bar in an atmosphere of hydrogen.

Alternatively, when carrying out step (c) of the process according to the invention for preparing the compounds of the formula (I), generally from 1 to 10 mol, preferably from 1 to 5 mol, of Lewis acid are employed per mole of the 2-pyridone of the formula (I-b) or (I-c). Mineral acids or organic acids are used as solvent. The reaction time is from 1 to 48 hours. The reaction is carried out at temperatures of from 0° C. to 120° C., preferably at from 20° C. to 80° C. Work-up is carried out by customary methods.

The formula (I-d) provides a general definition of the 2-pyridones obtained when carrying out step (c) of the process according to the invention. In this formula, X^{a} preferably, particularly preferably, very particularly preferably and especially preferably has those meanings which have already been given in connection with the description of the compounds of the formulae (I) and (II) which can be prepared according to the invention as being preferred, particularly preferred, etc., for the radical X, where in each case X^{a} does not represent hydrogen, —C(═O)OH and —C(═O)NR⁶R⁷. R² and R³ have preferably, particularly preferably, very particularly preferably and especially preferably those meanings which have already been mentioned in connection with the description of the compounds of the formulae (I) and (II) which can be prepared according to the invention as being preferred, particularly preferred, etc., for these radicals.

The 2-pyridones of the formula (I-d) are a subset of the 2-pyridones of the formula (I). As far as they are comprised by the formula (I-k), they are novel.

Step (d)

The formulae (I-b), (I-c) and (I-d) provide general definitions of the 2-pyridones required as starting materials for carrying out step (d) of the process according to the invention. In this formula, X^{a} always represents —C(═O)OR^{5a}, where R^{5a} preferably, particularly preferably, very particularly preferably and especially preferably has the meanings given above.

The cleavage in step (d) of the process according to the invention is carried out by customary methods. For example, from 1 to 5 mol, preferably from 1 to 3 mol, of a hydroxide base (for example lithium hydroxide) are reacted per mole of 2-pyridone of the formula (I-b) or the formula (I-c) or the formula (I-d), in which X^{a} represents —C(═O)OR^{5a}, in the presence of a diluent (for example a mixture of tetrahydrofuran and water), followed by work-up by customary methods.

Process (2)

Using, for example, methyl 4-hydroxy-5,6-dimethoxy-1-(4-methoxybenzyl)-2-oxo-1,2-dihydropyridine-3-carboxylate and 2-(1,3-dimethylbutyl)aniline as starting materials, the course of the process (2) according to the invention can be illustrated by way of the following reaction equation:

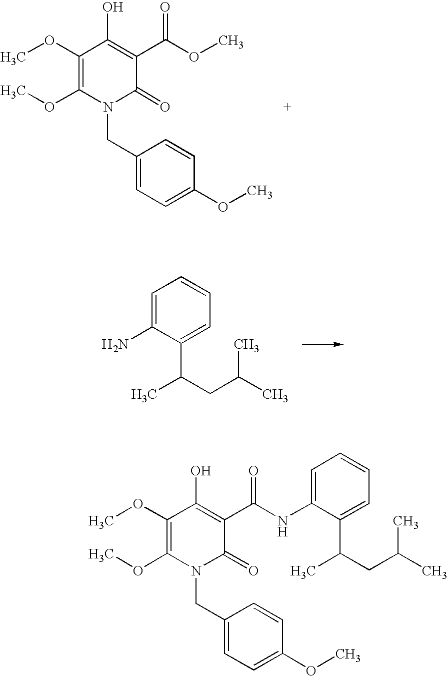

Step (e)

The formula (I-f) provides a general definition of the 2-pyridones required as starting materials for carrying out step (e) of the process according to the invention. In this formula R¹, R² and R³ preferably, particularly preferably, very particularly preferably and especially preferably have those meanings which have already been given in connection with the description of the compounds of the formulae (I) and (II) which can be prepared according to the invention as being preferred, particularly preferred, etc., for these radicals. X^{b} represents —C(═O)OH or —C(═O)OR^{5b}, where R^{5b} preferably represents methyl, ethyl or n-propyl. X^{b} particularly preferably represents —C(═O)OH or —C(═O)OCH₃.

The 2-pyridones of the formula (I-f) are a subset of the 2-pyridones of the formula (I). As far as they are comprised by the formula (I-k), they are novel. They can be prepared by process (1) according to the invention.

Using customary methods, it is also possible to convert 2-pyridones of the formula (I-f), in which X^{b} represents —C(═O)OR^{5b} into 2-pyridones of the formula (I-f), in which X^{b} represents —C(═O)OH. To this end, for example, from 1 to 5 mol, preferably from 1 to 3 mol, of a hydroxide base (for example lithium hydroxide) are reacted per mole of 2-pyridone of the formula (I-f) in which X^{b} represents —C(═O)OR^{5b}, in presence of a diluent (for example a mixture of tetrahydrofuran and water), followed by work-up according to customary methods.

The formula (VIII) provides a general definition of the amines furthermore required as starting materials for carrying out step (e) of the process according to the invention. In this formula R⁶ and R⁷ preferably, particularly preferably, very particularly preferably and especially preferably have those meanings which have already been given in connection with the description of the compounds of the formulae (I) and (II) which can be prepared according to the invention as being preferred, particularly preferred, etc., for these radicals.

Amines of the formula (VIII) are known and/or can be prepared by known processes.

In principle, a large number of different methods are known for preparing carboxamides from carboxylic acids and carboxylic esters (cf., for example, G. Benz in Comprehensive Organic Synthesis, 1^{st} Ed., Pergamon Press, Oxford, 1991, Vol. 6, pp. 381-417; P. D. Bailey et al. in Comprehensive Organic Functional Group Transformations, 1^{st} Ed., Elsevier Science Ltd., Oxford, 1995, Vol. 5, pp. 257-308 and R. C. Larock in Comprehensive Organic Transformations, 2^{nd} Ed., Wiley-VCH, New York, Weinheim, 1999, pp. 1929-1994). The preparation of 4-hydroxy-2-oxo-1,2-dihydropyridine-3-carboxamides from 4-hydroxy-2-oxo-1,2-dihydropyridine-3-carboxylic esters is described, for example, in EP-A 0 693 477. Moreover, it is known that the reaction of carboxylic esters with amines can be accelerated by reaction auxiliaries such as trimethylaluminium, sodium cyanide or 2-hydroxypyridine (c.f. G. Benz in Comprehensive Organic Synthesis, loc. cit.).

The practice of process (e) by using 2-pyridones of the formula (I-f) in which X^{b} represents —C(═O)OR^{5b}

Step (e) of the process according to the invention is particularly advantageously carried out in the presence of 2-hydroxypyridine (c.f. J. Chem. Soc. (C), 1969, 89-91). Here, it is advantageous to carry out the reaction under superatmospheric pressure and, if appropriate, using microwave irradiation.

Diluents are advantageously employed in such an amount that the reaction mixture remains readily stirrable during the entire process.

Suitable diluents for carrying out step (e) of the process according to the invention are all inert organic solvents. These preferably include aliphatic, alicyclic or aromatic hydrocarbons, such as, for example, petroleum ether, pentane, hexane, heptane, octane, nonane and industrial hydrocarbons; for example white spirits with components having boiling points in the range of, for example, from 40° C. to 250° C., cymene, benzine fractions within a boiling point interval of from 70° C. to 190° C., cyclohexane, methylcyclohexane, benzene, toluene, xylene or decalin; halogenated hydrocarbons, such as, for example, chlorobenzene, dichlorobenzene, trichlorobenzene, chlorotoluene, dichloromethane, chloroform, carbon tetrachloride, 1,2-dichloroethane, trichloroethane, tetrachloroethane or tetrachloroethylene; ethers, such as diethyl ether, diisopropyl ether, methyl t-butyl ether, methyl t-amyl ether, dioxane, tetrahydrofuran, 1,2-dimethoxyethane, 1,2-diethoxyethane or anisol; nitro hydrocarbons, such as nitromethane, nitroethane, nitropropane, nitrobenzene, chloronitrobenzene, o-nitrotoluene; nitriles, such as acetonitrile, propionitrile, n- or i-butyronitrile or benzonitrile, m-chlorobenzonitrile; amides, such as N,N-dimethylformamide, N,N-dimethylacetamide, N-methylformanilide, N-methylpyrrolidone or hexamethylphosphoric triamide, and also dimethyl sulphoxide. It is also possible to use mixtures of the solvents and diluents mentioned. Preference is given to using toluene or chlorobenzene.

When carrying out step (e) of the process according to the invention, the reaction temperatures can be varied within a relatively wide range. In general, the process is carried out at temperatures of from −50° C. to +200° C., preferably at temperatures of from 0° C. to 150° C., particularly preferably at from 100° C. to 150° C.

For carrying out step (e) of the process according to the invention, any commercially available microwave apparatus suitable for this reaction (for example ETHOS 1600 from MLS GmbH, Leutkirch, Germany) can be used, if appropriate.

For carrying out step (e) of the process according to the invention for preparing the compounds of the formula (I-g), generally from 0.5 to 5 mol, preferably from 1 to 2 mol, of amine of the formula (VIII) are employed per mole of the 2-pyridone of the formula (I-f) [X^{b}═—C(═O)OR^{5b}]. The reaction time is from 10 minutes to 48 hours. Work-up is carried out by customary methods.

The practice of process (e) by using 2-pyridones of the formula (I-f) in which X^{b} represents —C(═O)OH

If appropriate, the process (e) according to the invention is carried out in the presence of a suitable acid acceptor. Suitable acid acceptors are all customary inorganic or organic bases. These preferably include alkaline earth metal or alkali metal hydrides, hydroxides, amides, alkoxides, acetates, carbonates or bicarbonates, such as, for example, sodium hydride, sodium amide, lithium diisopropylamide, sodium methoxide, sodium ethoxide, potassium tert-butoxide, sodium hydroxide, potassium hydroxide, sodium acetate, sodium carbonate, potassium carbonate, potassium bicarbonate, sodium bicarbonate or ammonium carbonate, and also tertiary amines, such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU).

If appropriate, the process (e) according to the invention is carried out in the presence of a suitable condensing agent. Suitable condensing agents are all condensing agents customarily used for such amidation reactions. Examples which may be mentioned are acid halide formers, such as phosgene, phosphorus tribromide, phosphorus trichloride, phosphorus pentachloride, phosphorus oxychloride, oxalyl chloride or thionyl chloride; anhydride formers, such as ethyl chloroformate, methyl chloroformate, isopropyl chloroformate, isobutyl chloroformate or methanesulphonyl chloride; carbodiimides, such as N,N′-dicyclohexylcarbodiimide (DCC), N-[3-(dimethylamino)propyl]-N′-ethylcarbodiimide hydrochloride, N,N′-di-sec-butylcarbodiimide, N,N′-diisopropylcarbodiimide, 1-(3-(dimethylamino)propyl)-3-ethylcarbodiimide methiodide, or other customary condensing agents, such as phorphorus pentoxide, polyphosphoric acid, N,N′-carbonyldiimidazol, 2-ethoxy-N-ethoxycarbonyl-1,2-dihydroquinoline (EEDQ), triphenylphosphine/carbon tetrachloride, bromotripyrrolidinophosphonium hexafluorophosphate, 2-bromo-3-ethyl-4-methylthiazolium tetrafluoroborate, N,N-bis[2-oxo-3-oxazolidinyl]phosphorodiamidine chloride, chlorotripyrrolidinophosphonium hexafluorophosphate, O-(1H-benzotriazol-1-yloxy)tris(dimethylamino)phosphonium hexafluorophosphate, N,N,N′,N′-bis(tetramethylene)chlorouronium hexafluoroborate, O-(1H-benzotriazol-1-yl)-N,N,N′,N′-tetramethyluronium hexafluorophosphate, O-(1H-benzotriazol-1-yl)-N,N,N′,N′-bis(tetramethylene)uronium hexafluorophosphate, O-(1H-benzotriazol-1-yl)-N,N,N′,N′-bis(tetramethylene)uronium tetrafluoroborate, O-(7-azabenzotriazol-1-yl)-N,N,N,N-tetramethyluronium hexafluorophosphate and 1-hydroxybenzotriazole. These reagents can be used separately or in combination.

Suitable diluents for carrying out the process (e) according to the invention are all inert organic solvents. These preferably include aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, tetrachloromethane, dichloroethane or trichloroethane; ethers, such as diethyl ether, diisopropyl ether, methyl tert-butyl ether, methyl tert-amyl ether, dioxane, tetrahydrofuran, 1,2-dimethoxyethane, 1,2-diethoxyethane or anisol, or amides, such as N,N-dimethylformamide, N,N-dimethylacetamide, N-methylformanilide, N-methylpyrrolidone or hexamethylphosphoric triamide.

When carrying out the process (e) according to the invention, the reaction temperatures can be varied within a relatively wide range. In general, the process is carried out at temperatures of from 0° C. to 150° C., preferably at temperatures of from 0° C. to 110° C., particularly preferably at from 0° C. to 40° C., very particularly preferably at room temperature.

For carrying out the process (e) according to the invention for preparing the compounds of the formula (I-g), in general from 0.2 to 5 mol, preferably from 0.5 to 2 mol, of amine of the formula (VIII), from 1 to 2 mol of the acid acceptor and from 1 to 2 mol of the coupling agent are employed per mole of the 2-pyridone of the formula (I-f) [X^{b}═—C(═O)OH]. Work-up is carried out by customary methods.

Process (3)

Using, for example, methyl 4-hydroxy-5,6-dimethoxy-1-(4-methoxybenzyl)-2-oxo-1,2-dihydropyridine-3-carboxylic acid as starting material, the course of the process (3) according to the invention can be illustrated by the following reaction equation:

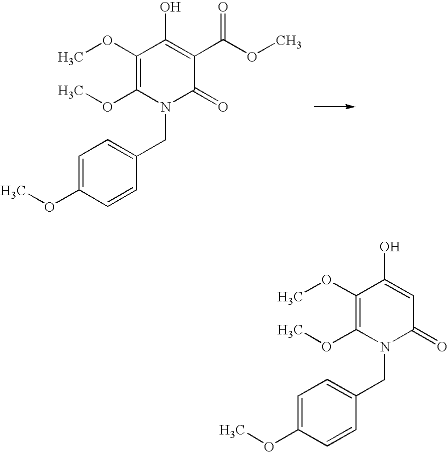

Step (f)

The formula (I-e) provides a general definition of the 2-pyridones required as starting materials for carrying out step (f) of the process according to the invention. In this formula, R¹, R² and R³ preferably, particularly preferably, very particularly preferably and especially preferably have those meanings which have already been given in connection with the description of the compounds of the formulae (I) and (II) which can be prepared according to the invention as being preferred, particularly preferred, etc., for these radicals.

The 2-pyridones of the formula (I-e) are a subset of the 2-pyridones of the formula (I). As far as they are comprised by the formula (I-k), they are novel. They can be prepared by process (1) according to the invention.

In general, it is advantageous to carry out step (f) of the process according to the invention in the presence of diluents and in the presence of basic reaction auxiliaries.

Diluents are advantageously employed in such an amount that the reaction mixture remains readily stirrable during the entire process.

Suitable diluents for carrying out step (f) of the process according to the invention are all inert organic solvents. These preferably include ethers, such as diethyl ether, diisopropyl ether, methyl t-butyl ether, methyl t-amyl ether, dioxane, tetrahydrofuran, 1,2-dimethoxyethane, 1,2-diethoxyethane or anisol; ketones, such as acetone, butanone, methyl isobutyl ketone or cyclohexanone; nitriles, such as acetonitrile, propionitrile, n- or i-butyronitrile or benzonitrile, m-chlorobenzonitrile; alcohols, such as methanol, ethanol, n- or i-propanol, n-, i-, sec- or tert-butanol, ethanediol, propane-1,2-diol, ethoxyethanol, methoxyethanol, amides, such as N,N-dimethylformamide, N,N-dimethylacetamide, N-methylformanilide, N-methylpyrrolidone or hexamethylphosphoric triamide, and also dimethyl sulphoxide or water. It is also possible to use mixtures of the solvents and diluents mentioned.

Preferred diluents for carrying out step (f) of the process according to the invention are ethers, such as diethyl ether, diisopropyl ether, methyl t-butyl ether, methyl t-amyl ether, dioxane, tetrahydrofuran, 1,2-dimethoxyethane, 1,2-diethoxyethane or anisol; ketones, such as acetone, butanone, methyl isobutyl ketone or cyclohexanone; alcohols, such as methanol, ethanol, n- or i-propanol, n-, i-, sec- or tert-butanol, ethanediol, propane-1,2-diol, ethoxyethanol, methoxyethanol, mixtures thereof with water and also pure water.

Suitable for use as basic reaction auxiliaries are, for example, alkali metal hydroxides, such as sodium hydroxide or potassium hydroxide.

When carrying out step (f) of the process according to the invention, the reaction temperatures can be varied within a relatively wide range. In general, the process is carried out at temperatures of from −50° C. to +150° C., preferably at temperatures of from 0° C. to +150° C., particularly preferably at from +30° C. to +100° C.

For carrying out step (f) of the process according to the invention, any commercially available microwave apparatus suitable for this reaction (for example ETHOS 1600 from MLS GmbH, Leutkirch, Germany) can be used, if appropriate.

For carrying out step (f) of the process according to the invention for preparing 2-pyridones of the formula (I-h), in general from 0.5 to 20 mol, preferably from 1 to 5 mol, of base are employed per mole of the 2-pyridone of the formula (I-e). The reaction time is from 15 minutes to 48 hours. The reaction is preferably carried out under an atmosphere of protective gas such as nitrogen or argon. Work-up is carried out by customary methods.

Process (4)

Using, for example, 4-hydroxy-5,6-dimethoxy-3-(5-methoxy-2-methylpentanoyl)pyridin-2(1H)-one as starting material and acetic anhydride as acylating agent, the course of the process (4) according to the invention can be illustrated by the following reaction equation:

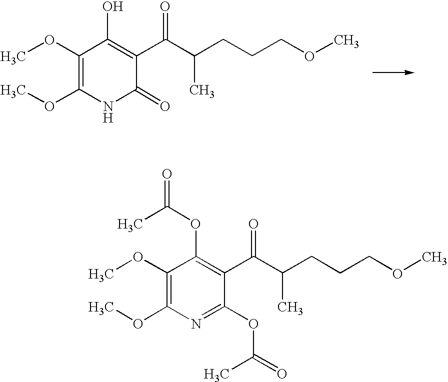

Step (g)

The formula (I-i) provides a general definition of the 2-pyridones required as starting materials for carrying out step (g) of the process according to the invention. In this formula R^{2a} preferably represents methyl. R^{2a} also preferably represents ethyl, particularly preferably methyl.

The 2-pyridones of the formula (I-i) are a subset of the 2-pyridones of the formula (I). As far as they are comprised by the formula (I-k) they are novel. They can be prepared by processes (1), (2) and (3) according to the invention.

The preferred acylating agents for carrying out step (g) of the process according to the invention are acetic anhydride and acetyl chloride. Particular preference is given to using acetic anhydride.

In general, it is advantageous to carry out step (g) of the process according to the invention in the presence of diluents, if appropriate, and in the presence of reaction auxiliaries, if appropriate.

Diluents are advantageously employed in such an amount that the reaction mixture remains readily stirrable during the entire process.

Suitable diluents for carrying out an acylation in step (g) of the process according to the invention are all inert organic solvents. These preferably include the following solvents: aliphatic, alicyclic or aromatic hydrocarbons, such as, for example, petroleum ether, pentane, hexane, heptane, octane, nonane and industrial hydrocarbons; for example white spirits with components having boiling points in the range of, for example, from 40° C. to 250° C., cymene, benzine fractions within a boiling point interval of from 70° C. to 190° C., cyclohexane, methylcyclohexane, benzene, toluene, xylene or decalin; halogenated hydrocarbons, such as, for example, chlorobenzene, dichlorobenzene, trichlorobenzene, chlorotoluene, dichloromethane, chloroform, carbon tetrachloride, 1,2-dichloroethane, trichloroethane, tetrachloroethane or tetrachloroethylene; ethers, such as diethyl ether, diisopropyl ether, methyl t-butyl ether, methyl t-amyl ether, dioxane, tetrahydrofuran, 1,2-dimethoxyethane, 1,2-diethoxyethane or anisol; esters, such as methyl acetate or ethyl acetate; ketones, such as acetone, butanone, methyl isobutyl ketone or cyclohexanone; nitriles, such as acetonitrile, propionitrile, n- or i-butyronitrile or benzonitrile, m-chlorobenzonitrile. It is also possible to use mixtures of the solvents and diluents mentioned. Particular preference is given to using toluene.

Suitable basic reaction auxiliaries for an acylation in step (g) of the process according to the invention are tertiary amines, such as trimethylamine, triethylamine, tributylamine, N,N-dimethylaniline, N,N-dimethylbenzylamine, pyridine, N-methylpiperidine, N-methylmorpholine, N,N-di-methylaminopyridine, diazabicyclooctane (DABCO), diazabicyclononene (DBN) or diazabicycloundecene (DBU), and also proton sponge (1,8-bisdimethylaminonaphthalene). Particular preference is given to using pyridine.

When carrying out an acylation in step (g) of the process according to the invention, the reaction temperatures can be varied within a relatively wide range. In general, the process is carried out at temperatures of from −78° C. to +150° C., preferably at temperatures of from 0° C. to +150° C., very particularly preferably at from +30° C. to +150° C.

For carrying out an acylation in step (g) of the process according to the invention for preparing 2-pyridinols of the formula (II), in general from 0.5 to 10 mol, preferably from 1 to 5 mol, of an acylating agent (for example acetic anhydride in pyridine as solvent) are used per mole of the 2-pyridone of the formula (I-i). The reaction time is from 1 to 48 hours. The reaction is preferably carried out under an atmosphere of protective gas such as nitrogen or argon. Work-up is carried out by customary methods.

The present invention furthermore relates to a composition for controlling unwanted microorganisms, comprising at least one of the active compounds according to the invention. These preferably take the form of fungicidal compositions which comprise agriculturally useable adjuvants, solvents, carriers, surfactants or extenders.

Moreover, the invention relates to a method for controlling unwanted microorganisms, characterized in that the active compounds according to the invention are applied to the phytopathogenic fungi and/or their habitat.

According to the invention, carrier is to be understood as meaning a natural or synthetic, organic or inorganic substance which is mixed or combined with the active compounds for better applicability, in particular for application to plants or plant parts or seeds. The carrier, which may be solid or liquid, is generally inert and should be suitable for use in agriculture.

Suitable solid or liquid carriers are: for example ammonium salts and ground natural minerals, such as kaolins, clays, talc, chalk, quartz, attapulgite, montmorillonite or diatomaceous earth, and ground synthetic minerals, such as finely divided silica, alumina and natural or synthetic silicates, resins, waxes, solid fertilizers, water, alcohols, especially butanol, organic solvents, mineral and vegetable oils and derivatives of these. Mixtures of such carriers may also be used. Suitable carriers for granules are: for example crushed and fractionated natural minerals, such as calcite, marble, pumice, sepiolite, dolomite, and also synthetic granules of inorganic and organic meals and also granules of organic material, such as sawdust, coconut shells, maize cobs and tobacco stalks.

Suitable liquefied gaseous extenders or carriers are liquids which are gaseous at ambient temperature and under atmospheric pressure, for example aerosol propellants, such as halocarbons, and also butane, propane, nitrogen and carbon dioxide.

Tackifiers, such as carboxymethylcellulose and natural and synthetic polymers in the form of powders, granules or latices, such as gum arabic, polyvinyl alcohol, polyvinyl acetate, or else natural phospholipids, such as cephalins and lecithins and synthetic phospholipids can be used in the formulations. Other possible additives are mineral and vegetable oils.

If the extender used is water, it is also possible, for example, to use organic solvents as auxiliary solvents. Suitable liquid solvents are essentially: aromatic compounds, such as xylene, toluene or alkylnaphthalenes, chlorinated aromatic compounds or chlorinated aliphatic hydrocarbons, such as chlorobenzenes, chloroethylenes or dichloromethane, aliphatic hydrocarbons, such as cyclohexane or paraffins, for example mineral oil fractions, mineral and vegetable oils, alcohols, such as butanol or glycol, and also ethers and esters thereof, ketones, such as acetone, methyl ethyl ketone, methyl isobutyl ketone or cyclohexanone, strongly polar solvents, such as dimethylformamide and dimethyl sulphoxide, and also water.

The compositions according to the invention may comprise additional further components, such as, for example, surfactants. Suitable surfactants are emulsifiers and/or foam formers, dispersants or wetting agents having ionic or nonionic properties, or mixtures of these surfactants. Examples of these are salts of polyacrylic acid, salts of lignosulphonic acid, salts of phenolsulphonic acid or naphthalenesulphonic acid, polycondensates of ethylene oxide with fatty alcohols or with fatty acids or with fatty amines, substituted phenols (preferably alkylphenols or arylphenols), salts of sulphosuccinic esters, taurine derivatives (preferably alkyl taurates), phosphoric esters of polyethoxylated alcohols or phenols, fatty esters of polyols, and derivatives of the compounds containing sulphates, sulphonates and phosphates, for example, alkylaryl polyglycol ethers, alkylsulphonates, alkyl sulphates, arylsulphonates, protein hydrolysates, ligno-sulphite waste liquors and methylcellulose. The presence of a surfactant is required if one of the active compounds and/or one of the inert carriers is insoluble in water and when the application takes place in water. The proportion of surfactants is between 5 and 40 per cent by weight of the composition according to the invention.

It is possible to use colorants such as inorganic pigments, for example iron oxide, titanium oxide, Prussian blue, and organic dyes, such as alizarin dyes, azo dyes and metal phthalocyanine dyes, and trace nutrients, such as salts of iron, manganese, boron, copper, cobalt, molybdenum and zinc.

If appropriate, other additional components may also be present, for example protective colloids, binders, adhesives, thickeners, thixotropic substances, penetrants, stabilizers, sequestering agents, complex formers. In general, the active compounds can be combined with any solid or liquid additive customarily used for formulation purposes.

In general, the compositions and formulations according to the invention contain between 0.05 and 99% by weight, 0.01 and 98% by weight, preferably between 0.1 and 95% by weight, especially preferably between 0.5 and 90% by weight of active compound, very especially preferably between 10 and 70 per cent by weight.

The active compounds or compositions according to the invention can be used as such or, depending on their respective physical and/or chemical properties, in the form of their formulations or the use forms prepared therefrom, such as aerosols, capsule suspensions, cold-fogging concentrates, warm-fogging concentrates, encapsulated granules, fine granules, flowable concentrates for the treatment of seed, ready-to-use solutions, dustable powders, emulsifiable concentrates, oil-in-water emulsions, water-in-oil emulsions, macrogranules, microgranules, oil-dispersible powders, oil-miscible flowable concentrates, oil-miscible liquids, foams, pastes, pesticide coated seed, suspension concentrates, suspoemulsion concentrates, soluble concentrates, suspensions, wettable powders, soluble powders, dusts and granules, water-soluble granules or tablets, water-soluble powders for the treatment of seed, wettable powders, natural products and synthetic substances impregnated with active compound, and also microencapsulations in polymeric substances and in coating materials for seed, and also ULV cold-fogging and warm-fogging formulations.

The formulations mentioned can be prepared in a manner known per se, for example by mixing the active compounds with at least one customary extender, solvent or diluent, emulsifier, dispersant and/or binder or fixing agent, wetting agent, water repellant, if appropriate siccatives and UV stabilizers and, if appropriate, dyes and pigments, defoamers, preservatives, secondary thickeners, adhesives, gibberellins and also further processing auxiliaries.

The compositions according to the invention do not only comprise ready-to-use compositions which can be applied with suitable apparatus to the plant or the seed, but also commercial concentrates which have to be diluted with water prior to use.

The active compounds according to the invention, per se or in their (commercially available) formulations and in the use forms prepared from these formulations, may be present in a mixture with other (known) active compounds such as insecticides, attractants, sterilants, bactericides, acaricides, nematicides, fungicides, growth regulators, herbicides, fertilizers, safeners or semiochemicals.

In many cases, synergistic effects are obtained, i.e. the activity of the mixture is greater than the activity of the individual components.

Suitable mixing partners are, for example, the following compounds:

Fungicides:

(1) Nucleic acid synthesis inhibitors, such as, for example, benalaxyl, benalaxyl-M, bupirimate, clozylacon, dimethirimol, ethirimol, furalaxyl, hymexazol, metalaxyl, metalaxyl-M, ofurace, oxadixyl and oxolic acid.

(2) Mitosis and cell division inhibitors, such as, for example, benomyl, carbendazim, chlorofenazole, diethofencarb, ethaboxam, fuberidazole, pencycuron, thiabendazole, thiophanate, thiophanate-, methyl and zoxamid.

(3) Respiration inhibitors (inhibitors of the respiratory chain), such as, for example, diflumetorim as inhibitor which acts on complex I of the respiratory chain; bixafen, boscalid, carboxin, fenfuram, flutolanil, fluopyram, furametpyr, furmecyclox, isopyrazam (9R component), isopyrazam (9S component), mepronil, oxycarboxin, penthiopyrad, thifluzamid as inhibitors which act on complex II of the respiratory chain; amisulbrom, azoxystrobin, cyazofamid, dimoxystrobin, enestroburin, famoxadone, fenamidone, fluoxastrobin, kresoxim-methyl, metominostrobin, orysastrobin, picoxystrobin, pyraclostrobin, pyribencarb, trifloxystrobin as inhibitors which act on complex III of the respiratory chain.

(4) Decouplers, such as, for example, binapacryl, dinocap, fluazinam and meptyldinocap.

(5) ATP production inhibitors, such as, for example, fentin acetate, fentin chloride, fentin hydroxide and silthiofam.

(6) Amino acid and protein biosynthesis inhibitors, such as, for example, andoprim, blasticidin-S, cyprodinil, kasugamycin, kasugamycin hydrochloride hydrate, mepanipyrim and pyrimethanil.

(7) Signal transduction inhibitors, such as, for example, fenpiclonil, fludioxonil and quinoxyfen.

(8) Lipid and membrane synthesis inhibitors, such as, for example, biphenyl, chlozolinate, edifenphos, etridiazole, iodocarb, iprobenfos, iprodione, isoprothiolane, procymidone, propamocarb, propamocarb hydrochloride, pyrazophos, tolclofos-methyl and vinclozolin.

(9) Ergosterol biosynthesis inhibitors, such as, for example, aldimorph, azaconazole, bitertanol, bromuconazole, cyproconazole, diclobutrazole, difenoconazole, diniconazole, diniconazole-M, dodemorph, dodemorph acetate, epoxiconazole, etaconazole, fenarimol, fenbuconazole, fenhexamid, fenpropidin, fenpropimorph, fluquinconazole, flurprimidol, flusilazole, flutriafol, furconazole, furconazole-cis, hexaconazole, imazalil, imazalil sulphate, imibenconazole, ipconazole, metconazole, myclobutanil, naftifine, nuarimol, oxpoconazole, paclobutrazole, pefurazoate, penconazole, piperalin, prochloraz, propiconazole, prothioconazole, pyributicarb, pyrifenox, quinconazole, simeconazole, spiroxamine, tebuconazole, terbinafine, tetraconazole, triadimefon, triadimenol, tridemorph, triflumizole, triforine, triticonazole, uniconazole, viniconazole and voriconazole.

(10) Cell wall synthesis inhibitors, such as, for example, benthiavalicarb, dimethomorph, flumorph, iprovalicarb, mandipropamid, polyoxins, polyoxorim, prothiocarb, validamycin A and valiphenal.

(11) Melanine biosynthesis inhibitors, such as, for example, carpropamid, diclocymet, fenoxanil, fthalide, pyroquilon and tricyclazole.

(12) Resistance inductors, such as, for example, acibenzolar-S-methyl, probenazole and tiadinil

(13) Compounds with multi-site activity, such as, for example, Bordeaux mixture, captafol, captan, chlorothalonil, copper naphthenate, copper oxide, copper oxychloride, copper preparations such as, for example, copper hydroxide, copper sulphate, dichlofluanid, dithianon, dodine and its free base, ferbam, fluorofolpet, folpet, guazatine, guazatine acetate, iminoctadine, iminoctadin albesilate, iminoctadin triacetate, man copper, man cozeb, maneb, metiram, metiram zinc, oxine-coppper, propamidine, propineb, sulphur and sulphur preparations such as, for example, calcium polysulphide, thiram, tolylfluanid, zineb and ziram.

(14) Further compounds, such as, for example, 2,3-dibutyl-6-chlorothieno[2,3-d]pyrimidin-4(3H)-one, ethyl(2Z)-3-amino-2-cyano-3-phenylprop-2-enoate, N-[2-(1,3-dimethylbutyl)phenyl]-5-fluoro-1,3-dimethyl-1H-pyrazole-4-carboxamide, N-{2-[1,1′-bi(cyclopropyl)-2-yl]phenyl}-3-(difluoromethyl)-1-methyl-1H-pyrazole-4-carboxamide, 3-(difluoromethyl)-1-methyl-N-(3′,4′,5′-trifluorobiphenyl-2-yl)-1H-pyrazole-4-carboxamide, 3-(difluoromethyl)-N-[4-fluoro-2-(1,1,2,3,3,3-hexafluoropropoxy)phenyl]-1-methyl-1H-pyrazole-4-carboxamide, (2E)-2-(2-{[6-(3-chloro-2-methylphenoxy)-5-fluoropyrimidin-4-yl]oxy}phenyl)-2-(methoxyimino)-N-methylethanamide, (2E)-2-{2-[({[(2E,3E)-4-(2,6-dichlorophenyl)but-3-en-2-ylidene]amino}oxy)methyl]phenyl}-2-(methoxyimino)-N-methylethanamide, 2-chloro-N-(1,1,3-trimethyl-2,3-dihydro-1H-inden-4-yl)pyridin-3-carboxamide, N-(3-ethyl-3,5,5-trimethylcyclohexyl)-3-(formylamino)-2-hydroxybenzamide, 5-methoxy-2-methyl-4-(2-{[({(1E)-1-[3-(trifluoromethyl)phenyl]ethylidene}amino)oxy]methyl}phenyl)-2,4-dihydro-3H-1,2,4-triazol-3-one, (2E)-2-(methoxyimino)-N-methyl-2-(2-{[({(1E)-1-[3-(trifluoromethyl)phenyl]ethylidene}amino)oxy]methyl}phenyl)ethaneamide, (2E)-2-(methoxyimino)-N-methyl-2-{2-[(E)-({1-[3-(trifluoromethyl)phenyl]ethoxy}imino)methyl] phenyl}ethaneamide, (2E)-2-{2-[({[(1E)-1-(3-{[(E)-1-fluoro-2-phenylethenyl]oxy}phenyl)ethylidene]amino}oxy)methyl]phenyl}-2-(methoxyimino)-N-methylethaneamide, 1-(4-chlorophenyl)-2-(1H-1,2,4-triazol-1-yl)cycloheptanol, methyl 1-(2,2-dimethyl-2,3-dihydro-1H-inden-1-yl)-1H-imidazole-5-carboxylate, N-ethyl-N-methyl-N′-{2-methyl-5-(trifluoromethyl)-4-[3-(trimethylsilyl)propoxy]phenyl}imidoformamide, N′-{5-(difluoromethyl)-2-methyl-4-[3-(trimethylsilyl)propoxy]phenyl}-N-ethyl-N-methylimidoformamide, O-{1-[(4-methoxyphenoxy)methyl]-2,2-dimethylpropyl}1H-imidazole-1-carbothioate, N-[2-(4-{[3-(4-chlorophenyl)prop-2-yn-1-yl]oxy}-3-methoxyphenyl)ethyl]-N²-(methylsulphonyl)valinamide, 5-chloro-7-(4-methylpiperidin-1-yl)-6-(2,4,6-trifluorophenyl)[1,2,4]triazolo[1,5-a]pyrimidine, 5-amino-1,3,4-thiadiazole-2-thiol, propamocarb-fosetyl, 1-[(4-methoxyphenoxylmethyl]-2,2-dimethylpropyl 1H-imidazole-1-carboxylate, 1-methyl-N-[2-(1,1,2,2-tetrafluoroethoxy)phenyl]-3-(trifluoromethyl)-1H-pyrazole-4-carboxamide, 2,3,5,6-tetrachloro-4-(methylsulphonyl)pyridine, 2-butoxy-6-iodo-3-propyl-4H-chromen-4-one, 2-phenylphenol and its salts, 3-(difluoromethyl)-1-methyl-N-[2-(1,1,2,2-tetrafluoroethoxy)phenyl]-1H-pyrazole-4-carboxamide, 3,4,5-trichloropyridine-2,6-dicarbonitrile, 3-[5-(4-chlorophenyl)-2,3-dimethylisoxazolidin-3-yl]pyridine, 3-chloro-5-(4-chlorophenyl)-4-(2,6-difluorophenyl)-6-methylpyridazine, 4-(4-chlorophenyl)-5-(2,6-difluorophenyl)-3,6-dimethylpyridazine, 8-hydroxyquinoline, 8-hydroxyquinoline sulphate, 5-methyl-6-octyl-3,7-dihydro[1,2,4]triazolo[1,5-a]pyrimidine-7-amine, 5-ethyl-6-octyl-3,7-dihydro[1,2,4]triazolo[1,5-a]pyrimidine-7-amine, benthiazole, bethoxazin, capsimycin, carvone, chinomethionat, chloroneb, cufraneb, cyflufenamide, cymoxanil, cyprosulphamide, dazomet, debacarb, dichlorophen, diclomezine, dicloran, difenzoquat, difenzoquat methylsulphate, diphenylamine, ecomat, ferimzone, flumetover, fluopicolide, fluoromide, flusulphamide, flutianil, fosetyl-aluminium, fosetyl-calcium, fosetyl-sodium, hexachlorobenzene, irumamycin, isotianil, methasulphocarb, methyl(2E)-2-{2-[({cyclopropyl[(4-methoxyphenyl)imino]methyl}thio)methyl]phenyl}-3-methoxyacrylate, methyl isothiocyanate, metrafenone, (5-bromo-2-methoxy-4-methylpyridin-3-yl)(2,3,4-trimethoxy-6-methylphenyl)methanone, mildiomycin, tolnifanide, N-(4-chlorobenzyl)-3-[3-methoxy-4-(prop-2-yn-1-yloxy)phenyl]propanamide, N-[(4-chlorophenyl)(cyano)methyl]-3-[3-methoxy-4-(prop-2-yn-1-yloxy)phenyl]propanamide, N-[(5-bromo-3-chloropyridin-2-yl)methyl]-2,4-dichloropyridine-3-carboxamide, N-[1-(5-bromo-3-chloropyridin-2-yl)ethyl]-2,4-dichloropyridine-3-carboxamide, N-[1-(5-bromo-3-chloropyridin-2-yl)ethyl]-2-fluoro-4-iodopyridine-3-carboxamide, N-{(Z)-[(cyclopropylmethoxy)imino][6-(difluoromethoxy)-2,3-difluorophenyl]methyl}-2-phenylacetamide, N-{(E)-[(cyclopropylmethoxy)imino][6-(difluoromethoxy)-2,3-difluorophenyl]methyl}-2-phenylacetamide, natamycin, nickel dimethyldithiocarbamate, nitrothalisopropyl, octhilinone, oxamocarb, oxyfenthiin, pentachlorophenol and its salts, phenazine-1-carboxylic acid, phenothrin, phosphoric acid and its salts, propamocarb fosetylate, propanosine-sodium, proquinazid, pyrrolnitrin, quintozene, S-prop-2-en-1-yl 5-amino-2-(1-methylethyl)-4-(2-methylphenyl)-3-oxo-2,3-dihydro-1H-pyrazole-1-carbothioate, tecloftalam, tecnazene, triazoxide, trichlamide, 5-chloro-N′-phenyl-N′-prop-2-yn-1-ylthiophene-2-sulphonohydrazide and zarilamide.

Bactericides:

Bronopol, dichlorophen, nitrapyrin, nickel dimethyldithiocarbamate, kasugamycin, octhilinone, furan carboxylic acid, oxytetracycline, probenazole, streptomycin, tecloftalam, copper sulphate and other copper preparations.

Insecticides/Akaricides/Nematicides:

(1) Acetyl cholinesterase (AChE) inhibitors, such as, for example,

Carbamates, for example alanycarb, aldicarb, aldoxycarb, allyxycarb, aminocarb, bendiocarb, benfuracarb, bufencarb, butacarb, butocarboxim, butoxycarboxim, carbaryl, carbofuran, carbosulphan, cloethocarb, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, trimethacarb, XMC and xylylcarb; or

organophosphates, for example acephate, azamethiphos, azinphos(-methyl, -ethyl), bromophos-ethyl, bromfenvinfos(-methyl), butathiofos, cadusafos, carbophenothion, chlorethoxyfos, chlorfenvinphos, chlormephos, chlorpyrifos(-methyl/-ethyl), coumaphos, cyanofenphos, cyanophos, chlorfenvinphos, demeton-S-methyl, demeton-S-methylsulphon, dialifos, diazinon, dichlofenthion, dichlorvos/DDVP, dicrotophos, dimethoate, dimethylvinphos, dioxabenzofos, disulphoton, EPN, ethion, ethoprophos, etrimfos, famphur, fenamiphos, fenitrothion, fensulphothion, fenthion, flupyrazofos, fonofos, formothion, fosmethilan, fosthiazate, heptenophos, iodofenphos, iprobenfos, isazofos, isofenphos, isopropyl, O-salicylate, isoxathion, malathion, mecarbam, methacrifos, methamidophos, methidathion, mevinphos, monocrotophos, naled, omethoate, oxydemeton-methyl, parathion(-methyl/-ethyl), phenthoate, phorate, phosalone, phosmet, phosphamidon, phosphocarb, phoxim, pirimiphos(-methyl/-ethyl), profenofos, propaphos, propetamphos, prothiofos, prothoate, pyraclofos, pyridaphenthion, pyridathion, quinalphos, sebufos, sulphotep, sulprofos, tebupirimfos, temephos, terbufos, tetrachlorvinphos, thiometon, triazophos, triclorfon, vamidothion and imicyafos.

(2) GABA-gated chloride channel antagonists, such as, for example, organochlorines, for example camphechlor, chlordane, endosulphan, gamma-HCH, HCH, heptachlor, lindane and methoxychlor; or fiproles (phenylpyrazoles), for example acetoprole, ethiprole, fipronil, pyrafluprole, pyriprole and vaniliprole.

(3) Sodium channel modulators/voltage-dependent sodium channel blockers, such as, for example, pyrethroides, for example acrinathrin, allethrin (d-cis-trans, d-trans), beta-cyfluthrin, bifenthrin, bioallethrin, bioallethrin-S-cyclopentyl isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, deltamethrin, empenthrin (1R-isomer), esfenvalerate, etofenprox, fenfluthrin, fenpropathrin, fenpyrithrin, Fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R-trans isomer), prallethrin, profluthrin, protrifenbute, pyresmethrin, resmethrin, RU 15525, silafluofen, tau-fluvalinate, tefluthrin, terallethrin, tetramethrin(-1R-isomer), tralomethrin, transfluthrin, ZXI 8901, pyrethrin (pyrethrum), eflusilanat; DDT; or methoxychlor.

(4) Nicotinergic acetylcholine receptor agonists/antagonists, such as, for example, chloronicotinyls, for example acetamiprid, clothianidin, dinotefuran, imidacloprid, imidaclothiz, nitenpyram, nithiazine, thiacloprid, thiamethoxam, AKD-1022; nicotine, bensultap, cartap, thiosultap-sodium and thiocylam.

(5) Allosteric acetylcholine receptor modulators (agonists), such as, for example, spinosyns, for example spinosad and spinetoram.

(6) Chloride channel activators, such as, for example, mectines/Macrolides, for example abamectin, emamectin, emamectin-benzoate, ivermectin, lepimectin and milbemectin; or juvenile hormone analogues, for example hydroprene, kinoprene, methoprene, epofenonane, triprene, fenoxycarb, pyriproxifen and diofenolan.

(7) Active compounds with unknown or non-specific mechanisms of action, such as, for example, fumigants, for example methyl bromide, chloropicrin and sulphuryl fluoride; selective antifeedants, for example cryolite, pymetrozine, pyrifluquinazon and flonicamid; or mite growth inhibitors, for example clofentezine, hexythiazox, etoxazole.

(8) Oxidative phosphorylation inhibitors, ATP disruptors, such as, for example, diafenthiuron; organotin compounds, for example azocyclotin, cyhexatin and fenbutatin oxides; or propargite, tetradifon.

(9) Oxidative phoshorylation decouplers acting by interrupting the H proton gradient, such as, for example, chlorfenapyr, binapacyrl, dinobuton, dinocap and DNOC.

(10) Microbial disruptors of the insect gut membrane, such as, for example, Bacillus thuringiensis-strains.

(11) Chitin biosynthesis inhibitors, such as, for example, benzoylureas, for example bistrifluron, chlorfluazuron, diflubenzuron, fluazuron, flucycloxuron, flufenoxuron, hexaflumuron, lufenuron, novaluron, noviflumuron, penfluron, teflubenzuron or triflumuron.

(12) Buprofezin.

(13) Moulting disruptors, such as, for example, cyromazine.

(14) Ecdysone agonists/disruptors, such as, for example, diacylhydrazines, for example chromafenozide, halofenozide, methoxyfenozide, tebufenozide and fufenozide (JS 118); or azadirachtin.

(15) Octopaminergic agonists, such as, for example, amitraz.

(16) Site III electron transport inhibitors/site II electron transport inhibitors, such as, for example, hydramethylnon; acequinocyl; fluacrypyrim; or cyflumetofen and cyenopyrafen.

(17) Electron transport inhibitors, such as, for example, site I electron transport inhibitors, from the group of the METI-akarizides, for example fenazaquin, fenpyroximate, pyrimidifen, pyridaben, tebufenpyrad, tolfenpyrad, and rotenone; or voltage-dependent sodium channel blockers, for example indoxacarb and metaflumizone.

(18) Fatty acid biosynthesis inhibitors, such as, for example, tetronic acid derivatives, for example spirodiclofen and spiromesifen; or tetramic acid derivatives, for example spirotetramat.

(19) Neuronal inhibitors having an unknown mechanism of action, for example bifenazate.

(20) Ryanodin receptor effectors, such as, for example, diamides, for example flubendiamide, (R)-, (S)-3-chloro-N¹-{2-methyl-4-[1,2,2,2-tetrafluoro-1-(trifluoromethyl)ethyl]phenyl}-N²-(1-methyl-2-methyl-sulphonylethyl)phthalamide, chloroantraniliprole (Rynaxypyr), or cyantraniliprole (Cyazypyr).

(21) Further active compounds with an unknown mechanism of action, such as, for example, amidoflumet, benclothiaz, benzoximate, bromopropylate, buprofezin, chinomethionat, chlorodimeform, chlorobenzilate, clothiazoben, cycloprene, dicofol, dicyclanil, fenoxacrim, fentrifanil, flubenzimine, flufenerim, flutenzin, gossyplure, japonilure, metoxadiazone, petroleum, potassium oleate, pyridalyl, sulphluramide, tetrasul, triarathene, or verbutin; or the known active compounds below, 4-{[(6-bromopyrid-3-yl)methyl](2-fluoroethyl)amino}furan-2(5H)-one, 4-{[(6-fluoropyrid-3-yl)methyl](2,2-difluoroethyl)amino}furan-2(5H)-one, 4-{[(2-chloro-1,3-thiazol-5-yl)methyl](2-fluoroethyl)amino}furan-2(5H)-one, 4-{[(6-chloropyrid-3-yl)methyl](2-fluoroethyl)amino}furan-2(5H)-one, 4-{[(6-chloropyrid-3-yl)methyl](2,2-difluoroethyl)amino}furan-2(5H)-one (all known from WO 2007/115644), 4-{[(5,6-dichloropyrid-3-yl)methyl](2-fluoroethyl)amino}furan-2(5H)-one (known from WO 2007/115646), 4-{[(6-chloro-5-fluoropyrid-3-yl)methyl](methyl)amino}furan-2(5H)-one, 4-{[(6-chloro-5-fluoropyrid-3-yl)methyl](cyclopropyl)amino}furan-2(5H)-one (both known from WO 2007/115643), 4-{[(6-chloropyrid-3-yl)methyl](cyclopropyl)amino}furan-2(5H)-one, 4-{[(6-chloropyrid-3-yl)methyl](methyl)amino}furan-2(5H)-one (both known from EP-A 0 539 588), [(6-chloropyridin-3-yl)methyl](methyl)oxido-λ⁴-sulphanylidenecyanamide, [1-(6-chloropyridin-3-yl)ethyl](methyl)oxido-λ⁴-sulphanylidenecyanamide (both known from WO 2007/149134) and its diastereomers (A) and (B)

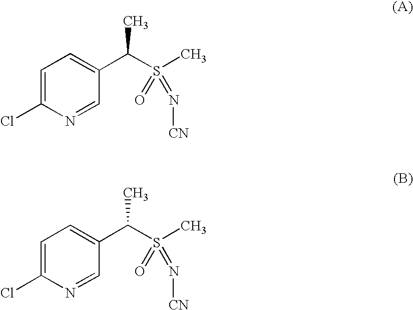

(also known from WO 2007/149134), [(6-trifluoromethylpyridin-3-yl)methyl](methyl)oxido-λ⁴-sulphanylidenecyanamide (known from WO 2007/095229), or [1-(6-trifluoromethylpyridin-3-yl)ethyl](methyl)oxido-λ⁴-sulphanylidenecyanamide (known from WO 2007/149134) and its diastereomers (C) and (D), namely sulphoxaflor (also known from WO 2007/149134)

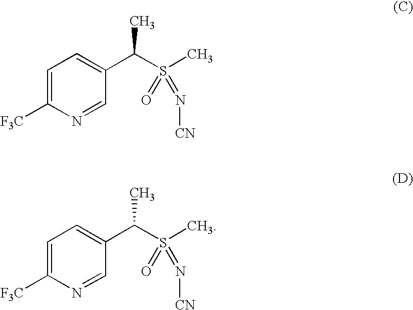

The treatment according to the invention of the plants and plant parts with the active compounds or compositions is carried out directly or by action on their surroundings, habitat or storage space using customary treatment methods, for example by dipping, spraying, atomizing, irrigating, evaporating, dusting, fogging, broadcasting, foaming, painting, spreading-on, drenching, drip irrigating and, in the case of propagation material, in particular in the case of seeds, furthermore by dry seed treatment, by wet seed treatment, by slurry treatment, by incrusting, by coating with one or more coats, etc. It is furthermore possible to apply the active compounds by the ultra-low-volume method, or to inject the active compound preparation, or the active compound itself, into the soil.

The invention furthermore comprises a method for the treatment of seed.

The invention furthermore relates to seed which has been treated in accordance with one of the methods described in the previous paragraph. The seeds according to the invention are used in methods for the protection of seed from undesirable microorganisms. Here, a seed treated with at least one active compound according to the invention is used.

The active compounds or compositions according to the invention are also suitable for treating seed. A large part of the damage to crop plants caused by harmful organisms is triggered by the infection of the seed during storage or after sowing as well as during and after germination of the plant. This phase is particularly critical since the roots and shoots of the growing plant are particularly sensitive, and even just small damage may result in the death of the plant. Accordingly, there is great interest in protecting the seed and the germinating plant by using appropriate compositions.

The control of phytopathogenic fungi by treating the seed of plants has been known for a long time and is the subject of continuous improvements. However, the treatment of seed entails a series of problems which cannot always be solved in a satisfactory manner Thus, it is desirable to develop methods for protecting the seed and the germinating plant which dispense with the additional application of plant protection compositions after sowing or after the emergence of the plants or which at least considerably reduce additional application. It is furthermore desirable to optimize the amount of active compound employed in such a way as to provide maximum protection for the seed and the germinating plant from attack by phytopathogenic fungi, but without damaging the plant itself by the active compound employed. In particular, methods for the treatment of seed should also take into consideration the intrinsic fungicidal properties of transgenic plants in order to achieve optimum protection of the seed and the germinating plant with a minimum of plant protection compositions being employed.

Accordingly, the present invention also relates to a method for protecting seed and germinating plants against attack by phytopathogenic fungi by treating the seed with a composition according to the invention. The invention also relates to the use of the compositions according to the invention for treating seed for protecting the seed and the germinating plant against phytopathogenic fungi. Furthermore, the invention relates to seed treated with a composition according to the invention for protection against phytopathogenic fungi.

The control of phytopathogenic fungi which damage plants post-emergence is carried out primarily by treating the soil and the above-ground parts of plants with plant protection compositions. Owing to the concerns regarding a possible impact of the plant protection compositions on the environment and the health of humans and animals, there are efforts to reduce the amount of active compounds applied.

One of the advantages of the present invention is that, because of the particular systemic properties of the active compounds or compositions according to the invention, treatment of the seed with these active compounds or compositions not only protects the seed itself, but also the resulting plants after emergence, from phytopathogenic fungi. In this manner, the immediate treatment of the crop at the time of sowing or shortly thereafter can be dispensed with.

It is also considered to be advantageous that the active compounds or compositions according to the invention can be used in particular also for transgenic seed where the plant growing from this seed is capable of expressing a protein which acts against pests. By treating such seed with the active compounds or compositions according to the invention, even by the expression of the, for example, insecticidal protein, certain pests may be controlled. Surprisingly, a further synergistic effect may be observed here, which additionally increases the effectiveness of the protection against attack by pests.

The compositions according to the invention are suitable for protecting seed of any plant variety employed in agriculture, in the greenhouse, in forests or in horticulture and viticulture. In particular, this takes the form of seed of cereals (such as wheat, barley, rye, triticale, sorghum/millet and oats), maize, cotton, soya beans, rice, potatoes, sunflower, bean, coffee, beet (for example sugar beet and fodder beet), peanut, oilseed rape, poppy, olive, coconut, cacao, sugar cane, tobacco, vegetables (such as tomato, cucumbers, onions and lettuce), turf and ornamentals (see also hereinbelow). Of particular importance is the treatment of the seed of cereals (such as wheat, barley, rye, triticale and oats), maize and rice.

As also described hereinbelow, the treatment of transgenic seed with the active compounds or compositions according to the invention is of particular importance. This refers to the seed of plants containing at least one heterologous gene which allows the expression of a polypeptide or protein having insecticidal properties. The heterologous gene in transgenic seed can originate, for example, from microorganisms of the species Bacillus, Rhizobium, Pseudomonas, Serratia, Trichoderma, Clavibacter, Glomus or Gliocladium. Preferably, this heterologous gene is from Bacillus sp., the gene product having activity against the European corn borer and/or the Western corn rootworm. Particularly preferably, the heterologous gene originates from Bacillus thuringiensis.

In the context of the present invention, the composition according to the invention is applied on its own or in a suitable formulation to the seed. Preferably, the seed is treated in a state in which it is sufficiently stable so that the treatment does not cause any damage. In general, treatment of the seed may take place at any point in time between harvesting and sowing. Usually, the seed used has been separated from the plant and freed from cobs, shells, stalks, coats, hairs or the flesh of the fruits. Thus, it is possible to use, for example, seed which has been harvested, cleaned and dried to a moisture content of less than 15% by weight. Alternatively, it is also possible to use seed which, after drying, has been treated, for example, with water and then dried again.

When treating the seed, care must generally be taken that the amount of the composition according to the invention applied to the seed and/or the amount of further additives is chosen in such a way that the germination of the seed is not adversely affected, or that the resulting plant is not damaged. This must be borne in mind in particular in the case of active compounds which may have phytotoxic effects at certain application rates.

The compositions according to the invention can be applied directly, that is to say without comprising further components and without having been diluted. In general, it is preferable to apply the compositions to the seed in the form of a suitable formulation. Suitable formulations and methods for the treatment of seed are known to the person skilled in the art and are described, for example, in the following documents: U.S. Pat. No. 4,272,417 A, U.S. Pat. No. 4,245,432 A, U.S. Pat. No. 4,808,430 A, U.S. Pat. No. 5,876,739 A, US 2003/0176428 A1, WO 2002/080675 A1, WO 2002/028186 A2.

The active compounds which can be used according to the invention can be converted into the customary seed-dressing product formulations such as solutions, emulsions, suspensions, powders, foams, slurries and other coating compositions for seed, and ULV formulations.

These formulations are prepared in the known manner by mixing the active compounds with customary additives such as, for example, customary extenders and also solvents or diluents, colorants, wetters, dispersants, emulsifiers, antifoams, preservatives, secondary thickeners, adhesives, gibberellins, and also water.

Colorants which may be present in the seed-dressing product formulations which can be used according to the invention are all colorants which are customary for such purposes. Both pigments, which are sparingly soluble in water, and dyes, which are soluble in water, may be used. Examples of colorants which may be mentioned are those known by the names Rhodamin B, C.I. Pigment Red 112 and C.I. Solvent Red 1.

Wetters which may be present in the seed-dressing product formulations which can be used according to the invention are all substances which are conventionally used for the formulation of agrochemical active compounds and for promoting wetting. Alkylnaphthalenesulphonates, such as diisopropyl- or diisobutylnaphthalenesulphonates, can preferably be used.

Suitable dispersants and/or emulsifiers which may be present in the seed-dressing product formulations which can be used in accordance with the invention are all non-ionic, anionic and cationic dispersants which are conventionally used for the formulation of agrochemical active compounds. Non-ionic or anionic dispersants or mixtures of non-ionic or anionic dispersants can preferably be used. Suitable non-ionic dispersants which may be mentioned are, in particular, ethylene oxide/propylene oxide block polymers, alkylphenol polyglycol ethers and tristryrylphenol polyglycol ethers, and their phosphated or sulphated derivatives. Suitable anionic dispersants are, in particular, lignosulphonates, polyacrylic acid salts and arylsulphonate/formaldehyde condensates.

Antifoams which may be present in the seed-dressing product formulations which can be used according to the invention are all foam-suppressing substances conventionally used for the formulation of agrochemical active compounds. Silicone antifoams and magnesium stearate can preferably be used.

Preservatives which may be present in the seed-dressing product formulations which can be used according to the invention are all substances which can be employed in agrochemical compositions for such purposes. Examples which may be mentioned are dichlorophene and benzyl alcohol hemiformal.

Secondary thickeners which may be present in the seed-dressing product formulations which can be used according to the invention are all substances which can be employed in agrochemical compositions for such purposes. Cellulose derivatives, acrylic acid derivatives, xanthan, modified clays and highly disperse silica are preferably suitable.

Adhesives which may be present in the seed-dressing product formulations which can be used according to the invention are all customary binders which can be employed in seed-dressing products. Polyvinylpyrrolidone, polyvinyl acetate, polyvinyl alcohol and tylose may be mentioned by preference.

Gibberellins which may be present in the seed-dressing product formulations which can be used according to the invention are preferably the gibberellins A1, A3 (=gibberellic acid), A4 and A7, with gibberellic acid being particularly preferably used. The gibberellins are known (cf. R. Wegler “Chemie der Pflanzenschutz- und Schadlingsbekampfungsmittel” [Chemistry of Plant Protectants and Pesticides], Vol. 2, Springer Verlag, 1970, pp. 401-412).

The seed-dressing product formulations which can be used in accordance with the invention can be employed either directly or after previous dilution with water for the treatment of a wide range of seeds, including the seed of transgenic plants. In this context, additional synergistic effects may also occur as a consequence of the interaction with the substances formed by expression.

Suitable apparatuses which can be employed for treating seed with the seed-dressing product formulations which can be used in accordance with the invention, or with the preparations prepared therefrom by addition of water, are all mixing apparatuses which can usually be employed for dressing seed. Specifically, a seed-dressing procedure is followed in which the seed is placed in a mixer, the amount of seed-dressing product formulation desired in each case is added, either as such or after previously diluting it with water, and the contents of the mixer are mixed until the formulation has been distributed uniformly on the seed. If appropriate, this is followed by a drying process.

The active compounds or compositions according to the invention have a potent microbicidal activity and can be employed for controlling unwanted microorganisms such as fungi and bacteria in plant protection and in the protection of materials.

Fungicides can be used in crop protection for controlling Plasmodiophoromycetes, Oomycetes, Chytridiomycetes, Zygomycetes, Ascomycetes, Basidiomycetes und Deuteromycetes.

Bactericides can be used in crop protection for controlling Pseudomonadaceae, Rhizobiaceae, Enterobacteriaceae, Corynebacteriaceae and Streptomycetaceae.

The fungicidal compositions according to the invention can be employed curatively or protectively for controlling phytopathogenic fungi. The invention therefore also relates to curative and protective methods of controlling phytopathogenic fungi by using the active compounds or compositions according to the invention, which are applied to the seed, the plant or plant parts, the fruits or the soil in which the plants grow.

The compositions according to the invention for controlling phytopathogenic fungi in plant protection comprise an effective, but nonphytotoxic amount of the active compounds according to the invention. “Effective, but nonphytotoxic amount” means such an amount of the composition according to the invention which suffices for sufficiently controlling or fully eradicating the fungal disease of the plant while simultaneously not entailing substantial phytotoxicity symptoms. In general, this application rate can vary within a substantial range. It depends on a plurality of factors, for example on the fungus to be controlled, the plant, the climatic conditions and the constituents of the compositions according to the invention.

The good plant tolerance of the active compounds at the concentrations required for controlling plant diseases permits the treatment of aerial plant parts, of vegetative propagation material and of seed, and of the soil.

All plants and plant parts can be treated in accordance with the invention. In the present context, plants are understood as meaning all plants and plant populations, such as desired and undesired wild plants or crop plants (including naturally occurring crop plants). Crop plants can be plants which can be obtained by traditional breeding and optimization methods or by biotechnological and recombinant methods, or combinations of these methods, including the transgenic plants and including the plant varieties capable or not of being protected by Plant Breeders' Rights. Plant parts are understood as meaning all aerial and subterranean parts and organs of the plants, such as shoot, leaf, flower and root, examples which may be mentioned being leaves, needles, stalks, stems, flowers, fruiting bodies, fruits and seeds, and also roots, tubers and rhizomes. The plant parts also include crop material and vegetative and generative propagation material, for example cuttings, tubers, rhizomes, slips and seeds.

The active compounds according to the invention are suitable for the protection of plants and plant organs, for increasing the yields, for improving the quality of the harvested crop, while being well tolerated by plants, having favourable toxicity to warm-blooded species and being environmentally friendly. They can preferably be employed as plant protection compositions. They are active against normally sensitive and resistant species and against all or individual developmental stages.

Plants which can be treated in accordance with the invention and which may be mentioned are the following: cotton, flax, grapevine, fruit, vegetables, such as Rosaceae sp. (for example pome fruits such as apples and pears, but also stone fruits such as apricots, cherries, almonds and peaches, and soft fruits such as strawberries), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for example banana plants and banana plantations), Rubiaceae sp. (for example coffee), Theaceae sp., Sterculiceae sp., Rutaceae sp. (for example lemons, oranges and grapefruit); Solanaceae sp. (for example tomatoes), Liliaceae sp., Asteraceae sp. (for example lettuce), Umbelliferae sp., Cruciferae sp., Chenopodiaceae sp., Cucurbitaceae sp. (for example cucumbers), Alliaceae sp. (for example leeks, onions), Papilionaceae sp. (for example peas); major crop plants such as Gramineae sp. (for example maize, turf, cereals such as wheat, rye, rice, barley, oats, sorghum, millet and triticale), Asteraceae sp. (for example sunflower), Brassicaceae sp. (for example white cabbage, red cabbage, broccoli, cauliflower, Brussels sprouts, pak choi, kohlrabi, small radishes, and also oilseed rape, mustard, horseradish and cress), Fabacae sp. (for example beans, peanuts), Papilionaceae sp. (for example soya beans), Solanaceae sp. (for example potatoes), Chenopodiaceae sp. (for example sugar beet, fodder beet, Swiss chard, beetroot); useful plants and ornamental plants in gardens and forests; and in each case genetically modified types of these plants.

As has already been mentioned above, all plants and their parts may be treated in accordance with the invention. In a preferred embodiment, plant species and plant varieties, and their parts, which grow wild or which are obtained by traditional biological breeding methods such as hybridization or protoplast fusion are treated. In a further preferred embodiment, transgenic plants and plant varieties which have been obtained by recombinant methods, if appropriate in combination with traditional methods (genetically modified organisms), and their parts are treated. The term “parts” or “parts of plants” or “plant parts” has been explained hereinabove. Plants of the plant varieties which are in each case commercially available or in use are especially preferably treated in accordance with the invention. Plant varieties are understood as meaning plants with novel traits which have been bred both by traditional breeding, by mutagenesis or by recombinant DNA techniques. They may take the form of varieties, races, biotypes and genotypes.

The method of treatment according to the invention can be used in the treatment of genetically modified organisms (GMOs), e.g. plants or seeds. Genetically modified plants (or transgenic plants) are plants in which a heterologous gene has been stably integrated into the genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which are present in the plant (using for example antisense technology, cosuppression technology or RNA interference-RNAi technology). A heterologous gene that is located in the genome is also called a transgene. A transgene that is defined by its particular location in the plant genome is called a transformation or transgenic event.

Depending on the plant species or plant varieties, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the active compounds and compositions which can be used according to the invention, better plant growth, increased tolerance to high or low temperatures, increased tolerance to drought or to water or soil salt content, increased flowering performance, easier harvesting, accelerated maturation, higher harvest yields, bigger fruits, larger plant height, greener leaf colour, earlier flowering, higher quality and/or a higher nutritional value of the harvested products, higher sugar concentration within the fruits, better storage stability and/or processability of the harvested products are possible, which exceed the effects which were actually to be expected.

At certain application rates, the active compound combinations according to the invention may also have a strengthening effect in plants. Accordingly, they are suitable for mobilizing the defence system of the plant against attack by unwanted phytopathogenic fungi and/or microorganisms and/or viruses. This may, if appropriate, be one of the reasons for the enhanced activity of the combinations according to the invention, for example against fungi. Plant-strengthening (resistance-inducing) substances are to be understood as meaning, in the present context, also those substances or combinations of substances which are capable of stimulating the defence system of plants in such a way that, when subsequently inoculated with unwanted phytopathogenic fungi, the treated plants display a substantial degree of resistance to these unwanted phytopathogenic fungi. Thus, the substances according to the invention can be employed for protecting plants against attack by the abovementioned pathogens within a certain period of time after the treatment. The period of time within which protection is effected generally extends from 1 to 10 days, preferably 1 to 7 days, after the treatment of the plants with the active compounds.

Plants and plant varieties which are preferably to be treated according to the invention include all plants which have genetic material which imparts particularly advantageous, useful traits to these plants (whether obtained by breeding and/or biotechnological means).

Plants and plant varieties which are also preferably to be treated according to the invention are resistant against one or more biotic stresses, i.e. said plants have a better defence against animal and microbial pests, such as against nematodes, insects, mites, phytopathogenic fungi, bacteria, viruses and/or viroids.

Plants and plant varieties which may also be treated according to the invention are those plants which are resistant to one or more abiotic stresses. Abiotic stress conditions may include, for example, drought, cold temperature exposure, heat exposure, osmotic stress, waterlogging, increased soil salinity, increased exposure to minerals, exposure to ozone, exposure to strong light, limited availability of nitrogen nutrients, limited availability of phosphorus nutrients or shade avoidance.

Plants and plant varieties which may also be treated according to the invention are those plants characterized by enhanced yield characteristics. Enhanced yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore be affected by improved plant architecture (under stress and non-stress conditions), including early flowering, flowering control for hybrid seed production, seedling vigour, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content, protein content, oil content and composition, nutritional value, reduction in anti-nutritional compounds, improved processability and better storage stability.

Plants that may be treated according to the invention are hybrid plants that already express the characteristics of heterosis, or hybrid vigour, which results in generally higher yield, vigour, health and resistance towards biotic and abiotic stress factors. Such plants are typically made by crossing an inbred male-sterile parent line (the female parent) with another inbred male-fertile parent line (the male parent). Hybrid seed is typically harvested from the male sterile plants and sold to growers. Male sterile plants can sometimes (e.g. in corn) be produced by detasseling (i.e. the mechanical removal of the male reproductive organs or male flowers) but, more typically, male sterility is the result of genetic determinants in the plant genome. In that case, and especially when seed is the desired product to be harvested from the hybrid plants, it is typically useful to ensure that male fertility in the hybrid plants, which contain the genetic determinants responsible for male sterility, is fully restored. This can be accomplished by ensuring that the male parents have appropriate fertility restorer genes which are capable of restoring the male fertility in hybrid plants that contain the genetic determinants responsible for male sterility. Genetic determinants for male sterility may be located in the cytoplasm. Examples of cytoplasmic male sterility (CMS) were for instance described in Brassica species. However, genetic determinants for male sterility can also be located in the nuclear genome. Male sterile plants can also be obtained by plant biotechnology methods such as genetic engineering. A particularly useful means of obtaining male sterile plants is described in WO 89/10396 in which, for example, a ribonuclease such as barnase is selectively expressed in the tapetum cells in the stamens. Fertility can then be restored by expression in the tapetum cells of a ribonuclease inhibitor such as barstar.

Plants or plant cultivars (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Herbicide-tolerant plants are for example glyphosate-tolerant plants, i.e. plants made tolerant to the herbicide glyphosate or salts thereof. For example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium, the CP4 gene of the bacterium Agrobacterium sp., the genes encoding a petunia EPSPS, a tomato EPSPS, or an Eleusine EPSPS. It can also be a mutated EPSPS. Glyphosate-tolerant plants can also be obtained by expressing a gene that encodes a glyphosate oxidoreductase enzyme. Glyphosate-tolerant plants can also be obtained by expressing a gene that encodes a glyphosate acetyltransferase enzyme. Glyphosate-tolerant plants can also be obtained by selecting plants containing naturally occurring mutations of the abovementioned genes.

Other herbicide-resistant plants are for example plants that are made tolerant to herbicides inhibiting the enzyme glutamine synthase, such as bialaphos, phosphinothricin or glufosinate. Such plants can be obtained by expressing an enzyme detoxifying the herbicide or a mutant glutamine synthase enzyme that is resistant to inhibition. One such efficient detoxifying enzyme is, for example, an enzyme encoding a phosphinothricin acetyltransferase (such as the bar or pat protein from Streptomyces species). Plants expressing an exogenous phosphinothricin acetyltransferase are described.

Further herbicide-tolerant plants are also plants that are made tolerant to the herbicides inhibiting the enzyme hydroxyphenylpyruvatedioxygenase (HPPD). Hydroxyphenylpyruvatedioxygenases are enzymes that catalyze the reaction in which para-hydroxyphenylpyruvate (HPP) is transformed into homogentisate. Plants tolerant to HPPD inhibitors can be transformed with a gene encoding a naturally occurring resistant HPPD enzyme, or a gene encoding a mutated HPPD enzyme. Tolerance to HPPD inhibitors can also be obtained by transforming plants with genes encoding certain enzymes enabling the formation of homogentisate despite the inhibition of the native HPPD enzyme by the HPPD inhibitor. Tolerance of plants to HPPD inhibitors can also be improved by transforming plants with a gene encoding an enzyme of prephenate dehydrogenase in addition to a gene encoding an HPPD-tolerant enzyme.

Still further herbicide-resistant plants are plants that are made tolerant to acetolactate synthase (ALS) inhibitors. Known ALS inhibitors include, for example, sulphonylurea, imidazolinone, triazolopyrimidines, pyrimidinyloxy(thio)benzoates, and/or sulphonylaminocarbonyltriazolinone herbicides. Different mutations in the ALS enzyme (also known as acetohydroxyacid synthase, AHAS) are known to confer tolerance to different herbicides and groups of herbicides. The production of sulphonylurea-tolerant plants and imidazolinone-tolerant plants has been described in the international publication WO 1996/033270. Further sulphonylurea- and imidazolinone-tolerant plants have also been described, for example in WO 2007/024782.

Other plants tolerant to imidazolinone and/or sulphonylurea can be obtained by induced mutagenesis, selection in cell cultures in the presence of the herbicide or mutation breeding.

Plants or plant varieties (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance.

An “insect-resistant transgenic plant”, as used herein, includes any plant containing at least one transgene comprising a coding sequence encoding:
1) an insecticidal crystal protein from Bacillus thuringiensis or an insecticidal portion thereof, such as the insecticidal crystal proteins listed online at:
   http://www.lifesci.sussex.ac.uk/Home/Neil_Crickmore/Bt/, or insecticidal portions thereof, e.g. proteins of the Cry protein classes Cry1Ab, Cry1Ac, Cry1F, Cry2Ab, Cry3Ae, or Cry3Bb or insecticidal portions thereof; or
2) a crystal protein from Bacillus thuringiensis or a portion thereof which is insecticidal in the presence of a second other crystal protein from Bacillus thuringiensis or a portion thereof, such as the binary toxin made up of the Cy34 and Cy35 crystal proteins; or
3) a hybrid insecticidal protein comprising parts of two different insecticidal crystal proteins from Bacillus thuringiensis, such as a hybrid of the proteins of 1) above or a hybrid of the proteins of 2) above, e.g. the Cry1A.105 protein produced by corn event MON98034 (WO 2007/027777); or
4) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes induced into the encoding DNA during cloning or transformation, such as the Cry3Bb1 protein in corn events MON863 or MON88017, or the Cry3A protein in corn event MIR 604;
5) an insecticidal secreted protein from Bacillus thuringiensis or Bacillus cereus, or an insecticidal portion thereof, such as the vegetative insecticidal proteins (VIP) listed at:
   http://www.lifesci.sussex.ac.uk/Home/Neil_Crickmore/Bt/vip.html, e.g. proteins from the VIP3Aa protein class;
6) a secreted protein from Bacillus thuringiensis or Bacillus cereus which is insecticidal in the presence of a second secreted protein from Bacillus thuringiensis or B. cereus, such as the binary toxin made up of the VIP1A and VIP2A proteins; or
7) a hybrid insecticidal protein comprising parts from different secreted proteins from Bacillus thuringiensis or Bacillus cereus, such as a hybrid of the proteins in 1) above or a hybrid of the proteins in 2) above; or
8) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes induced into the encoding DNA during cloning or transformation (while still encoding an insecticidal protein), such as the VIP3Aa protein in cotton event COT 102.

Of course, insect-resistant transgenic plants, as used herein, also include any plant comprising a combination of genes encoding the proteins of any one of the above classes 1 to 8. In one embodiment, an insect-resistant plant contains more than one transgene encoding a protein of any one of the above classes 1 to 8, to expand the range of target insect species affected or to delay insect resistance development to the plants, by using different proteins insecticidal to the same target insect species but having a different mode of action, such as binding to different receptor binding sites in the insect.

Plants or plant varieties (obtained by plant biotechnology methods such as genetic engineering) which may also be treated according to the invention are tolerant to abiotic stresses. Such plants can be obtained by genetic transformation, or by selection of plants containing a mutation imparting such stress resistance. Particularly useful stress tolerance plants include:
a. plants which contain a transgene capable of reducing the expression and/or the activity of the poly(ADP-ribose)polymerase (PARP) gene in the plant cells or plants.
b. plants which contain a stress tolerance-enhancing transgene capable of reducing the expression and/or the activity of the PARG-encoding genes of the plants or plant cells.
c. plants which contain a stress tolerance-enhancing transgene coding for a plant-functional enzyme of the nicotinamide adenine dinucleotide salvage biosynthesis pathway, including nicotinamidase, nicotinate phosphoribosyltransferase, nicotinic acid mononucleotide adenyltransferase, nicotinamide adenine dinucleotide synthetase or nicotinamide phosphoribosyltransferase.

Plants or plant varieties (obtained by plant biotechnology methods such as genetic engineering) which may also be treated according to the invention show altered quantity, quality and/or storage stability of the crop product and/or altered properties of specific ingredients of the crop product such as:
1) transgenic plants which synthesize a modified starch, which in its physical-chemical characteristics, in particular the amylose content or the amylose/amylopectin ratio, the degree of branching, the average chain length, the side chain distribution, the viscosity behaviour, the gelling strength, the starch grain size and/or the starch grain morphology, is changed in comparison with the synthesized starch in wild type plant cells or plants, so that this modified starch is better suited for special applications.
2) transgenic plants which synthesize non-starch carbohydrate polymers or which synthesize non-starch carbohydrate polymers with altered properties in comparison to wild type plants without genetic modification. Examples are plants which produce polyfructose, especially of the inulin and levan type, plants which produce alpha-1,4-glucans, plants which produce alpha-1,6 branched alpha-1,4-glucans, and plants producing alternan.
3) transgenic plants which produce hyaluronan.

Plants or plant varieties (obtained by plant biotechnology methods such as genetic engineering) which may also be treated according to the invention are plants, such as cotton plants, with altered fibre characteristics. Such plants can be obtained by genetic transformation, or by selection of plants containing a mutation imparting such altered fibre characteristics and include:
a) plants, such as cotton plants which contain an altered form of cellulose synthase genes,
b) plants, such as cotton plants which contain an altered form of rsw2 or rsw3 homologous nucleic acids;
c) plants, such as cotton plants, with an increased expression of sucrose phosphate synthase;
d) plants, such as cotton plants, with an increased expression of sucrose synthase;
e) plants, such as cotton plants, wherein the timing of the plasmodesmatal gating at the basis of the fibre cell is altered, e.g. through downregulation of fibre-selective β-1,3-glucanase;
f) plants, such as cotton plants, which have fibres with altered reactivity, e.g. through the expression of the N-acetylglucosaminetransferase gene including nodC and chitin synthase genes.

Plants or plant varieties (obtained by plant biotechnology methods such as genetic engineering) which may also be treated according to the invention are plants, such as oilseed rape or related Brassica plants, with altered oil profile characteristics. Such plants can be obtained by genetic transformation or by selection of plants containing a mutation imparting such altered oil characteristics and include:
a) plants, such as oilseed rape plants, which produce oil having a high oleic acid content;
b) plants, such as oilseed rape plants, which produce oil having a low linolenic acid content;
c) plants, such as oilseed rape plants, which produce oil having a low level of saturated fatty acids.

Particularly useful transgenic plants which may be treated according to the invention are plants which comprise one or more genes which encode one or more toxins are the following which are sold under the trade names YIELD GARD® (for example maize, cotton, soya beans), KnockOut® (for example maize), BiteGard® (for example maize), BT-Xtra® (for example maize), StarLink® (for example maize), Bollgard® (cotton), Nucotn® (cotton), Nucotn 33B® (cotton), NatureGard® (for example maize), Protecta® and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya beans), Liberty Link® (tolerance to phosphinothricin, for example oilseed rape), IMI® (tolerance to imidazolinone) and SCS® (tolerance to sylphonylurea, for example maize) Herbicide-resistant plants (plants bred in a conventional manner for herbicide tolerance) which may be mentioned include the varieties sold under the name Clearfield® (for example maize)

Particularly useful transgenic plants which may be treated according to the invention are plants containing transformation events, or a combination of transformation events, that are listed for example in the databases for various national or regional regulatory agencies (see for example http://gmoinfo.jrc.it/gmp_browse.aspx and http://www.agbios.com/dbase.php).

The active compounds or compositions according to the invention may furthermore be employed in the protection of materials for protecting industrial materials against attack and destruction by undesired microorganisms such as, for example, fungi.

In the present context, industrial materials are understood as meaning nonliving materials which have been prepared for use in industry. Industrial materials which are intended to be protected by active compounds according to the invention from microbial change or destruction can be, for example, glues, sizes, paper, wall card and board, textiles, carpets, leather, wood, paints and plastic articles, cooling lubricants and other materials which are capable of being attacked or decomposed by microorganisms. Other materials to be protected and which can be adversely affected by the multiplication of microorganisms which may be mentioned within the scope are parts of production plants and buildings, for example cooling water circuits, cooling and heating systems and aeration and air-conditioning units. Industrial materials which may be mentioned by preference within the scope of the present invention are glues, sizes, paper and boards, leather, wood, paints, cooling lubricants and heat-transfer fluids, especially preferably wood. The active compounds or compositions according to the invention can prevent disadvantageous effects such as wilting, decay, discolouration, decolouration or mould development. Moreover, the compounds according to the invention can be employed for protecting objects against being covered with growth, in particular ships' hulls, sieves, nets, buildings, jetties and signal units, which come into contact with seawater or brackish water.

The method according to the invention for controlling unwanted fungi can also be employed for protecting storage goods. Here, storage goods are to be understood as meaning natural substances of vegetable or animal origin or processed products thereof of natural origin, for which long-term protection is desired. Storage goods of vegetable origin, such as, for example, plants or plant parts, such as stems, leaves, tubers, seeds, fruits, grains, can be protected in the freshly harvested state or after processing by (pre)drying, moistening, comminuting, grinding, pressing or roasting. Storage goods also include timber, both unprocessed, such as construction timber, electricity poles and barriers, or in the form of finished products, such as furniture. Storage goods of animal origin are, for example, pelts, leather, furs and hairs. The active compounds according to the invention can prevent disadvantageous effects, such as rotting, decay, discolouration, decolouration or the development of mould.

Some pathogens of fungal diseases which can be treated according to the invention may be mentioned, by way of example, but not by way of limitation:

Diseases caused by powdery mildew pathogens, such as, for example, Blumeria species, such as, for example, Blumeria graminis; Podosphaera species, such as, for example, Podosphaera leucotricha; Sphaerotheca species, such as, for example, Sphaerotheca fuliginea; Uncinula species, such as, for example, Uncinula necator;

Diseases caused by rust disease pathogens, such as, for example, Gymnosporangium species, such as, for example, Gymnosporangium sabinae; Hemileia species, such as, for example, Hemileia vastatrix; Phakopsora species, such as, for example, Phakopsora pachyrhizi and Phakopsora meibomiae; Puccinia species, such as, for example, Puccinia recondita or Puccinia triticina; Uromyces species, such as, for example, Uromyces appendiculatus;

Diseases caused by pathogens from the group of the Oomycetes, such as, for example, Bremia species, such as, for example, Bremia lactucae; Peronospora species, such as, for example, Peronospora pisi or P. brassicae; Phytophthora species, such as, for example, Phytophthora infestans; Plasmopara species, such as, for example, Plasmopara viticola; Pseudoperonospora species, such as, for example, Pseudoperonospora humuli or Pseudoperonospora cubensis; Pythium species, such as, for example, Pythium ultimum;

Leaf blotch diseases and leaf wilt diseases caused, for example, by Alternaria species, such as, for example, Alternaria solani; Cercospora species, such as, for example, Cercospora beticola; Cladiosporum species, such as, for example, Cladiosporium cucumerinum; Cochliobolus species, such as, for example, Cochliobolus sativus (conidia form: Drechslera, Syn: Helminthosporium); Colletotrichum species, such as, for example, Colletotrichum lindemuthanium; Cycloconium species, such as, for example, Cycloconium oleaginum; Diaporthe species, such as, for example, Diaporthe citri; Elsinoe species, such as, for example, Elsinoe fawcettii; Gloeosporium species, such as, for example, Gloeosporium laeticolor; Glomerella species, such as, for example, Glomerella cingulata; Guignardia species, such as, for example, Guignardia bidwelli; Leptosphaeria species, such as, for example, Leptosphaeria maculans; Magnaporthe species, such as, for example, Magnaporthe grisea; Microdochium species, such as, for example, Microdochium nivale; Mycosphaerella species, such as, for example, Mycosphaerella graminicola and M. fijiensis; Phaeosphaeria species, such as, for example, Phaeosphaeria nodorum; Pyrenophora species, such as, for example, Pyrenophora teres; Ramularia species, such as, for example, Ramularia collo-cygni; Rhynchosporium species, such as, for example, Rhynchosporium secalis; Septoria species, such as, for example, Septoria apii; Typhula species, such as, for example, Typhula incarnata; Venturia species, such as, for example, Venturia inaequalis;

Root and stem diseases caused, for example, by Corticium species, such as, for example, Corticium graminearum; Fusarium species, such as, for example, Fusarium oxysporum; Gaeumannomyces species, such as, for example, Gaeumannomyces graminis; Rhizoctonia species, such as, for example, Rhizoctonia solani; Tapesia species, such as, for example, Tapesia acuformis; Thielaviopsis species, such as, for example, Thielaviopsis basicola;

Ear and panicle diseases (including maize cobs) caused, for example, by Alternaria species, such as, for example, Alternaria spp.; Aspergillus species, such as, for example, Aspergillus flavus; Cladosporium species, such as, for example, Cladosporium cladosporioides; Claviceps species, such as, for example, Claviceps purpurea; Fusarium species, such as, for example, Fusarium culmorum; Gibberella species, such as, for example, Gibberella zeae; Monographella species, such as, for example, Monographella nivalis; Septoria species, such as, for example, Septoria nodorum;

Diseases caused by smut fungi, such as, for example, Sphacelotheca species, such as, for example, Sphacelotheca reiliana; Tilletia species, such as, for example, Tilletia caries, T. controversa; Urocystis species, such as, for example, Urocystis occulta; Ustilago species, such as, for example, Ustilago nuda, U. nuda tritici;

Fruit rot caused, for example, by Aspergillus species, such as, for example, Aspergillus flavus; Botrytis species, such as, for example, Botrytis cinerea; Penicillium species, such as, for example, Penicillium expansum and P. purpurogenum; Sclerotinia species, such as, for example, Sclerotinia sclerotiorum;

Verticilium species, such as, for example, Verticilium alboatrum;

Seed- and soil-borne rot and wilt diseases, and also diseases of seedlings, caused, for example, by Fusarium species, such as, for example, Fusarium culmorum; Phytophthora species, such as, for example, Phytophthora cactorum; Pythium species, such as, for example, Pythium ultimum; Rhizoctonia species, such as, for example, Rhizoctonia solani; Sclerotium species, such as, for example, Sclerotium rolfsii;

Cancerous diseases, galls and witches' broom caused, for example, by Nectria species, such as, for example, Nectria galligena;

Wilt diseases caused, for example, by Monilinia species, such as, for example, Monilinia laxa;

Deformations of leaves, flowers and fruits caused, for example, by Taphrina species, such as, for example, Taphrina deformans;

Degenerative diseases of woody plants caused, for example, by Esca species, such as, for example, Phaemoniella clamydospora and Phaeoacremonium aleophilum and Fomitiporia mediterranea;

Diseases of flowers and seeds caused, for example, by Botrytis species, such as, for example, Botrytis cinerea;

Diseases of plant tubers caused, for example, by Rhizoctonia species, such as, for example, Rhizoctonia solani; Helminthosporium species, such as, for example, Helminthosporium solani;

Diseases caused by bacteriopathogens, such as, for example, Xanthomonas species, such as, for example, Xanthomonas campestris pv. oryzae; Pseudomonas species, such as, for example, Pseudomonas syringae pv. lachrymans; Erwinia species, such as, for example, Erwinia amylovora.

Preference is given to controlling the following diseases of soya beans:

Fungal diseases on leaves, stems, pods and seeds caused, for example, by alternaria leaf spot (Alternaria spec. atrans tenuissima), anthracnose (Colletotrichum gloeosporoides dematium var. truncatum), brown spot (Septoria glycines), cercospora leaf spot and blight (Cercospora kikuchii), choanephora leaf blight (Choanephora infundibulifera trispora (Syn.)), dactuliophora leaf spot (dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi, Phakopsora meibomiae), scab (Sphaceloma glycines), stemphylium leaf blight (Stemphylium botryosum), target spot (Corynespora cassiicola).

Fungal diseases on roots and the stem base caused, for example, by black root rot (Calonectria crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmopspora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidermatum, Pythium irregulare, Pythium debaryanum, Pythium myriotylum, Pythium ultimum), rhizoctonia root rot, stem decay, and damping-off (Rhizoctonia solani), sclerotinia stem decay (Sclerotinia sclerotiorum), sclerotinia Southern blight (Sclerotinia rolfsii), thielaviopsis root rot (Thielaviopsis basicola).

Microorganisms which can bring about degradation or modification of the industrial materials and which may be mentioned are, for example, bacteria, fungi, yeasts, algae and slime organisms. The active compounds according to the invention are preferably active against fungi, in particular moulds, wood-discolouring and wood-destroying fungi (Basidiomycetes) and also against slime organisms and algae. Microorganisms of the following genera may be mentioned by way of example: Alternaria, such as Alternaria tenuis; Aspergillus, such as Aspergillus niger; Chaetomium, such as Chaetomium globosum; Coniophora, such as Coniophora puetana; Lentinus, such as Lentinus tigrinus; Penicillium, such as Penicillium glaucum; Polyporus, such as Polyporus versicolor; Aureobasidium, such as Aureobasidium pullulans; Sclerophoma, such as Sclerophoma pityophila; Trichoderma, such as Trichoderma viride; Escherichia, such as Escherichia coli; Pseudomonas, such as Pseudomonas aeruginosa; Staphylococcus, such as Staphylococcus aureus.

In addition, the compounds of the formula (I) according to the invention also have very good antimycotic activity. They have a very broad antimycotic activity spectrum, in particular against dermatophytes and yeasts, moulds and diphasic fungi (for example against Candida species, such as Candida albicans, Candida glabrata) and Epidermophyton floccosum, Aspergillus species such as Aspergillus niger and Aspergillus fumigatus, Trichophyton species such as Trichophyton mentagrophytes, Microsporon species such as Microsporon canis and audouinii. The enumeration of these fungi does by no means limit the mycotic spectrum which can be covered, but is only for illustration.

Accordingly, the active compounds according to the invention can be used both in medical and in non-medical applications.

When employing the active compounds according to the invention as fungicides, the application rates may vary within a substantial range, depending on the type of application. The application rate of the active compounds according to the invention is
when treating plant parts, for example leaves: from 0.1 to 10 000 g/ha, preferably from 10 to 1000 g/ha, particularly preferably from 50 to 300 g/ha (when the application is carried out by watering or dropwise, it may even be possible to reduce the application rate, in particular when inert substrates such as rock wool or perlite are used);
when treating seed: from 2 to 200 g per 100 kg of seed, preferably from 3 to 150 g per 100 kg of seed, especially preferably from 2.5 to 25 g per 100 kg of seed, very especially preferably from 2.5 to 12.5 g per 100 kg of seed;
when treating the soil: from 0.1 to 10 000 g/ha, preferably from 1 to 5000 g/ha.

These application rates are mentioned only by way of example and not by way of limitation in the sense of the invention.

The active compounds or compositions according to the invention can thus be employed for protecting plants for a certain period of time after treatment against attack by the pathogens mentioned. The period for which protection is provided extends generally for 1 to 28 days, preferably 1 to 14 days, particularly preferably 1 to 10 days, very particularly preferably 1 to 7 days after the treatment of the plants with the active compounds, or up to 200 days after the treatment of seed.

In addition, by the treatment according to the invention it is possible to reduce the mycotoxin content in the harvested material and the foodstuff and feedstuff prepared therefrom. Particular, but not exclusive, mention may be made here of the following mycotoxins: deoxynivalenol (DON), nivalenol, 15-Ac-DON, 3-Ac-DON, T2- and HT2-toxin, fumonisine, zearalenon, moniliformin, fusarin, diaceotoxyscirpenol (DAS), beauvericin, enniatin, fusaroproliferin, fusarenol, ochratoxins, patulin, ergot alkaloids and aflatoxins produced, for example, by the following fungi: Fusarium spec., such as Fusarium acuminatum, F. avenaceum, F. crookwellense, F. culmorum, F graminearum (Gibberella zeae), F. equiseti, F. fujikoroi, F. musarum, F. oxysporum, F. proliferatum, F. poae, F. pseudograminearum, F. sambucinum, F. scirpi, F. semitectum, F. solani, F. sporotrichoides, F. langsethiae, F. subglutinans, F. tricinctum, F. verticillioides, inter alia, and also by Aspergillus spec., Penicillium spec., Claviceps purpurea, Stachybotrys spec. inter alia.

The abovementioned plants can be treated especially advantageously in accordance with the invention with the compounds of the general formula (I) the compositions according to the invention. The preferred ranges indicated above for the active compounds or compositions also apply to the treatment of these plants. The treatment of plants with the compounds or compositions mentioned specifically in the present text should be especially emphasized.

The preparation and use of the active compounds according to the invention emerges from the following examples.

### PREPARATION EXAMPLES

Compound I-178:

### 4,6-Dihydroxy-5-methoxy-1-(4-methoxybenzyl)-3-(2-methylheptanoyl)pyridin-2(1H)-one

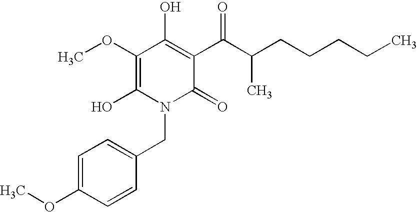

At 0° C., 4.76 ml (55.5 mol) of oxalyl chloride were added dropwise to a suspension of 3.69 g (27.5 mmol) of methoxymalonic acid and two drops of dimethylformide in 30 ml of dry dichloromethane, and the mixture was stirred for 24 h. The solution obtained in this manner was added dropwise to a suspension of 7.0 g (22.9 mmol) of N-(4-methoxybenzyl)-4-methyl-3-oxononanamide in 10 ml of dry toluene, and the mixture was stirred for 72 h. The reaction mixture was extracted twice with in each case 60 ml of 1N sodium hydroxide solution. The aqueous phases were combined, washed with dichloromethane and adjusted to pH 0 using concentrated hydrochloric acid. The product was extracted from the acidic solution using ethyl acetate. The organic phase was dried, filtered and concentrated. The product obtained in this manner was used without further purification for the next step.

Yield: 4.80 g (52% of theory).

¹H NMR (CDCl₃): δ=0.87 (t, 3H), 1.19 (d, 3H), 1.23-1.35 (m, 6H), 1.42 (m, 1H), 1.76 (m, 1H), 3.77-3.82 (m, 1H), 3.78 (s, 3H), 3.89 (s, 3H), 5.13 (s, 2H), 6.83 (d, 2H), 7.45 (d, 2H).

LC-MS: m/z=404 [M+H]⁺.

Compound I-186:

### 4-Hydroxy-5,6-dimethoxy-1-(4-methoxybenzyl)-3-(2-methylheptanoyl)pyridin-2(1H)-one

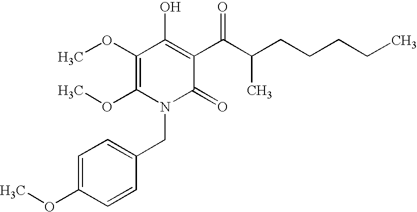

A little at a time 1.54 g (10.4 mmol) of solid trimethyloxonium tetrafluoroborate were added to a solution of 3.00 g (7.44 mmol) of 4,6-dihydroxy-5-methoxy-1-(4-methoxybenzyl)-3-(2-methyl-heptanoyl)pyridin-2(1H)-one (I-178) and 1.30 ml (7.44 mmol) of N,N-diisopropylethylamine in 50 ml of dry dichloromethane, and the mixture was stirred at room temperature. The reaction mixture was washed with 1N hydrochloric acid, and the organic phase was dried, filtered and concentrated. The residue was purified by column chromatography (silica gel, mobile phase: cyclohexane/ethyl acetate 10:0 to 10:5).

Yield: 1.50 g (48% of theory).

¹H NMR (CDCl₃): δ=0.87 (t, 3H), 1.16 (d, 3H), 1.24-1.35 (m, 6H), 1.65-1.80 (m, 2H), 3.76 (s, 3H), 3.78 (s, 3H), 4.10 (s, 3H), 4.18 (q, 1H), 5.14 (s, 2H), 6.83 (d, 2H), 7.25 (d, 2H).

LC-MS: m/z=418 [M+H]⁺.

Compound I-198:

### 4-Hydroxy-5,6-dimethoxy-3-(2-methylheptanoyl)pyridin-2(1H)-one

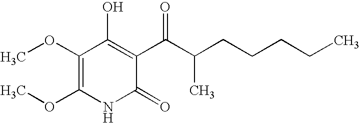

100 mg of palladium catalyst (10% on activated carbon) were added to a solution of 970 mg (2.33 mmol) of 4-hydroxy-5,6-dimethoxy-1-(4-methoxybenzyl)-3-(2-methylheptanoyl)pyridin-2(1H)-one in 80 ml of tetrahydrofuran, and the mixture was hydrogenated at atmospheric pressure under an atmosphere of hydrogen for 24 h. The reaction mixture was filtered through Celite and the residue was titrated with cyclohexane/ethyl acetate. The solid was filtered off with suction and dried under high vacuum.

Yield: 530 mg (60% of theory).

¹H NMR (DMSO-d₆): δ=0.85 (t, 3H), 1.08 (d, 3H), 1.20-1.30 (m, 7H), 1.69 (m, 1H), 3.62 (s, 3H), 3.88 (m, 1H), 3.92 (s, 3H).

LC-MS: m/z=298 [M+H]⁺.

Compound I-1:

### 4-Hydroxy-5,6-dimethoxy-3-[(4E)-2-methylhex-4-enoyl]pyridin-2(1H)-one

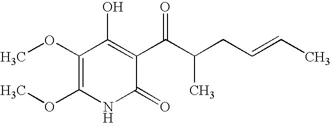

A solution of 33 mg (0.07 mmol) of 1-(2,4-dimethoxybenzyl)-4-hydroxy-5,6-dimethoxy-3-[(4E)-2-methylhex-4-enoyl]pyridin-2(1H)-one in 3 ml of trifluoroacetic acid was stirred at room temperature for 12 h. The mixture was then diluted with toluene and concentrated under reduced pressure. The residue was dried under high vacuum.

Yield: 21 mg (88% of theory).

¹H NMR (DMSO-d₆): δ=1.05 (d, 3H), 1.59 (d, 3H), 1.96 (m, 1H), 2.35 (m, 1H), 3.61 (s, 3H), 3.84 (m, 1H), 3.92 (s, 3H), 5.34-5.43 (m, 2H).

LC-MS: m/z=282 [M+H]⁺.

Compound I-9:

### 4,6-Dihydroxy-5-methoxy-1-(4-methoxybenzyl)-3-propanoylpyridin-2(1H)-one

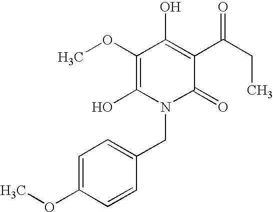

At 0° C., 47.6 ml (555 mmol) of oxalyl chloride were added dropwise to a suspension of 36.9 g (275 mmol) of methoxymalonic acid and two drops of dimethylformamide in 500 ml of dry dichloromethane, and the mixture was stirred for 24 h. The solution obtained in this manner was added dropwise to a suspension of 54.0 g (230 mmol) of N-(4-methoxybenzyl)-3-oxopentaneamide in 150 ml of dry toluene, and the mixture was stirred for 72 h. The reaction mixture was extracted twice with in each case 600 ml of 1N sodium hydroxide solution. The aqueous phases were combined, washed with dichloromethane and adjusted to pH 0 using concentrated hydrochloric acid. The product was extracted with ethyl acetic acid. The organic phase was dried, filtered and concentrated. The product obtained in this manner was used without further purification for the next step.

Yield: 42.6 g (56% of theory).

¹H NMR (CDCl₃): δ=1.22 (t, 3H), 3.01 (q, 2H), 3.78 (s, 3H), 3.89 (s, 3H), 5.12 (s, 2H), 6.82 (d, 2H), 7.43 (d, 2H).

LC-MS: m/z=334 [M+H]⁺.

Compound I-48:

### 4-Hydroxy-5,6-dimethoxy-1-(4-methoxybenzyl)-3-propanoylpyridin-2(1H)-one

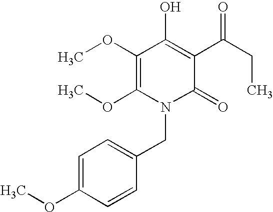

A little at a time, 466 mg (3.15 mmol) of solid trimethyloxonium tetrafluoroborate were added to a solution of 1.00 g (3.00 mmol) of 4,6-dihydroxy-5-methoxy-1-(4-methoxybenzyl)-3-propanoyl-pyridin-2(1H)-one (I-9) and 0.52 ml (3.00 mmol) of N,N-diisopropylethylamine in 15 ml of dry di-chloromethane, and the mixture was stirred at room temperature. The reaction mixture was washed with 1N hydrochloric acid and the organic phase was dried, filtered and concentrated. The residue was subjected to fractional crystallization from ethyl acetate and methanol.

Yield: 640 mg (61% of theory).

¹H-NMR (CDCl₃): δ=1.17 (t, 3H), 3.21 (q, 2H), 3.76 (s, 3H), 3.78 (s, 3H), 4.12 (s, 3H), 5.12 (s, 2H), 6.84 (d, 2H), 7.25 (d, 2H).

LC-MS: m/z=348 [M+H]⁺.

Compound I-258:

### 3-(2,4-Dimethylpent-4-enoyl)-4-hydroxy-5,6-dimethoxy-1-(4-methoxybenzyl)pyridin-2(1H)-one

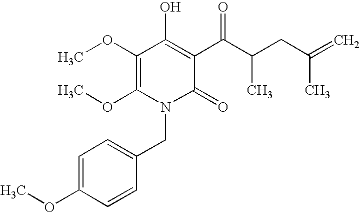

At −78° C., 2.13 g (19.0 mmol) of solid potassium tert-butoxide were added a little at a time to a solution of 3.00 g (8.64 mmol) of 4-hydroxy-5,6-dimethoxy-1-(4-methoxybenzyl)-3-propanoylpyridin-2(1H)-one (I-48) in 75 ml of dry tetrahydrofuran, and the mixture was stirred at −78° C. for 10 minutes. 5.83 g (43.2 mmol) of 3-bromo-2-methyl-1-propene were then added dropwise. The mixture was stirred for 24 hours and slowly warmed to room temperature in the process. The reaction mixture was poured into 50 ml of ice-cold 1N hydrochloric acid and the solution was extracted three times with dichloromethane. The organic phases were dried, filtered and concentrated. The residue was purified by column chromatography (silica gel, mobile phase: cyclohexane/ethyl acetate 100:0 to 100:3).

Yield: 1.83 g (51% of theory).

¹H NMR (CDCl₃): δ=1.14 (d, 3H), 1.77 (s, 3H), 2.04 (m, 1H), 2.54 (m, 1H), 3.75 (s, 3H), 3.79 (s, 3H), 4.10 (s, 3H), 4.46 (m, 1H), 4.71 (s, 1H), 4.75 (s, 1H), 5.14 (s, 2H), 6.84 (d, 2H), 7.25 (d, 2H).

LC-MS: m/z=402 [M+H]⁺.

Compound I-138:

### 3-(2,4-Dimethylpentanoyl)-4-hydroxy-5,6-dimethoxypyridin-2(1H)-one

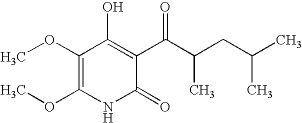

46.2 mg of palladium catalyst (10% on activated carbon) were added to a solution of 430 mg (1.07 mmol) of 3-(2,4-dimethylpent-4-enoyl)-4-hydroxy-5,6-dimethoxy-1-(4-methoxybenzyl)pyridin-2(1H)-one (I-258) in 10 ml of tetrahydrofuran, and the mixture was hydrogenated at atmospheric pressure under an atmosphere of hydrogen for 24 h. The reaction mixture was filtered through Celite and the residue was titrated with cyclohexane/ethyl acetate. The solid was filtered off with suction and dried under high vacuum.

Yield: 225 mg (74% of theory).

¹H NMR (CDCl₃): δ=0.89 (d, 3H), 0.90 (d, 3H), 1.14 (d, 3H), 1.22 (m, 1H), 1.62 (m, 1H), 1.72 (m, 1H), 3.83 (s, 3H), 3.99 (m, 1H), 4.09 (s, 3H).

LC-MS: m/z=284 [M+H]⁺.

Compound I-172:

### 3-[3-(2,6-Dichlorophenyl)-2-methylpropanoyl]-4-hydroxy-5,6-dimethoxy-1-(4-methoxybenzyl)pyridin-2(1H)-one

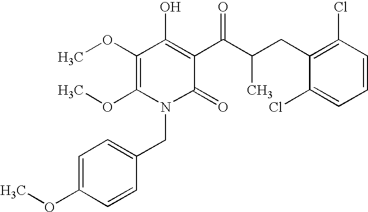

At −78° C., 0.95 ml (0.95 mmol) of a 1N solution of sodium bis(trimethylsilyl)amide in tetrahydrofuran was added dropwise to a solution of 150 mg (0.43 mmol) of 4-hydroxy-5,6-dimethoxy-1-(4-methoxybenzyl)-3-propanoylpyridin-2(1H)-one (I-48) in 5 ml of dry tetrahydrofuran, and the mixture was stirred at −78° C. for 20 minutes. A solution of 518 mg (2.16 mmol) of 2,5-dichlorobenzyl bromide in 1 ml of dry tetrahydrofuran was then added dropwise. The mixture was stirred for 24 hours and slowly warmed to room temperature in the process. The reaction mixture was poured into 50 ml of ice-cold 1N hydrochloric acid and the solution was extracted three times with dichloromethane. The organic phases were dried, filtered and concentrated. The residue was purified by column chromatography (silica gel, mobile phase: cyclohexane/ethyl acetate 100:0 to 100:4).

Yield: 150 mg (69% of theory).

¹H-NMR (CDCl₃): δ=1.21 (d, 3H), 3.18 (dd, 1H), 3.35 (dd, 1H), 3.77 (s, 3H), 3.79 (s, 3H), 4.10 (s, 3H), 4.65 (m, 1H), 5.05-5.11 (m, 2H), 6.81 (d, 2H), 7.05 (t, 1H), 7.20-7.25 (m, 4H).

LC-MS: m/z=506 {[M(Cl³⁵)+H]⁺, 2 Cl}.

Compound I-174:

### 3-[3-(2,6-Dichlorophenyl)-2-methylpropanoyl]-4-hydroxy-5,6-dimethoxypyridin-2(1H)-one

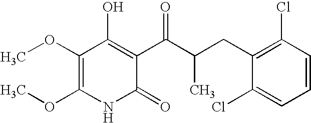

A solution of 110 mg (0.22 mmol) of 3-[3-(2,6-dichlorophenyl)-2-methylpropanoyl]-4-hydroxy-5,6-dimethoxy-1-(4-methoxybenzyl)pyridin-2(1H)-one (I-172) in 5 ml of trifluoroacetic acid was heated under reflux for 20 h. The solvent was then removed under reduced pressure and the residue was titrated with cyclohexane/ethyl acetate. The solid was filtered off with suction and dried under high pressure.

Yield: 83 mg (98% of theory).

¹H NMR (CDCl₃): δ=1.20 (d, 3H), 3.20 (dd, 1H), 3.34 (dd, 1H), 3.79 (s, 3H), 4.04 (s, 3H), 4.38 (m, 1H), 7.08 (t, 1H), 7.27 (d, 2H).

LC-MS: m/z=386 {[M(Cl³⁵)+H]⁺, 2 Cl}.

Compound I-238:

### Methyl 4,6-dihydroxy-5-methoxy-1-(4-methoxybenzyl)-2-oxo-1,2-dihydropyridine-3-carboxylate

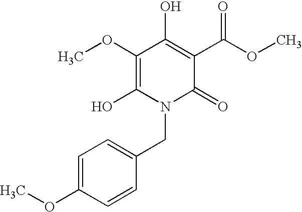

At room temperature, 13.4 ml (153 mmol) of oxalyl chloride were added dropwise to a suspension of 10.2 g (75 9 mmol) of methoxymalonic acid and two drops of dimethylformamide in 1000 ml of dichloromethane. The solution obtained in this manner was added dropwise to a suspension of 15.0 g (63.2 mmol) of methyl 3-[(4-methoxybenzyl)amino]-3-oxopropionoate in 100 ml of dry toluene, and the mixture was stirred for 72 h. The reaction mixture was extracted twice with in each case 300 ml of 1N sodium hydroxide solution. The aqueous phases were combined, washed with dichloromethane and adjusted to pH 0 using concentrated hydrochloric acid. The product was extracted from the acidic solution using ethyl acetate. The organic phase was dried, filtered and concentrated. The residue was subjected to fractional recrystallization from ethyl acetate.

Yield: 5.44 g (36% of theory).

¹H NMR (CDCl₃): δ=3.77 (s, 3H), 3.84 (s, 3H), 4.02 (s, 3H), 5.21 (s, 2H), 6.83 (d, 2H), 7.43 (d, 2H).

LC-MS: m/z=336 [M+H]⁺.

Compound I-100:

### Methyl 4-hydroxy-5,6-dimethoxy-1-(4-methoxybenzyl)-2-oxo-1,2-dihydropyridine-3-carboxylate

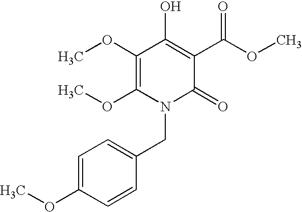

A little at a time, 5.73 g (38.8 mol) of solid trimethyloxonium tetrafluoroborate were added to a solution of 10.0 g (29.8 mmol) of methyl 4,6-dihydroxy-5-methoxy-1-(4-methoxybenzyl)-2-oxo-1,2-dihydropyridine-3-carboxylate (I-238) and 5.71 ml (32.8 mmol) of N,N-diisopropylethylamine in 100 ml of dry dichloromethane, and the mixture was stirred at room temperature. After 24 h, a further 5.73 g (38.8 mmol) of solid trimethyloxonium tetrafluoroborate were added a little at a time, and the mixture was stirred at room temperature for 24 h. The reaction mixture was washed with 1N hydrochloric acid and the organic phase was dried, filtered and concentrated. The residue was subjected to fractional recrystallization from ethyl acetate.

Yield: 3.30 g (41% of theory).

¹H NMR (CDCl₃): δ=3.75 (s, 3H), 3.78 (s, 3H), 3.97 (s, 3H), 4.11 (s, 3H), 5.13 (s, 2H), 6.82 (d, 2H), 7.30 (d, 2H).

LC-MS: m/z=350 [M+H]⁺.

Compound I-43:

### 4-Hydroxy-5,6-dimethoxy-1-(4-methoxybenzyl)-2-oxo-1,2-dihydropyridine-3-carboxylic acid

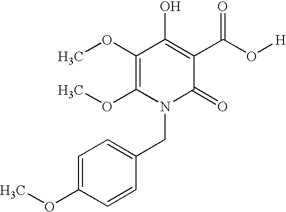

300 mg (0.86 mmol) of methyl 4-hydroxy-5,6-dimethoxy-1-(4-methoxybenzyl)-2-oxo-1,2-dihydropyridine-3-carboxylate (I-100) and 22.6 mg (0.96 ml) lithium hydroxide were dissolved in 5 ml of tetrahydrofuran/water 3:1 and stirred at room temperature. After 6 h, a further 22.6 mg (0.95 mmol) of lithium hydroxide were added, and the mixture was stirred at room temperature for 18 h. After acidification with 50 ml of 1N hydrochloric acid, the mixture was extracted three times with dichloromethane. The organic phases were combined, dried, filtered and concentrated. The residue was titrated with cyclohexane and methanol and then purified by column chromatography (silica gel, mobile phase: cyclohexane/dichloromethane 10:1 to 10:6).

Yield: 83 mg (29% of theory).

¹H-NMR (DMSO-d₆): δ=3.73 (s, 3H), 3.75 (s, 3H), 4.16 (s, 3H), 5.13 (s, 2H), 6.89 (d, 2H), 7.23 (d, 2H).

LC-MS: m/z=336 [M+H]⁺

Compound I-255:

### 4-Hydroxy-5,6-dimethoxy-1-(4-methoxybenzyl)pyridin-2(1H)-one

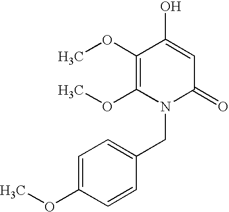

10 ml of a 5N sodium hydroxide solution were added to a solution of 390 mg (1.12 mmol) of methyl 4-hydroxy-5,6-dimethoxy-1-(4-methoxybenzyl)-2-oxo-1,2-dihydropyridine-3-carboxylate (I-100) in 8 ml THF, and the mixture was, with stirring, heated in a microwave (50 Watt, 70° C.) for 45 minutes. After cooling, the mixture was acidified with 50 ml of 2N hydrochloric acid and extracted three times with dichloromethane. The organic phases were combined, dried, filtered and concentrated.

Yield: 255 mg (78% of theory).

¹H NMR (CDCl₃): δ=3.78 (2s, 6H), 3.96 (s, 3H), 5.20 (s, 2H), 6.58 (s, 1H), 6.84 (d, 2H), 7.25 (d, 2H).

LC-MS: m/z=292 [M+H]⁺.

Compound I-95:

### 4-Hydroxy-5,6-dimethoxy-1-(4-methoxybenzyl)-2-oxo-N-[4′-(trifluoromethyl)biphenyl-2-yl]-1,2-dihydropyridine-3-carboxamide

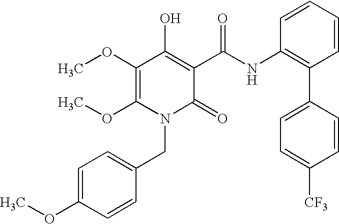

150 mg (0.43 mmol) of methyl 4-hydroxy-5,6-dimethoxy-1-(4-methoxybenzyl)-2-oxo-1,2-dihydropyridine-3-carboxylate (I-100), 101.8 mg (0.43 mmol) of 4′-(trifluoromethyl)biphenyl-2-amine and 40.8 mg (0.43 mmol) of 2-pyridone in 15 ml of toluene were heated under reflux for 24 h. The reaction mixture was concentrated to dryness and recrystallized from methanol.

Yield: 170 mg (71% of theory).

¹H NMR (DMSO-d⁶): δ=3.69 (s, 3H), 3.73 (s, 3H), 4.09 (s, 3H), 4.92, (s, 2H), 6.84 (d, 2H), 7.13 (d, 2H), 7.29-7.38 (m, 2H), 7.46 (t, 1H), 7.62 (d, 2H), 7.78 (d, 2H), 8.09 (d, 1H).

LC-MS: m/z=555 [M+H]⁺.

Compound I-98:

### 4-Hydroxy-5,6-dimethoxy-2-oxo-N-[4′-(trifluoromethyl)biphenyl-2-yl]-1,2-dihydropyridine-3-carboxamide

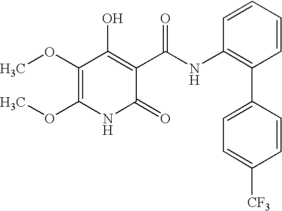

145 mg (0.26 mmol) of 4-hydroxy-5,6-dimethoxy-1-(4-methoxybenzyl)-2-oxo-N-[4′-(trifluoromethyl)biphenyl-2-yl]-1,2-dihydropyridine-3-carboxamide (I-95) in 8 ml of trifluoroacetic acid were heated under reflux for 20 h. The reaction mixture was concentrated to dryness, taken up in toluene and once more concentrated to dryness. This procedure was repeated two more times. The residue was titrated with warm methanol, filtered off with suction and dried under reduced pressure.

Yield: 88 mg (77% of theory).

¹H-NMR (CDCl₃): δ=3.79 (s, 3H), 4.11 (s, 3H), 7.25-7.29 (m, 2H), 7.43 (m, 1H), 7.54 (d, 2H), 7.68 (d, 2H), 8.10 (d, 1H).

LC-MS: m/z=435 [M+H]⁺.

Compound I-134:

### 4-Hydroxy-5,6-dimethoxy-1-(4-methoxybenzyl)-N-(2-methylbutyl)-2-oxo-1,2-dihydropyridine-3-carboxamide

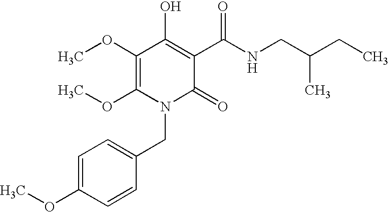

300 mg (0.86 mmol) of methyl 4-hydroxy-5,6-dimethoxy-1-(4-methoxybenzyl)-2-oxo-1,2-dihydropyridine-3-carboxylate (I-100), 74.9 mg (0.86 mmol) of 2-methylbutylamine and 81.7 mg (0.86 mmol) of 2-pyridone in 4 ml of toluene were heated to 130° C. in a microwave reactor (210 Watt) for 20 min. The reaction mixture was concentrated to dryness and the residue was purified by column chromatography (silica gel, mobile phase: cyclohexane/ethyl acetate 10:0 to 10:2).

Yield: 280 mg (77% of theory).

¹H NMR (CDCl₃): δ=0.91-0.98 (2t, 6H), 1.21 (m, 1H), 1.48 (m, 1H), 1.68 (m, 1H), 3.21 (m, 1H), 3.33 (m, 1H), 3.77 (s, 3H), 3.78 (s, 3H), 4.03 (s, 3H), 5.15 (s, 2H), 6.85 (d, 2H), 7.21 (d, 2H), 10.18 (br. t, 1H).

LC-MS: m/z=405 [M+H]⁺.

Compound I-144:

### 4-Hydroxy-5,6-dimethoxy-N-(2-methylbutyl)-2-oxo-1,2-dihydropyridine-3-carboxamide

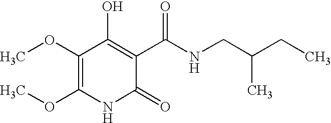

20 mg of palladium catalyst (10% on activated carbon) were added to a solution of 130 mg (0.32 mmol) of 4-hydroxy-5,6-dimethoxy-1-(4-methoxybenzyl)-N-(2-methylbutyl)-2-oxo-1,2-dihydropyridine-3-carboxamide (I-134) in 8 ml of tetrahydrofuran, and the mixture was hydrogenated at atmospheric pressure under an atmosphere of hydrogen for 24 h. The reaction mixture was filtered through Celite and the filtrate was concentrated. The residue was titrated with methanol/tetrahydrofuran 1:1, filtered off with suction and dried under high vacuum.

Yield: 57 mg (59% of theory).

¹H NMR (CDCl₃): δ=0.91-0.97 (m, 6H), 1.20-1.25 (m, 1H), 1.42-1.50 (m, 1H), 1.63-1.68 (m, 1H), 3.24 (m, 1H), 3.35 (m, 1H), 3.80 (s, 3H), 4.17 (s, 3H).

LC-MS: m/z=285 [M+H]⁺.

Compound I-314:

### N-Butyl-4-hydroxy-5,6-dimethoxy-1-(4-methoxybenzyl)-N-methyl-2-oxo-1,2-dihydropyridine-3-carboxamide

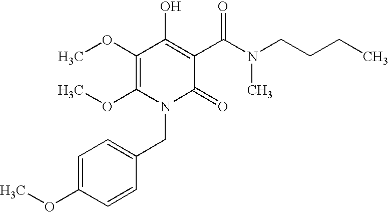

Under irradiation with microwaves (210 Watt), 50 mg (0.14 mmol) of methyl 4-hydroxy-5,6-dimethoxy-1-(4-methoxybenzyl)-2-oxo-1,2-dihydropyridine-3-carboxylate (I-100), 12.5 mg (0.14 mmol) of N-methylbutylamine and 13.6 mg (0.14 mmol) of 2-pyridone in 4 ml of toluene were heated at 150° C. for 30 min. The reaction mixture was concentrated to dryness and the residue was purified by RP-HPLC.

Yield: 32 mg (54% of theory).

¹H NMR (MeCN-d₃): δ=0.88 (t, 3H), 1.27 (m, 2H), 1.55 (m, 2H), 2.92 (s, 3H), 3.37 (t, 2H), 3.68 (s, 3H), 3.74 (s, 3H), 3.99 (s, 3H), 5.08 (s, 2H), 6.85 (d, 2H), 7.21 (d, 2H).

LC-MS: m/z=405 [M+H]⁺.

Compound I-248:

### N-Butyl-4-hydroxy-5,6-dimethoxy-N-methyl-2-oxo-1,2-dihydropyridine-3-carboxamide

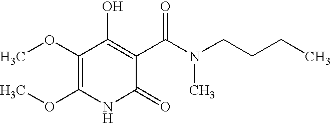

10 mg of palladium catalyst (10% on activated carbon) were added to a solution of 22 mg (54 μmol) of N-butyl-4-hydroxy-5,6-dimethoxy-1-(4-methoxybenzyl)-N-methyl-2-oxo-1,2-dihydropyridine-3-carboxamide (I-314) in 6 ml of tetrahydrofuran/methanol 1:1, and the mixture was hydrogenated at atmospheric pressure under an atmosphere of hydrogen for 24 h. The reaction mixture was filtered through Celite and the filtrate was concentrated.

Yield: 11 mg (68% of theory).

¹H-NMR (MeCN-d³): δ=0.87 (t, 3H), 1.26 (m, 2H), 1.53 (m, 2H), 2.91 (s, 3H), 3.33 (t, 2H), 3.69 (s, 3H), 3.87 (s, 3H).

LC-MS: m/z=285 [M+H]⁺.

Compound I-244:

### 4-Hydroxy-5,6-dimethoxy-1-(4-methoxybenzyl)-2-oxo-N-(2-phenylpropyl)-1,2-dihydropyridine-3-carboxamide

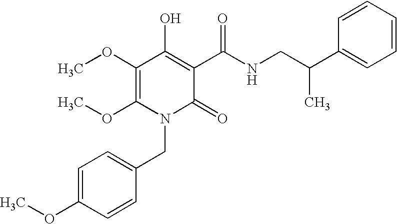

At −30° C., 22.1 mg (0.20 mmol) of ethyl chloroformate and after a further 5 minutes 26.1 mg (0.19 mmol) of 2-phenylpropan-1-amine were added dropwise to a solution of 65 mg (0.19 mmol) of 4-hydroxy-5,6-dimethoxy-1-(4-methoxybenzyl)-2-oxo-1,2-dihydropyridine-3-carboxylic acid (I-248) and 21.6 mg (0.21 mmol) of 4-methylmorpholine in 5 ml of dry tetrahydrofuran. The reaction mixture was stirred overnight and slowly warmed to room temperature in the process. The suspension was added to ice-cold IN hydrochloric acid and extracted repeatedly with dichloromethane. The organic phase was dried, filtered and concentrated. The residue was purified by column chromatography (silica gel, mobile phase: dichloromethane/methanol 100:0 to 100:2).

Yield: 9 mg (10% of theory).

LC-MS: m/z=453 [M+H]⁺.

The compounds of the formula (I) listed in Table 1 below and the compounds of the formula (II) listed in Table 2 below can be obtained analogously to the examples above and in accordance with the general descriptions of the processes according to the invention.

TABLE 1
(I)

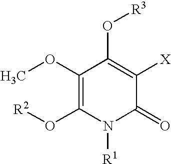

| No. | R¹ | R² | X |
|---|---|---|---|
| I-1 | H | Me | 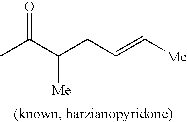 |
| I-2 | H | Me | (4-methoxyphenyl)carbonyl |
| I-3 | R-2 | H | phenylcarbonyl |
| I-4 | R-2 | H | —C(═O)tBu |
| I-5 | R-1 | H | —C(═O)Me |
| I-6 | R-2 | Me | —C(═O)CH(Me)(CH₂)₃OMe |
| I-7 | R-1 | Me | —C(═O)Me |
| I-8 | R-2 | H | —C(═O)Me |
| I-9 | R-2 | H | —C(═O)Et |
| I-10 | R-2 | H | (2-methylphenyl)carbonyl |
| I-11 | R-2 | H | (4-methylphenyl)carbonyl |
| I-12 | CH₃ | H | phenylcarbonyl |
| I-13 | H | Me | —C(═O)Me |
| I-14 | H | Me | —C(═O)Et |
| I-15 | R-3 | H | phenylcarbonyl |
| I-16 | R-4 | Me | —C(═O)CH(Me)CH₂CH═CMe₂ |
| I-17 | R-2 | Me | —C(═O)CH(Me)(CH₂)₂CMe₃ |
| I-18 | R-2 | Me | 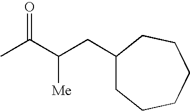 |
| I-19 | R-2 | Me | 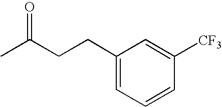 |
| I-20 | R-2 | Me | 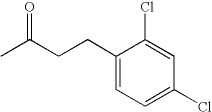 |
| I-21 | R-3 | H | —C(═O)Me |
| I-22 | H | Me | 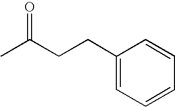 |
| I-23 | H | Me | 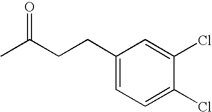 |
| I-24 | H | Me | 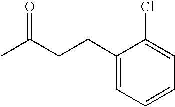 |
| I-25 | R-2 | Me | phenylcarbonyl |
| I-26 | R-2 | Me | —C(═O)Me |
| I-27 | R-2 | Me | 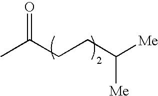 |
| I-28 | R-2 | Me | 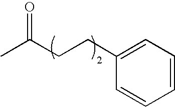 |
| I-29 | H | Me | 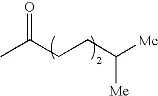 |
| I-30 | H | Me | 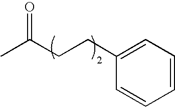 |
| I-31 | H | Me | phenylcarbonyl |
| I-32 | R-2 | Me | —C(═O)tBu |
| I-33 | R-2 | Me | (2-methylphenyl)carbonyl |
| I-34 | H | H | phenylcarbonyl |
| I-35 | R-5 | Me | —C(═O)CH(Me)(CH₂)₂CHMe₂ |
| I-36 | H | Me | —C(═O)tBu |
| I-37 | H | Me | H |
| I-38 | R-2 | Me | 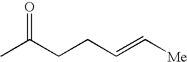 |
| I-39 | H | Me | 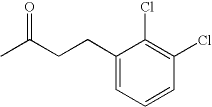 |
| I-40 | R-2 | Me | 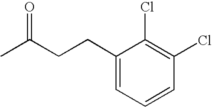 |
| I-41 | R-2 | Me | 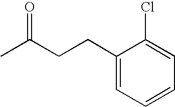 |
| I-42 | R-2 | H | —C(═O)CH(Me)(CH₂)₂CMe₃ |
| I-43 | R-2 | Me | —CO₂H |
| I-44 | R-2 | Me | 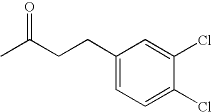 |
| I-45 | H | Me | cyclopentylcarbonyl |
| I-46 | R-2 | Me | —C(═O)CH(Me)CH₂CH═C(CH₃)Cl |
| I-47 | R-2 | Me | 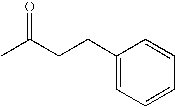 |
| I-48 | R-2 | Me | —C(═O)Et |
| I-49 | R-2 | Me | (4-methylphenyl)carbonyl |
| I-50 | R-2 | H | 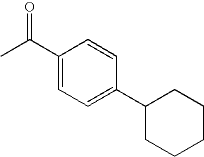 |
| I-51 | H | Me | —C(═O)CH(Me)(CH₂)₃OMe |
| I-52 | H | Me | —C(═O)CH(Et)(CH₂)₂OEt |
| I-53 | R-2 | H | benzylcarbonyl |
| I-54 | R-2 | H | —C(═O)nBu |
| I-55 | H | Me | (4-methylphenyl)carbonyl |
| I-56 | H | Me | (2-methylphenyl)carbonyl |
| I-57 | H | Me | 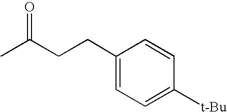 |
| I-58 | H | Me | 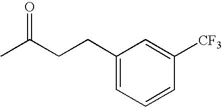 |
| I-59 | H | Me | 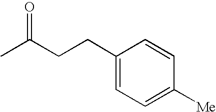 |
| I-60 | H | Me | 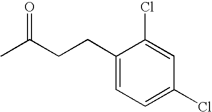 |
| I-61 | R-2 | Me | 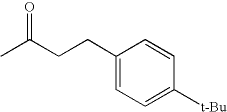 |
| I-62 | R-2 | H | 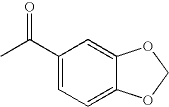 |
| I-63 | R-2 | H | (4-tert-butylphenyl)carbonyl |
| I-64 | R-1 | H | —C(═O)iPr |
| I-65 | R-2 | H | 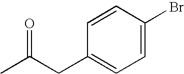 |
| I-66 | R-3 | Me | —C(═O)Me |
| I-67 | R-2 | Me | (4-chlorophenyl)carbonyl |
| I-68 | R-3 | H | cyclopropylcarbonyl |
| I-69 | R-3 | H | cyclopentylcarbonyl |
| I-70 | H | Me | 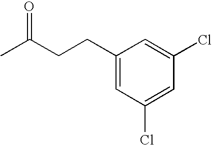 |
| I-71 | R-2 | Me | 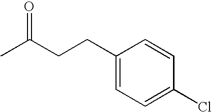 |
| I-72 | H | Me | 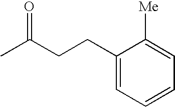 |
| I-73 | H | Me | —C(═O)CH(Et)(CH₂)₂CHMe₂ |
| I-74 | H | Me | —C(═O)CH(Me)CH₂CHEt₂ |
| I-75 | R-2 | Me | 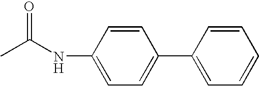 |
| I-76 | H | Me | 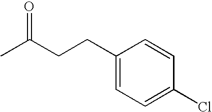 |
| I-77 | R-3 | H | 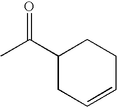 |
| I-78 | H | Me | 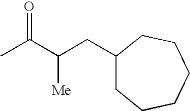 |
| I-79 | R-2 | Me | 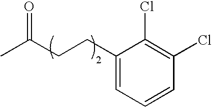 |
| I-80 | R-2 | H | 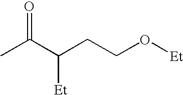 |
| I-81 | H | Me | 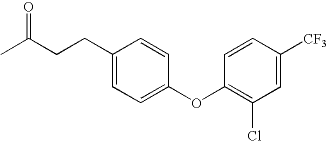 |
| I-82 | R-2 | Me | 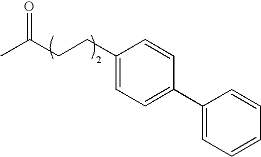 |
| I-83 | R-2 | Me | 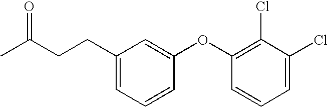 |
| I-84 | H | Me | 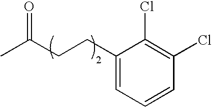 |
| I-85 | R-2 | Me | 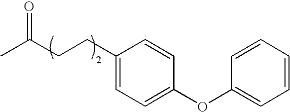 |
| I-86 | H | Me | 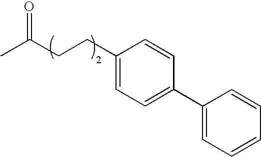 |
| I-87 | H | Me | 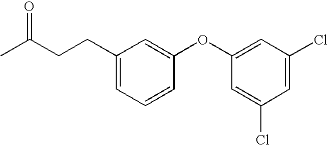 |
| I-88 | H | Me | 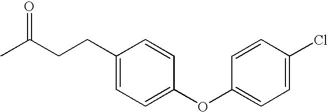 |
| I-89 | R-2 | Me | 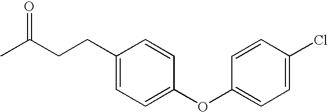 |
| I-90 | H | Me | 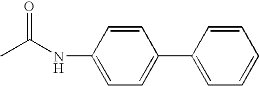 |
| I-91 | H | Me | 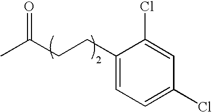 |
| I-92 | R-2 | Me | 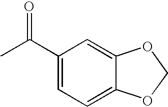 |
| I-93 | H | Me | 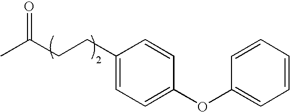 |
| I-94 | Me | H | —C(═O)Me |
| I-95 | R-2 | Me | 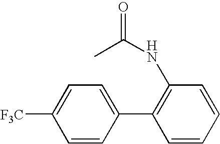 |
| I-96 | H | Me | 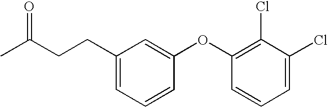 |
| I-97 | R-2 | Me | 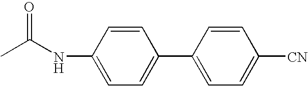 |
| I-98 | H | Me | 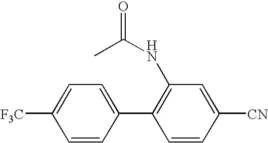 |
| I-99 | R-2 | Me | —C(═O)CH(Me)(CH₂)₂CHMe₂ |
| I-100 | R-2 | Me | —CO₂Me |
| I-101 | R-2 | H | 4-methylcyclohexylcarbonyl |
| I-102 | Me | Me | —C(═O)Me |
| I-103 | R-2 | Me | —C(═O)iPr |
| I-104 | Me | Me | 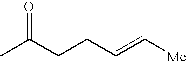 |
| I-105 | R-2 | Me | —C(═O)nBu |
| I-106 | Me | Me | phenylcarbonyl |
| I-107 | R-2 | Me | 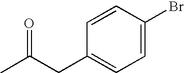 |
| I-108 | R-1 | Me | —C(═O)iPr |
| I-109 | R-3 | Me | cyclohexylcarbonyl |
| I-110 | R-3 | Me | 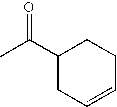 |
| I-111 | R-2 | H | cycloheptylcarbonyl |
| I-112 | R-2 | H | —C(═O)iPr |
| I-113 | H | Me | —C(═O)iPr |
| I-114 | H | Me | cyclohexylcarbonyl |
| I-115 | H | Me | 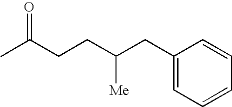 |
| I-116 | R-2 | Me | cycloheptylcarbonyl |
| I-117 | R-2 | Me | 4-methylcyclohexylcarbonyl |
| I-118 | R-3 | Me | 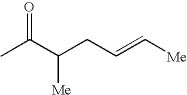 |
| I-119 | R-2 | Me | cyclopentylcarbonyl |
| I-120 | H | Me | (4-trifluoromethylphenyl)carbonyl |
| I-121 | H | Me | 4-methylcyclohexylcarbonyl |
| I-122 | H | Me | (4-chlorophenyl)carbonyl |
| I-123 | R-2 | Et | 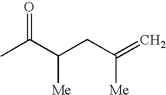 |
| I-124 | R-2 | Et | 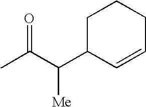 |
| I-125 | H | Me | cycloheptylcarbonyl |
| I-126 | H | Me | 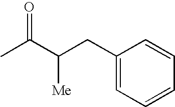 |
| I-127 | H | Me | —C(═O)nBu |
| I-128 | R-2 | Me | 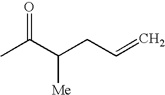 |
| I-129 | H | Me | 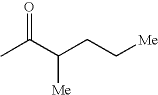 |
| I-130 | H | Me | cyclopropylcarbonyl |
| I-131 | H | Me | 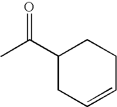 |
| I-132 | R-2 | H | 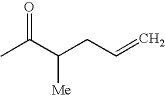 |
| I-133 | R-3 | H | 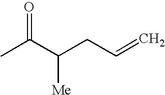 |
| I-134 | R-2 | Me | 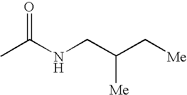 |
| I-135 | R-2 | Me | 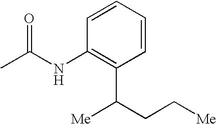 |
| I-136 | R-3 | Me | 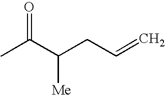 |
| I-137 | R-3 | Me | —C(═O)Et |
| I-138 | H | Me | 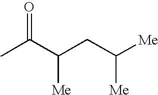 |
| I-139 | H | Me | 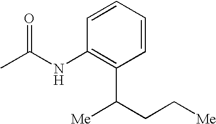 |
| I-140 | R-2 | Me | 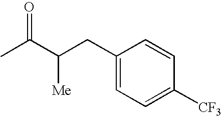 |
| I-141 | H | Me | 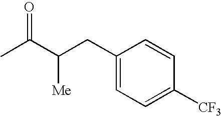 |
| I-142 | R-3 | Et | 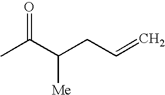 |
| I-143 | R-2 | Me | 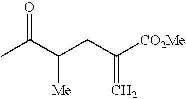 |
| I-144 | H | Me | 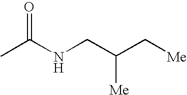 |
| I-145 | H | Et | —C(═O)CH(Me)(CH₂)₂Me |
| I-146 | H | Me | 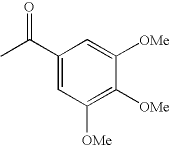 |
| I-147 | H | Me | 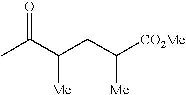 |
| I-148 | H | Me | 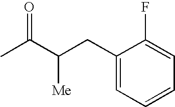 |
| I-149 | H | Me | 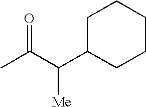 |
| I-150 | R-2 | Me | 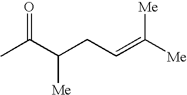 |
| I-151 | R-2 | Me | 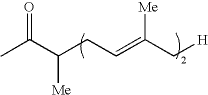 |
| I-152 | R-2 | H | —C(═O)nPr |
| I-153 | H | Me | 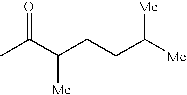 |
| I-154 | H | Me | 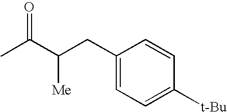 |
| I-155 | H | Me | 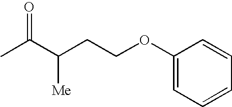 |
| I-156 | H | Me | 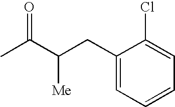 |
| I-157 | R-2 | Me | 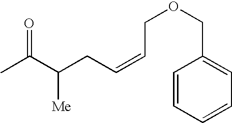 |
| I-158 | H | Me | 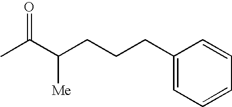 |
| I-159 | H | Me | 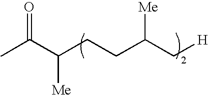 |
| I-160 | H | Me | 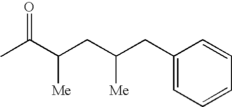 |
| I-161 | H | Me | 2-fluorophenylcarbonyl |
| I-162 | R-4 | Me | —C(═O)Et |
| I-163 | H | Me | 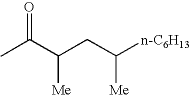 |
| I-164 | H | Me | 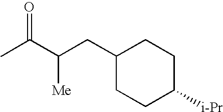 |
| I-165 | H | Me | —CO₂Me |
| I-166 | R-2 | Me | —C(═O)nPr |
| I-167 | R-2 | H | 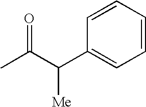 |
| I-168 | H | Me | 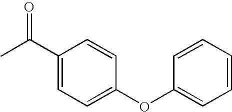 |
| I-169 | R-2 | Me | 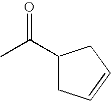 |
| I-170 | R-2 | Me | —C(═O)CH(Me)CH₂CHEt₂ |
| I-171 | H | Me | 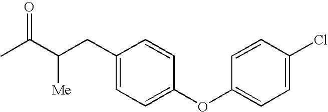 |
| I-172 | R-2 | Me | 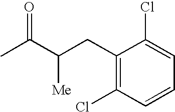 |
| I-173 | R-2 | Me | 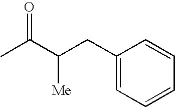 |
| I-174 | H | Me | 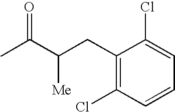 |
| I-175 | R-2 | H | 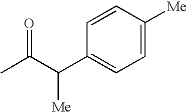 |
| I-176 | R-2 | H | 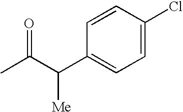 |
| I-177 | R-2 | H | —C(═O)CH(Me)(CH₂)₃Me |
| I-178 | R-2 | H | —C(═O)CH(Me)(CH₂)₄Me |
| I-179 | R-2 | H | —C(═O)CH(Et)(CH₂)₃Me |
| I-180 | R-2 | H | —C(═O)CH(Me)CH₂CF₃ |
| I-181 | R-2 | Me | 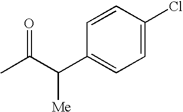 |
| I-182 | R-2 | Et | 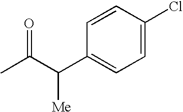 |
| I-183 | R-2 | Me | 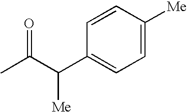 |
| I-184 | R-2 | Me | 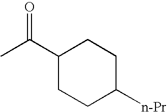 |
| I-185 | R-2 | Me | —C(═O)CH(Me)CH₂CF₃ |
| I-186 | R-2 | Me | —C(═O)CH(Me)(CH₂)₄Me |
| I-187 | R-2 | Me | —C(═O)CH(Et)(CH₂)₃Me |
| I-188 | R-2 | Me | 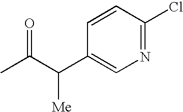 |
| I-189 | R-2 | Me | 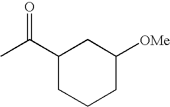 |
| I-190 | R-2 | Me | 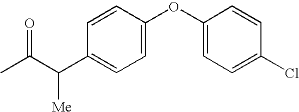 |
| I-191 | R-2 | Me | 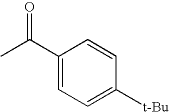 |
| I-192 | R-2 | Me | 3-methylcyclohexylcarbonyl |
| I-193 | R-2 | Me | —C(═O)CH(Me)(CH₂)₃Me |
| I-194 | R-2 | H | 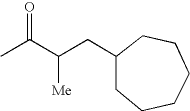 |
| I-195 | H | Me | 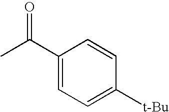 |
| I-196 | H | Me | 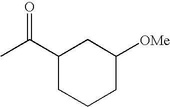 |
| I-197 | H | Me | —C(═O)CH(Me)CH₂CF₃ |
| I-198 | H | Me | —C(═O)CH(Me)(CH₂)₄Me |
| I-199 | H | Me | 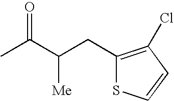 |
| I-200 | H | Me | 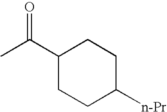 |
| I-201 | H | Me | 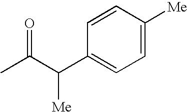 |
| I-202 | R-2 | H | 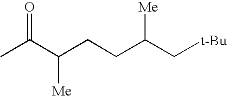 |
| I-203 | H | Me | 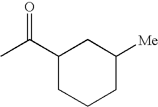 |
| I-204 | H | Me | 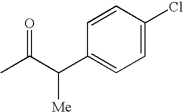 |
| I-205 | H | Et | 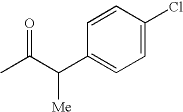 |
| I-206 | H | Me | 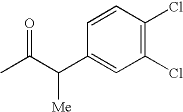 |
| I-207 | H | Me | 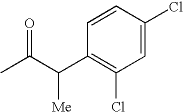 |
| I-208 | H | Me | —C(═O)CH(Et)(CH₂)₃Me |
| I-209 | H | Me | 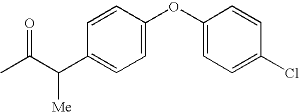 |
| I-210 | H | Me | 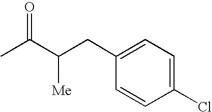 |
| I-211 | H | Me | —C(═O)CH-nPr₂ |
| I-212 | H | Me | 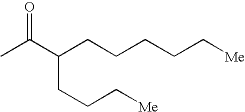 |
| I-213 | H | Et | 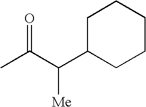 |
| I-214 | H | Me | 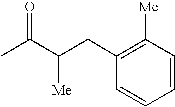 |
| I-215 | H | Me | 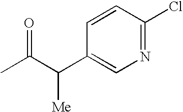 |
| I-216 | H | Me | 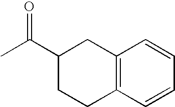 |
| I-217 | H | Me | 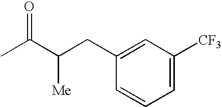 |
| I-218 | H | Me | 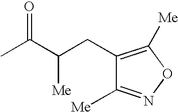 |
| I-219 | H | Me | 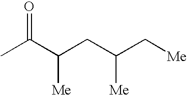 |
| I-220 | H | Me | 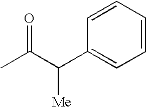 |
| I-221 | H | Me | 2-naphthylcarbonyl |
| I-222 | H | Et | 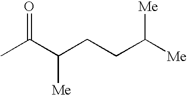 |
| I-223 | R-2 | Me | 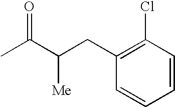 |
| I-224 | R-2 | H | —C(═O)CH(Me)(CH₂)₃OMe |
| I-225 | R-2 | H | 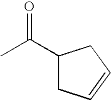 |
| I-226 | R-2 | Me | 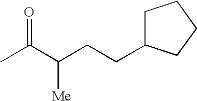 |
| I-227 | H | Me | 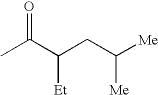 |
| I-228 | H | Me | —C(═O)CH(Me)(CH₂)₂CMe₃ |
| I-229 | R-2 | H | 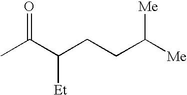 |
| I-230 | H | Me | —C(═O)CH(Me)(CH₂)₃Me |
| I-231 | R-1 | H | phenylcarbonyl |
| I-232 | R-1 | H | (4-tert-butylphenyl)carbonyl |
| I-233 | R-1 | H | (4-methoxyphenyl)carbonyl |
| I-234 | R-2 | H | (4-methoxyphenyl)carbonyl |
| I-235 | R-2 | Me | (4-methoxyphenyl)carbonyl |
| I-236 | R-1 | H | 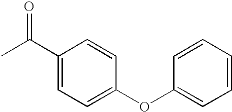 |
| I-237 | R-1 | Me | phenylcarbonyl |
| I-238 | R-2 | H | —CO₂Me |
| I-239 | H | Me | 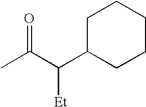 |
| I-240 | H | Me | 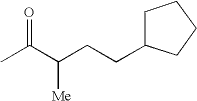 |
| I-241 | R-2 | Me | —C(═O)CH(Et)(CH₂)₂CH(CH₃)₂ |
| I-242 | R-2 | Me | 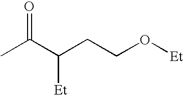 |
| I-243 | H | Me | 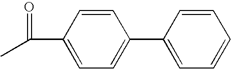 |
| I-244 | R-2 | Me | 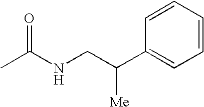 |
| I-245 | H | H | —CO₂Me |
| I-246 | R-2 | Me | 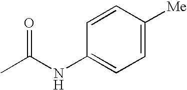 |
| I-247 | R-2 | H | 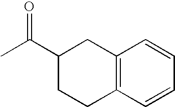 |
| I-248 | H | Me | 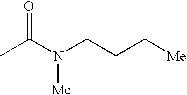 |
| I-249 | R-2 | H | (4-chlorophenyl)carbonyl |
| I-250 | R-2 | H | —C(═O)CH(Me)CH₂CHEt₂ |
| I-251 | R-2 | H | 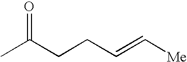 |
| I-252 | R-3 | H | cyclohexylcarbonyl |
| I-253 | H | Me | 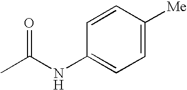 |
| I-254 | Me | H | 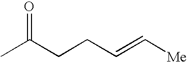 |
| I-255 | R-2 | Me | H |
| I-256 | R-3 | H | 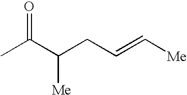 |
| I-257 | R-2 | Me | 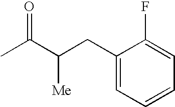 |
| I-258 | R-2 | Me | 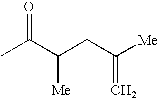 |
| I-259 | R-2 | Me | 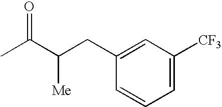 |
| I-260 | R-3 | Me | 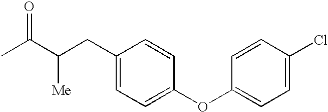 |
| I-261 | R-2 | Et | —C(═O)Et |
| I-262 | R-2 | Me | 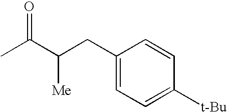 |
| I-263 | R-2 | H | 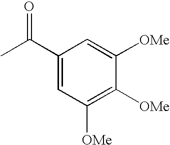 |
| I-264 | R-2 | H | 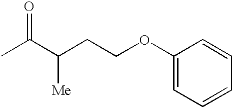 |
| I-265 | R-2 | Me | 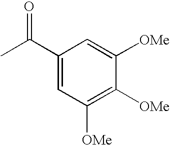 |
| I-266 | R-2 | Me | 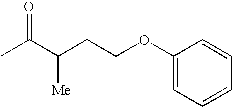 |
| I-267 | R-2 | Me | 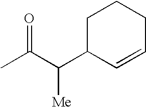 |
| I-268 | R-2 | Me | 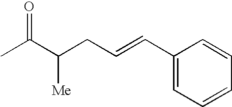 |
| I-269 | R-2 | Me | 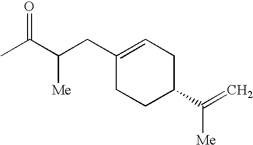 |
| I-270 | R-2 | Me | 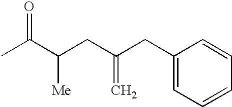 |
| I-271 | R-2 | Me | 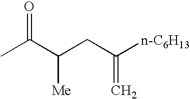 |
| I-272 | R-4 | H | —C(═O)Et |
| I-273 | R-2 | Me | 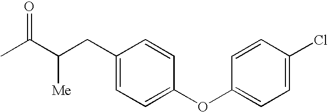 |
| I-274 | R-2 | H | 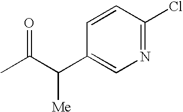 |
| I-275 | R-2 | Me | 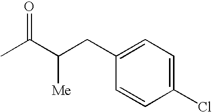 |
| I-276 | R-2 | Me | 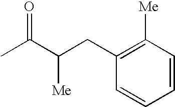 |
| I-277 | R-2 | Me | 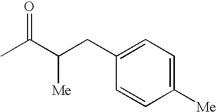 |
| I-278 | H | Me | 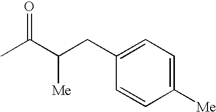 |
| I-279 | R-2 | H | 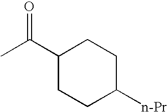 |
| I-280 | R-2 | H | —C(═O)CH(Me)(CH₂)₂CHMe₂ |
| I-281 | R-2 | H | —C(═O)CH-nPr₂ |
| I-282 | R-2 | H | 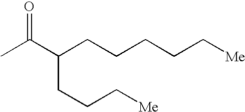 |
| I-283 | R-2 | H | 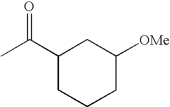 |
| I-284 | R-2 | H | 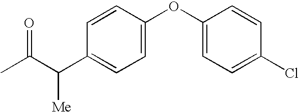 |
| I-285 | R-2 | H | 3-methylcyclohexylcarbonyl |
| I-286 | R-2 | Me | 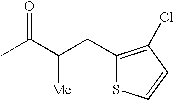 |
| I-287 | R-2 | Me | —C(═O)CH-nPr₂ |
| I-288 | R-2 | Me | 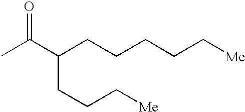 |
| I-289 | R-2 | H | 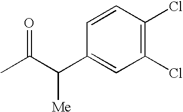 |
| I-290 | R-2 | H | 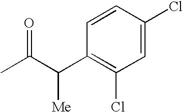 |
| I-291 | R-2 | Me | 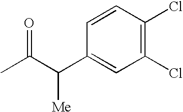 |
| I-292 | R-2 | Me | 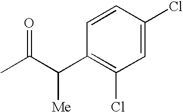 |
| I-293 | R-2 | H | 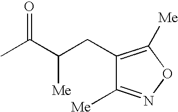 |
| I-294 | R-2 | Me | 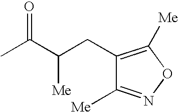 |
| I-295 | R-2 | Me | 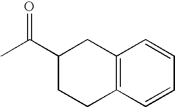 |
| I-296 | R-2 | H | 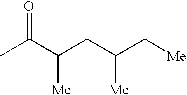 |
| I-297 | R-2 | H | 2-naphthylcarbonyl |
| I-298 | R-2 | H | 2-fluorophenylcarbonyl |
| I-299 | R-2 | H | 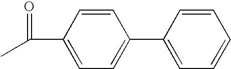 |
| I-300 | R-2 | H | (4-trifluoromethylphenyl)carbonyl |
| I-301 | R-2 | Et | 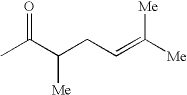 |
| I-302 | R-2 | Me | 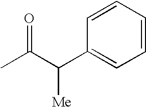 |
| I-303 | R-2 | Me | 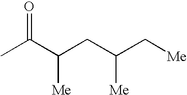 |
| I-304 | R-2 | H | —C(═O)CH(Me)CH₂CH═C(CH₃)Cl |
| I-305 | R-2 | Me | (4-trifluoromethylphenyl)carbonyl |
| I-306 | R-2 | Me | 2-naphthylcarbonyl |
| I-307 | R-2 | Me | 2-fluorophenylcarbonyl |
| I-308 | R-2 | Me | 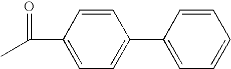 |
| I-309 | H | Et | 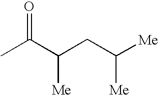 |
| I-310 | R-2 | H | 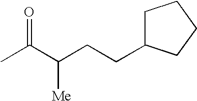 |
| I-311 | R-2 | H | cyclopentylcarbonyl |
| I-312 | R-2 | Me | 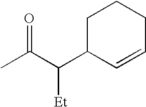 |
| I-313 | R-2 | Me | 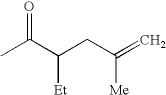 |
| I-314 | R-2 | Me | 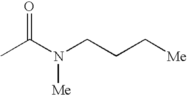 |
| I-315 | R-1 | Me | 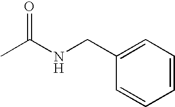 |

R³ = H
Me = methyl, Et = ethyl, nPr = n-propyl, iPr = isopropyl, nBu = n-butyl,
tBu = tert-butyl

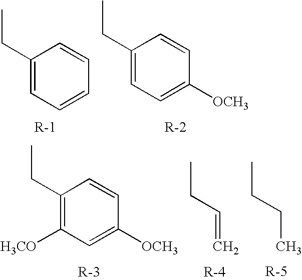

TABLE 2
(II)

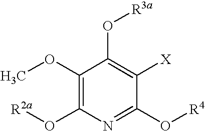

| No. | R^{2a} | R^{3a} | R⁴ | X | logP (pH 2.3) |
|---|---|---|---|---|---|
| II-1 | Me | C(═O)Me | C(═O)Me | —C(═O)CH(CH₃)(CH₂)₃OCH₃ | 2.79 |

TABLE 3

| No. | Physical data^{a)} |
|---|---|
| I-1 | δ (DMSO-d₆) = 1.05 (d, 3H), 1.59 (d, 3H), 1.96 (m, 1H), 2.35 (m, 1H), 3.61 (s, 3H), |
| | 3.84 (m, 1H), 3.92 (s, 3H), 5.34-5.43 (m, 2H); LC-MS: m/z = 282 [M + H]⁺; known |
| I-2 | δ = 3.64 (s, 3H), 3.67 (s, 3H), 3.83 (s, 3H), 6.82 (d, 2H), 7.63 (d, 2H); LC-MS: m/z = 306 [M + H]⁺ |
| I-3 | δ = 3.78 (s, 3H), 3.84 (s, 3H), 5.17 (s, 2H), 6.34 (s, 1H), 6.85 (d, 2H), 7.43-7.53 (m, 7H), |
| | LC-MS: m/z = 382 [M + H]⁺ |
| I-4 | δ = 1.39 (s, 9H), 3.78 (s, 3H), 3.91 (s, 3H), 5.17 (s, 2H), 6.83 (d, 2H), 7.46 (d, 2H); LC-MS: |
| | m/z = 362 [M + H]⁺ |
| I-5 | δ (DMSO-d₆) = 2.38 (s, 3H), 3.48 (s, 3H), 4.95 (s, 2H), 7.11-7.29 (m, 5H); LC-MS: m/z = 290 [M + H]⁺ |
| I-6 | δ (DMSO-d₆) = 1.07 (d, 3H), 1.36 (m, 1H), 1.49 (m, 2H), 1.70 (m, 1H), 3.18 (s, 3H), 3.28 (t, |
| | 2H), 3.69 (s, 3H), 3.72 (s, 3H), 4.12 (s, 3H), 4.13 (m, 1H), 5.05 (s, 2H), 6.87 (d, 2H), |
| | 7.19 (d, 2H); LC-MS: m/z = 338 [M + H]⁺ |
| I-7 | δ = 2.65 (s, 3H), 3.70 (s, 3H), 4.02 (s, 3H), 5.13 (s, 2H), 7.15-7.21 (m, 5H); LC-MS: m/z = 304 [M + H]⁺ |
| I-8 | δ = 2.60 (s, 3H), 3.77 (s, 3H), 3.89 (s, 3H), 5.11 (s, 2H), 6.82 (d, 2H), 7.43 (d, 2H); LC-MS: |
| | m/z = 320 [M + H]⁺ |
| I-9 | δ^{b)} = 1.22 (t, 3H), 3.01 (q, 2H), 3.78 (s, 3H), 3.89 (s, 3H), 5.12 (s, 2H), 6.82 (d, 2H), 7.43 (d, |
| | 2H); LC-MS: m/z = 334 [M + H]⁺ |
| I-10 | δ^{b)} = 2.30 (s, 3H), 3.79 (s, 3H), 3.91 (s, 3H), 5.17 (s, 2H), 6.18 (s, 1H), 6.88 (d, 2H), |
| | 7.16-7.25 (m, 3H), 7.34 (t, 1H), 7.50 (d, 2H); LC-MS: m/z = 396 [M + H]⁺ |
| I-11 | δ^{b)} = 2.42 (s, 3H), 3.78 (s, 3H), 3.85 (s, 3H), 5.18 (s, 2H), 6.35 (s, 1H), 6.85 (d, 2H), |
| | 7.22 (d, 2H), 7.43 (d, 2H), 7.48 (d, 2H); LC-MS: m/z = 396 [M + H]⁺ |
| I-12 | δ = 3.44 (s, 3H), 3.84 (s, 3H), 6.39 (s, 1H), 7.43 (t, 2H), 7.51-7.56 (m, 3H); LC-MS: m/z = 276 [M + H]⁺ |
| I-13 | δ = 2.77 (s, 3H), 3.76 (s, 3H), 4.22 (s, 3H); LC-MS: m/z = 214 [M + H]⁺ |
| I-14 | δ = 1.14 (t, 3H), 3.10 (q, 2H), 3.73 (s, 3H), 4.01 (s, 3H); LC-MS: m/z = 228 [M + H]⁺ |
| I-15 | δ = 3.78 (s, 3H), 3.86 (s, 6H), 5.22 (s, 2H), 6.38-6.47 (m, 3H), 6.94 (d, 1H), 7.43 (t, 2H), |
| | 7.50-7.61 (m, 3H); LC-MS: m/z = 410 [M − H]⁻ |
| I-16 | δ (DMSO-d₆) = 1.04 (d, 3H), 1.55 (s, 3H), 1.64 (s, 3H), 2.04 (m, 1H), 2.34 (m, 1H), 3.71 (s, |
| | 3H), 4.10 (m, 1H), 4.19 (s, 3H), 4.53 (d, 2H), 5.02-5.13 (m, 3H), 5.86 (m, 1H); LC-MS: m/z = 336 [M + H]⁺ |
| I-17 | δ (DMSO-d₆) = 0.83 (s, 9H), 1.07 (d, 3H), 1.13-1.21 (m, 2H), 1.25 (m, 1H), 1.65 (m, 1H), |
| | 3.69 (s, 3H), 3.72 (s, 3H), 4.05 (m, 1H), 4.12 (s, 3H), 5.03-5.07 (m, 2H), 6.86 (d, 2H), |
| | 7.19 (d, 2H); LC-MS: m/z = 432 [M + H]⁺ |
| I-18 | δ (DMSO-d₆) = 1.05 (d, 3H), 1.10-1.20 (m, 2H), 1.30-1.67 (m, 13H), 3.69 (s, 3H), |
| | 3.72 (s, 3H), 4.12 (s, 3H), 4.31 (m, 1H), 5.04-5.07 (m, 2H), 6.86 (d, 2H), 7.19 (d, 2H); LC-MS: |
| | m/z = 458 [M + H]⁺ |
| I-19 | δ = 3.05 (t, 2H), 3.52 (t, 2H), 3.76 (s, 3H), 3.78 (s, 3H), 4.14 (s, 3H), 5.12 (s, 2H), 6.84 (d, |
| | 2H), 7.23 (d, 2H), 7.37-7.49 (m, 3H), 7.53 (s, 1H); LC-MS: m/z = 492 [M + H]⁺ |
| I-20 | δ = 3.08 (t, 2H), 3.49 (t, 2H), 3.76 (s, 3H), 3.78 (s, 3H), 4.13 (s, 3H), 5.12 (s, 2H), 6.83 (d, |
| | 2H), 7.13-7.36 (m, 5H); LC-MS: m/z = 492 [M(³⁵Cl) + H⁺, 2 Cl] |
| I-21 | δ = 2.62 (s, 3H), 3.76 (s, 3H), 3.84 (s, 3H), 3.89 (s, 3H), 5.16 (s, 2H), 6.38 (dd, 1H), 6.45 (d |
| | (1H), 6.83 (d, 1H); LC-MS: m/z = 348 [M − H]⁺ |
| I-22 | δ = 3.00 (t, 2H), 3.40 (t, 2H), 3.80 (s, 3H), 4.03 (s, 3H), 7.20 (t, 1H), 7.22-7.32 (m, 4H); |
| | LC-MS: m/z = 304 [M + H]⁺ |
| I-23 | δ = 2.98 (t, 2H), 3.38 (t, 2H), 3.83 (s, 3H), 4.10 (s, 3H), 7.10 (d, 1H), 7.26 (s, 1H), 7.35 (m, |
| | 1H); LC-MS: m/z = 372 [M(Cl³⁵) + H⁺, 2 Cl] |
| I-24 | δ = 3.11 (t, 2H), 3.38 (t, 2H), 3.77 (s, 3H), 3.95 (s, 3H), 7.13-7.22 (m, 2H), 7.30 (m, 1H), |
| | 7.35 (m, 1H); LC-MS: m/z = 338 [M(Cl³⁵) + H⁺, 1 Cl] |
| I-25 | δ = 3.78 (s, 3H), 3.81 (s, 3H), 4.14 (s, 3H), 5.07 (s, 2H), 6.81 (d, 2H), 7.21 (d, 2H), 7.41 (t, |
| | 2H), 7.48 (t, 1H), 7.58 (d, 2H), LC-MS: m/z = 396 [M + H]⁺ |
| I-26 | δ = 2.72 (s, 3H), 3.75 (s, 3H), 3.78 (s, 3H), 4.13 (s, 3H), 5.11 (s, 2H), 6.83 (d, 2H), 7.25 (d, |
| | 2H); LC-MS: m/z = 334 [M + H]⁺ |
| I-27 | δ = 0.87 (d, 6H), 1.21 (m, 2H), 1.36 (m, 2H), 1.50-1.75 (m, 3H), 3.20 (t, 2H), 3.75 (s, 3H), |
| | 3.78 (s, 3H), 4.11 (s, 3H), 5.12 (s, 2H), 6.83 (d, 2H), 7.25 (d, 2H); LC-MS: m/z = 418 [M + H]⁺ |
| I-28 | δ = 1.70-1.80 (m, 4H), 2.70 (t, 2H), 3.21 (t, 2H), 3.75 (s, 3H), 3.78 (s, 3H), 4.11 (s, 3H), |
| | 5.11 (s, 2H), 6.83 (d, 2H), 7.12-7.33 (d, 7H); LC-MS: m/z = 452 [M + H]⁺ |
| I-29 | δ = 0.89 (d, 6H), 1.20 (m, 2H), 1.35 (m, 2H), 1.55 (m, 1H), 1.65 (m, 2H), 3.05 (t, 2H), |
| | 3.82 (s, 3H), 4.14 (s, 3H); LC-MS: m/z = 298 [M + H]⁺ |
| I-30 | δ = 1.65-1.80 (m, 4H), 2.65 (t, 2H), 3.09 (t, 2H), 3.79 (s, 3H), 4.11 (s, 3H), 7.15-7.22 (m, |
| | 3H), 7.26 (t, 2H); LC-MS: m/z = 332 [M + H]⁺ |
| I-31 | δ = 3.72 (s, 3H), 3.79 (s, 3H), 7.39 (t, 2H), 7.48 (t, 1H), 7.55 (d, 2H); LC-MS: m/z = 276 [M + H]⁺ |
| I-32 | δ = 1.43 (s, 9H), 3.75 (s, 3H), 3.78 (s, 3H), 4.11 (s, 3H), 5.15 (s, 2H), 6.83 (d, 2H), 7.26 (d, |
| | 2H); LC-MS: m/z = 376 [M + H]⁺ |
| I-33 | δ = 2.30 (s, 3H), 3.79 (s, 3H), 3.81 (s, 3H), 4.11 (s, 3H), 5.03 (s, 2H), 6.83 (d, 2H), |
| | 7.15-7.35 (m, 6H); LC-MS: m/z = 410 [M + H]⁺ |
| I-34 | δ (DMSO-d₆) = 3.55 (s, 3H), 7.38-7.50 (m, 5H); LC-MS: m/z = 262 [M + H]⁺ |
| I-35 | δ (DMSO-d₆) = 0.82-0.86 (m, 9H), 1.05 (d, 3H), 1.12-1.18 (m, 2H), 1.25-1.70 (m, 7H), |
| | 3.70 (s, 3H), 3.87 (t, 2H), 4.09 (m, 1H), 4.23 (s, 3H); LC-MS: m/z = 340 [M + H]⁺ |
| I-36 | δ = 1.38 (s, 9H), 3.79 (s, 3H), 4.14 (s, 3H); LC-MS: m/z = 256 [M + H]⁺ |
| I-37 | δ = 3.73 (s, 3H), 3.90 (s, 3H), 5.84 (s, 1H); LC-MS: m/z = 172 [M + H]⁺ |
| I-38 | δ = 1.64 (d, 3H), 2.36 (m, 2H), 3.25 (t, 2H), 3.76 (s, 3H), 3.79 (s, 3H), 4.12 (s, 3H), 5.13 (s, |
| | 2H), 5.47-5.53 (m 2H), 6.84 (d, 2H), 7.24 (d, 2H); LC-MS: m/z = 388 [M + H]⁺ |
| I-39 | δ = 3.14 (t, 2H), 3.41 (t, 2H), 3.76 (s, 3H), 4.02 (s, 3H), 7.15 (t, 1H), 7.24 (d, 1H), 7.35 (m, |
| | 1H); LC-MS: m/z = 372 [M(Cl³⁵) + H⁺, 2 Cl] |
| I-40 | δ = 3.14 (t, 2H), 3.53 (t, 2H), 3.76 (s, 3H), 3.78 (s, 3H), 4.14 (s, 3H), 5.11 (s, 2H), 6.83 (d, |
| | 2H), 7.10 (t, 1H), 7.21-7.35 (m, 4H); LC-MS: m/z = 492 {M(Cl³⁵) + H]⁺, 2 Cl} |
| I-41 | δ = 3.12 (t, 2H), 3.51 (t, 2H), 3.74 (s, 3H), 3.77 (s, 3H), 4.14 (s, 3H), 5.12 (s, 2H), 6.83 (d, |
| | 2H), 7.11-7.38 (m, 6H); LC-MS: m/z = 458 {[M(Cl³⁵) + H]⁺, 1 Cl} |
| I-42 | log P (HCO₂H) = 4.72; LC-MS: m/z = 418 [M + H]^{+c)} |
| I-43 | δ (DMSO-d₆) = 3.73 (s, 3H), 3.75 (s, 3H), 4.16 (s, 3H), 5.13 (s, 2H), 6.89 (d, 2H), 7.23 (d, |
| | 2H); LC-MS: m/z = 336 [M + H]⁺ |
| I-44 | δ = 2.95 (t, 2H), 3.50 (t, 2H), 3.76 (s, 3H), 3.79 (s, 3H), 4.12 (s, 3H), 5.12 (s, 2H), 6.84 (d, |
| | 2H), 7.12 (m, 1H), 7.19-7.37 (m, 4H); LC-MS: m/z = 492 {[M(Cl³⁵) + H]⁺, 2 Cl} |
| I-45 | δ (DMSO-d₆) = 1.52-1.70 (m, 4H), 1.71-1.80 (m, 2H), 1.82-1.91 (m, 2H), 3.11 (s, 3H), |
| | 3.62 (s, 3H), 4.05 (m, 1H); LC-MS: m/z = 268 [M + H]⁺ |
| I-46 | δ (DMSO-d₆) = 1.08 (d, 3H), 2.05 (s, 3H), 2.28 (m, 1H), 2.41 (m, 1H), 3.69 (s, 3H), 3.72 (s, |
| | 3H), 4.12 (s, 3H), 4.17 (m, 1H), 5.05 (s, 2H), 5.57 (t, 1H), 6.87 (d, 2H), 7.19 (d, 2H); LC- |
| | MS: m/z = 436 {[M(Cl³⁵) + H]⁺, 1 Cl} |
| I-47 | δ = 3.00 (t, 2H), 3.52 (t, 2H), 3.76 (s, 3H), 3.78 (s, 3H), 4.12 (s, 3H), 5.12 (s, 2H), 6.83 (d, |
| | 2H), 7.19 (m, 1H), 7.22-7.27 (m, 6H); LC-MS: m/z = 424 [M + H]⁺ |
| I-48 | δ = 1.17 (t, 3H), 3.21 (q, 2H), 3.76 (s, 3H), 3.78 (s, 3H), 4.12 (s, 3H), 5.12 (s, 2H), 6.84 (d, |
| | 2H), 7.25 (d, 2H); LC-MS: m/z = 348 [M + H]⁺ |
| I-49 | δ = 2.39 (s, 3H), 3.79 (s, 3H), 3.81 (s, 3H), 4.14 (s, 3H), 5.05 (s, 2H), 6.81 (d, 2H), |
| | 7.17-7.24 (m, 4H), 7.50 (d, 2H); LC-MS: m/z = 410 [M + H]⁺ |
| I-50 | δ = 1.22-1.95 (m, 10H), 2.57 (m, 1H), 3.79 (s, 3H), 3.85 (s, 3H), 5.17 (s, 2H), 6.37 (s, 1H), |
| | 6.85 (d, 2H), 7.23-7.26 (m, 2H), 7.46-7.50 (m, 4H); LC-MS: m/z = 464 [M + H]⁺ |
| I-51 | δ (DMSO-d₆) = 1.08 (d, 3H), 1.35 (m, 1H), 1.44-1.52 (m, 2H), 1.71 (m, 1H), 3.18 (s, 3H), |
| | 3.28 (t, 2H), 3.63 (s, 3H), 3.88 (m, 1H), 3.94 (s, 3H); LC-MS: m/z = 300 [M + H]⁺ |
| I-52 | δ (DMSO-d₆) = 0.84 (t, 3H), 0.99 (t, 3H), 1.47 (m, 1H), 1.60-1.71 (m, 2H), 1.93 (m, 1H), |
| | 3.28-3.35 (m, 4H), 3.62 (s, 3H), 3.94 (s, 3H), 3.96 (m, 1H); LC-MS: m/z = 314 [M + H]⁺ |
| I-53 | δ = 3.77 (s, 3H), 3.90 (s, 3H), 4.31 (s, 2H), 5.10 (s, 2H), 6.81 (d, 2H), 6.88 (s, 1H), |
| | 7.28-7.32 (m, 5H), 7.42 (d, 2H); LC-MS: m/z = 396 [M + H]⁺ |
| I-54 | δ = 0.94 (t, 3H), 1.41 (m, 2H), 1.67 (m, 2H), 2.96 (m, 2H), 3.77 (s, 3H), 3.88 (m, 3H), |
| | 5.11 (s, 2H), 6.82 (d, 2H), 7.43 (d, 2H); LC-MS: m/z = 362 [M + H]⁺ |
| I-55 | δ = 2.39 (s, 3H), 3.67 (s, 3H), 3.78 (s, 3H), 7.19 (d, 2H), 7.47 (d, 2H); LC-MS: m/z = 290 [M + H]⁺ |
| I-56 | δ = 2.28 (s, 3H), 3.60 (s, 3H), 3.76 (s, 3H), 7.11-7.31 (m, 4H); LC-MS: m/z = 290 [M + H]⁺ |
| I-57 | δ = 1.31 (s, 9H), 2.96 (t, 2H), 3.37 (t, 2H), 3.77 (s, 3H), 3.97 (s, 3H), 7.15 (d, 2H), 7.32 (d, |
| | 2H); LC-MS: m/z = 360 [M + H]⁺ |
| I-58 | δ (DMSO-d₆) = 2.99 (t, 2H), 3.36 (t, 2H), 3.61 (s, 3H), 3.93 (s, 3H), 7.50-7.60 (m, 3H), |
| | 7.63 (s, 1H); LC-MS: m/z = 372 [M + H]⁺ |
| I-59 | δ (DMSO-d₆) = 2.26 (s, 3H), 2.84 (t, 2H), 3.29 (t, 2H), 3.61 (s, 3H), 3.92 (s, 3H), 7.08 (d, |
| | 2H), 7.13 (d, 2H); LC-MS: m/z = 318 [M + H]⁺ |
| I-60 | δ (DMSO-d₆) = 3.01 (t, 2H), 3.34 (t, 2H), 3.63 (s, 3H), 3.94 (s, 3H), 7.33 (dd, 1H), 7.38 (d, |
| | 1H), 7.52 (d, 1H); LC-MS: m/z = 372 {[M(Cl³⁵) + H]⁺, 2 Cl} |
| I-61 | δ = 1.30 (s, 9H), 2.97 (t, 2H), 3.51 (t, 2H), 3.76 (s, 3H), 3.78 (s, 3H), 4.12 (s, 3H), 5.12 (s, |
| | 2H), 6.83 (d, 2H), 7.20-7.25 (m, 4H), 7.30 (d, 2H); LC-MS: m/z = 480 [M + H]⁺ |
| I-62 | δ = 3.78 (s, 3H), 3.86 (s, 3H), 5.16 (s, 2H), 6.03 (s, 2H), 6.50 (br. s, 1H), 6.82-6.86 (m, |
| | 3H), 7.02 (d, 1H), 7.12 (dd, 1H), 7.47 (d, 2H); LC-MS: m/z = 426 [M + H]⁺ |
| I-63 | δ = 1.34 (s, 9H), 3.79 (s, 3H), 3.84 (s, 3H), 5.17 (s, 2H), 6.37 (s, 1H), 6.85 (d, 2H), |
| | 7.42-7.50 (m, 6H); LC-MS: m/z = 438 [M + H]⁺ |
| I-64 | δ = 1.21 (d, 6H), 3.84 (m, 1H), 3.90 (s, 3H), 5.21 (s, 2H), 7.28-7.35 (m, 3H), 7.48 (d, 2H); |
| | LC-MS: m/z = 318 [M + H]⁺ |
| I-65 | δ = 3.77 (s, 3H), 3.90 (s, 3H), 4.25 (s, 2H), 5.10 (s, 2H), 6.81 (d, 2H), 7.19 (d, 2H), |
| | 7.40-7.44 (m, 4H); LC-MS: m/z = 474 {[M(Br⁷⁹) + H]⁺, 1 Br} |
| I-66 | δ = 2.70 (s, 3H), 3.75 (s, 3H), 3.82 (s, 3H), 3.91 (s, 3H), 4.05 (s, 3H), 5.15 (s, 2H), 6.37 (dd, |
| | 1H), 6.43 (d, 1H), 6.73 (d, 1H); LC-MS: m/z = 362 [M − H]⁻ |
| I-67 | δ = 3.78 (s, 3H), 3.81 (s, 3H), 4.14 (s, 3H), 5.05 (s, 2H), 6.82 (d, 2H), 7.18 (d, 2H), 7.37 (d, |
| | 2H), 7.52 (d, 2H); LC-MS: m/z = 430 {[M(Cl³⁵) + H]⁺, 1 Cl} |
| I-68 | δ = 1.11-1.15 (m, 2H), 1.35-1.38 (m, 2H), 3.19 (m, 1H), 3.76 (s, 3H), 3.83 (s, 3H), |
| | 3.91 (s, 3H), 5.17 (s, 2H), 6.37 (dd, 1H), 6.44 (d, 1H), 6.81 (d, 1H); LC-MS: m/z = 374 [M − H]⁻ |
| I-69 | δ = 1.55-2.00 (m, 8H), 3.76 (s, 3H), 3.80 (s, 3H), 3.91 (s, 3H), 4.01 (m, 1H), 5.17 (s, 2H), |
| | 6.38 (dd, 1H), 6.45 (d, 1H), 6.84 (d, 1H), 7.86 (br, s, 1H); LC-MS: m/z = 402 [M − H]⁻ |
| I-70 | δ = 2.97 (t, 2H), 3.38 (t, 2H), 3.82 (s, 3H), 4.10 (s, 3H), 7.16 (s, 2H), 7.20 (s, 1H); LC-MS: |
| | m/z = 372 {[M(Cl³⁵) + H]⁺, 2 Cl} |
| I-71 | δ = 2.97 (t, 2H), 3.49 (t, 2H), 3.76 (s, 3H), 3.78 (s, 3H), 4.13 (s, 3H), 5.12 (s, 2H), 6.83 (d, |
| | 2H), 7.21-7.28 (m, 6H); LC-MS: m/z = 458 {[M(Cl³⁵) + H]⁺, 1 Cl} |
| I-72 | δ = 2.34 (s, 3H), 2.97 (t, 2H), 3.34 (t, 2H), 3.80 (s, 3H), 3.99 (s, 3H), 7.10-7.18 (m, 4H); |
| | LC-MS: m/z = 318 [M + H]⁺ |
| I-73 | δ (DMSO-d₆) = 0.81-0.87 (m, 9H), 1.09-1.16 (m, 2H), 1.35-1.50 (m, 3H), |
| | 1.62-1.71 (m, 2H), 3.62 (s, 3H), 3.87 (m, 1H), 3.93 (s, 3H); LC-MS: m/z = 312 [M + H]⁺ |
| I-74 | δ (DMSO-d₆) = 0.76-0.83 (m, 6H), 1.06 (d, 3H), 1.15-1.31 (m, 6H), 1.70 (m, 1H), |
| | 3.62 (s, 3H), 3.93 (s, 3H), 4.07 (m, 1H); LC-MS: m/z = 312 [M + H]⁺ |
| I-75 | δ = 3.79 (s, 3H), 3.81 (s, 3H), 4.10 (s, 3H), 5.22 (s, 2H), 6.86 (d, 2H), 7.28 (d, 2H), 7.33 (t, |
| | 1H), 7.44 (t, 2H), 7.59 (m, 4H), 7.73 (d, 2H); LC-MS: m/z = 487 [M + H]⁺ |
| I-76 | δ (DMSO-d₆) = 2.90 (t, 2H), 3.33 (t, 2H), 3.63 (s, 3H), 3.94 (s, 3H), 7.25-7.32 (m, 4H); |
| | LC-MS: m/z = 338 {[M(Cl³⁵) + H]⁺, 1 Cl} |
| I-77 | δ = 1.72-2.41 (m, 6H), 3.76-3.85 (m, 1H), 3.77 (s, 3H), 3.84 (s, 3H), 3.91 (s, 3H), |
| | 5.17 (s, 2H), 5.68-5.78 (m, 2H), 6.38 (dd, 1H), 6.45 (d, 1H), 6.85 (d, 1H); LC-MS: m/z = 414 [M − H]⁻ |
| I-78 | δ (DMSO-d₆) = 1.06 (d, 3H), 1.10-1.21 (m, 2H), 1.31-1.72 (m, 13H), 3.62 (s, 3H), |
| | 3.93 (s, 3H), 4.02 (m, 1H); LC-MS: m/z = 338 [M + H]⁺ |
| I-79 | δ = 1.67-1.78 (m, 4H), 2.81 (t, 2H), 3.23 (t, 2H), 3.76 (s, 3H), 3.79 (s, 3H), 4.14 (s, 3H), |
| | 5.11 (s, 2H), 6.83 (d, 2H), 7.08-7.31 (m, 5H); LC-MS: m/z = 520 {[M(Cl³⁵) + H]⁺, 2 Cl} |
| I-80 | δ = 0.91 (t, 3H), 1.07 (t, 3H), 1.59 (m, 1H), 1.74-1.83 (m, 2H), 2.07 (m, 1H), |
| | 3.35-3.43 (m, 4H), 3.77 (s, 3H), 3.89 (s, 3H), 5.15 (s, 2H), 6.83 (d, 2H), 7.44 (d, 2H); LC-MS: m/z = 420 [M + H]⁺ |
| I-81 | δ = 3.02 (t, 2H), 3.39 (t, 2H), 3.81 (s, 3H), 4.06 (s, 3H), 6.91-6.97 (m, 3H), 7.27 (d, 2H), |
| | 7.42 (dd, 1H), 7.72 (d, 1H); LC-MS: m/z = 498 {[M(Cl³⁵) + H]⁺, 1 Cl} |
| I-82 | δ = 1.74-1.78 (m, 4H), 2.72 (t, 2H), 3.24 (t, 2H), 3.75 (s, 3H), 3.77 (s, 3H), 4.11 (s, 3H), |
| | 5.13 (s, 2H), 6.83 (d, 2H), 7.22-7.59 (m, 11H); LC-MS: m/z = 528 [M + H]⁺ |
| I-83 | δ = 3.00 (t, 2H), 3.51 (t, 2H), 3.76 (s, 3H), 3.78 (s, 3H), 4.13 (s, 3H), 5.11 (s, 2H), |
| | 6.80-6.85 (m, 4H), 6.95 (m, 1H), 7.05-7.10 (m, 2H), 7.20-7.29 (m, 3H), 7.35 (d, 1H); LC-MS: m/z = 584 {[M(Cl³⁵) + H]⁺, |
| | 2 Cl} |
| I-84 | δ = 1.65-1.80 (m, 4H), 2.82 (t, 2H), 3.11 (t, 2H), 3.80 (s, 3H), 4.13 (s, 3H), 7.08-7.14 (m, |
| | 2H), 7.30 (dd, 1H); LC-MS: m/z = 400 {[M(Cl³⁵) + H]⁺, 2 Cl} |
| I-85 | δ = 1.68-1.74 (m, 4H), 2.64 (t, 2H), 3.21 (t, 2H), 3.76 (s, 3H), 3.78 (s, 3H), 4.11 (s, 3H), |
| | 5.11 (s, 2H), 6.79-6.85 (m, 4H), 6.93 (d, 1H), 7.00 (d, 2H), 7.18 (t, 1H), 7.20-7.25 (m, |
| | 3H), 7.33 (t, 2H); LC-MS: m/z = 544 [M − H]⁺ |
| I-86 | δ = 1.73-1.74 (m, 4H), 2.71 (t, 2H), 3.13 (t, 2H), 3.80 (s, 3H), 4.09 (s, 3H), 7.26 (t, 2H), |
| | 7.32 (t, 1H) 7.43 (t, 2H), 7.51 (d, 2H), 7.57 (d, 2H); LC-MS: m/z = 408 [M − H]⁺ |
| I-87 | δ = 3.01 (t, 2H), 3.40 (t, 2H), 3.79 (s, 3H), 4.03 (s, 3H), 6.82-7.28 (m, 7H) |
| I-88 | δ = 3.00 (t, 2H), 3.40 (t, 2H), 3.82 (s, 3H), 4.07 (s, 3H), 6.91-6.95 (m, 4H), 7.18-7.25 (m, |
| | 4H); LC-MS: m/z = 430 {[M(Cl³⁵) + H]⁺, 1 Cl} |
| I-89 | log P (H₃PO₄) = 5.75; LC-MS: m/z = 550 {[M(Cl³⁵) + H]⁺, 1 Cl}^{c)} |
| I-90 | δ = 3.84 (s, 3H), 4.25 (s, 3H), 7.33 (t, 1H), 7.43 (t, 2H), 7.59 (m, 4H), 7.69 (d, 2H); LC-MS: |
| | m/z = 367 [M + H]⁺ |
| I-91 | δ = 1.69 (m, 2H), 1.76 (m, 2H), 2.73 (t, 2H), 3.11 (t, 2H), 3.82 (s, 3H), 4.07 (s, 3H), 7.16 (m, |
| | 2H), 7.35 (s, 1H); LC-MS: m/z = 400 {[M(Cl³⁵) + H]⁺, 2 Cl} |
| I-92 | δ = 3.78 (s, 3H), 3.80 (s, 3H), 4.14 (s, 3H), 5.08 (s, 2H), 6.03 (s, 2H), 6.82 (d, 2H), 7.09 (s, |
| | 1H), 7.21-7.26 (m, 4H); LC-MS: m/z = 440 [M + H]⁺ |
| I-93 | δ = 1.63-1.71 (m, 4H), 2.63 (t, 2H), 3.08 (t, 2H), 3.80 (s, 3H), 4.10 (s, 3H), 6.82-7.38 (m, |
| | 9H); LC-MS: m/z = 424 [M − H]⁺ |
| I-94 | δ = 2.63 (s, 3H), 3.37 (s, 3H), 3.88 (s, 3H), 6.77 (br. s, 1H); LC-MS: m/z = 214 [M + H]⁺ |
| I-95 | δ (DMSO-d₆) = 3.69 (s, 3H), 3.73 (s, 3H), 4.09 (s, 3H), 4.92, (s, 2H), 6.84 (d, 2H), 7.13 (d, |
| | 2H), 7.29-7.38 (m, 2H), 7.46 (t, 1H), 7.62 (d, 2H), 7.78 (d, 2H), 8.09 (d, 1H); LC-MS: m/z = 555 [M + H]⁺ |
| I-96 | δ = 3.00 (t, 2H), 3.39 (t, 2H), 3.80 (s, 3H), 4.05 (s, 3H), 6.75-7.38 (m, 7H); LC-MS: m/z = 464 {[M(Cl³⁵) + H]⁺, |
| | 2 Cl} |
| I-97 | δ (DMSO-d₆) = 3.73 (s, 3H), 3.75 (s, 3H), 4.12 (s, 3H), 5.16, (s, 2H), 6.89 (d, 2H), 7.24 (d, |
| | 2H), 7.74-7.79 (m, 4H); LC-MS: m/z = 512 [M + H]⁺ |
| I-98 | δ = 3.79 (s, 3H), 4.11 (s, 3H), 7.25-7.29 (m, 2H), 7.43 (m, 1H), 7.54 (d, 2H), 7.68 (d, 2H), |
| | 8.10 (d, 1H); LC-MS: m/z = 435 [M + H]⁺ |
| I-99 | δ = 0.96-0.88 (m, 6H), 1.16 (d, 3H), 1.19-1.26 (m, 2H), 1.36 (m, 1H), 1.76 (m, 1H), |
| | 3.76 (s, 3H), 3.78 (s, 3H), 4.11 (s, 3H), 4.15 (m, 1H), 5.10-5.17 (m, 2H), 6.83 (d, 2H), 7.25 (d, |
| | 2H); LC-MS: m/z = 418 [M + H]⁺ |
| I-100 | δ = 3.75 (s, 3H), 3.78 (s, 3H), 3.97 (s, 3H), 4.11 (s, 3H), 5.13 (s, 2H), 6.82 (d, 2H), 7.30 (d, |
| | 2H); LC-MS: m/z = 350 [M + H]⁺ |
| I-101 | δ = 0.91 (d, 3H), 1.00 (m, 2H), 1.40 (m, 1H), 1.57 (m, 2H), 1.77-1.86, (m, 4H), |
| | 3.44-3.51 (m, 1H) 3.77 (s, H), 3.88 (s, 3H), 5.15 (s, 2H), 6.83 (d, 2H), 7.44 (d, 2H); LC-MS: m/z = 402 [M + H]⁺ |
| I-102 | δ = 2.70 (s, 3H), 3.38 (s, H), 3.78 (s, 3H), 4.28 (s, 3H); LC-MS: m/z = 228 [M + H]⁺ |
| I-103 | δ = 1.18 (d, 6H), 3.76 (s, H), 3.79 (s, 3H), 4.12 (s, 3H), 4.21 (m, 1H), 5.13 (s, 2H), 6.85 (d, |
| | 2H), 7.24 (d, 2H); LC-MS: m/z = 362 [M + H]⁺ |
| I-104 | δ = 1.63 (d, 3H), 2.31-2.38 (m, 2H), 3.22 (t, 2H), 3.37 (s, 3H), 3.78 (s, 3H), 4.28 (s, 3H), |
| | 5.45-5.52 (m, 2H); LC-MS: m/z = 282 [M + H]⁺ |
| I-105 | δ = 0.95 (t, 3H), 1.41 (m, 2H), 1.66 (m, 2H), 3.19 (t, 2H), 3.75 (s, 3H), 3.79 (s, 3H), 4.11 (s, |
| | 3H), 5.13 (s, 2H), 6.84 (d, 2H), 7.25 (d, 2H); LC-MS: m/z = 376 [M + H]⁺ |
| I-106 | δ = 3.31 (s, 3H), 3.84 (s, 3H), 4.32 (s, 3H), 7.38-7.55 (m, 5H); LC-MS: m/z = 290 [M + H]⁺ |
| I-107 | δ = 3.74 (s, 3H), 3.80 (s, 3H), 4.14 (s, 3H), 4.48 (s, 2H), 5.13 (s, 2H), 6.85 (d, 2H), 7.18 (d, |
| | 2H), 7.21 (d, 2H), 7.44 (d, 2H); LC-MS: m/z = 488 {[M(Br⁷⁹) + H]⁺, 1 Br} |
| I-108 | δ = 1.18 (d, 6H), 3.77 (s, 3H), 4.08 (s, 3H), 4.19 (m, 1H), 5.21 (s, 2H), 7.23-7.35 (m, 5H); |
| | LC-MS: m/z = 332 [M + H]⁺ |
| I-109 | δ = 1.38-1.90 (m, 10H), 3.77 (s, 3H), 3.78 (s, 3H), 3.83 (s, 3H), 3.95 (m, 1H), 4.01 (s, 3H), |
| | 5.17 (s, 2H), 6.40 (dd, 1H), 6.45 (d, 1H), 6.71 (d, 1H); LC-MS: m/z = 430 [M − H]⁻ |
| I-110 | δ = 1.60-1.70 (m, 1H), 1.92-1.98 (m, 1H), 2.05-2.27 (m, 4H), 3.77 (s, 6H), 3.83 (s, 3H), |
| | 4.03 (s, 3H), 4.15-4.20 (m, 1H), 5.13-5.21 (m, 2H), 5.67-5.77 (m, 2H), 6.40 (dd, 1H), |
| | 6.45 (d, 1H), 6.72 (d, 1H); LC-MS: m/z = 428 [M − H]⁻ |
| I-111 | δ = 1.45-1.98, (m, 12H), 3.70-3.80 (m, 1H) 3.77 (s, H), 3.88 (s, 3H), 5.12 (s, 2H), |
| | 6.82 (d, 2H), 7.44 (d, 2H); LC-MS: m/z = 402 [M + H]⁺ |
| I-112 | δ = 1.21 (d, 6H), 3.77 (s, 3H), 3.84 (m, 1H), 3.89 (s, 3H), 5.12 (s, 2H), 6.82 (d, 2H), 6.88 (s, |
| | 1H), 7.45 (d, 2H); LC-MS: m/z = 348 [M + H]⁺ |
| I-113 | δ = 1.18 (d, 6H), 3.82 (s, 3H), 3.92 (m, 1H), 4.11 (s, 3H); LC-MS: m/z = 242 [M + H]⁺ |
| I-114 | δ = 0.84-1.93 (m, 10H), 3.62 (m, 1H), 3.82 (s, 3H), 4.09 (s, 3H); LC-MS: m/z = 282 [M + H]⁺ |
| I-115 | δ = 0.91 (d, 3H), 1.73-1.85 (m, 2H), 2.43 (dd, 1H), 2.70 (dd, 1H), 3.04-3.18 (m, 2H), |
| | 3.81 (s, 3H), 4.09 (s, 3H), 7.14-7.20 (m, 3H), 7.25-7.30 (m, 2H); LC-MS: m/z = 346 [M + H]⁺ |
| I-116 | δ = 1.53-1.96 (m, 12H), 3.75 (s, 3H), 3.79 (s, 3H), 4.08 (s, 3H), 4.17 (m, 1H), 5.14 (s, 2H), |
| | 6.86 (d, 2H), 7.22 (d, 2H); LC-MS: m/z = 416 [M + H]⁺ |
| I-117 | δ = 0.91 (d, 3H), 1.08-1.18 (m, 2H), 1.39-1.70 (m, 3H), 1.75-1.81 (m, 2H), |
| | 1.88-1.95 (m, 2H), 3.75 (s, 3H), 3.79 (s, 3H), 3.89 (m, 1H), 4.08 (s, 3H), 5.15 (s, 2H), 6.85 (d, 2H), |
| | 7.21 (d, 2H); LC-MS: m/z = 416 [M + H]⁺ |
| I-118 | δ = 1.12 (d, 3H), 1.62 (d, 3H), 2.04 (m, 1H), 2.44 (m, 1H), 3.77 (s, 3H), 3.78 (s, 3H), |
| | 3.83 (s, 3H), 4.02 (s, 3H), 4.19 (m, 1H), 5.16 (s, 2H), 5.40-5.46 (m, 2H), 6.39 (dd, 1H), 6.45 (d, |
| | 1H), 6.75 (d, 1H); LC-MS: m/z = 430 [M − H]⁻ |
| I-119 | δ (DMSO-d₆) = 1.55-1.78 (m, 6H), 1.85-1.93 (m, 2H), 3.69 (s, 3H), 3.73 (s, 3H), 4.12 (s, |
| | 3H), 4.28 (m, 1H), 5.05 (s, 2H), 6.87 (d, 2H), 7.19 (d, 2H); LC-MS: m/z = 388 [M + H]⁺ |
| I-120 | δ = 3.74 (s, 3H), 3.80 (s, 3H), 7.63 (d, 2H), 7.68 (d, 2H); LC-MS: m/z = 344 [M + H]⁺ |
| I-121 | δ = 0.91 (d, 3H), 0.95-1.07 (m, 2H), 1.41-1.68 (m, 3H), 1.76-1.95 (m, 4H), 3.62 (m, |
| | 1H), 3.81 (s, 3H), 4.14 (s, 3H); LC-MS: m/z = 296 [M + H]⁺ |
| I-122 | δ = 3.79 (s, 3H), 3.81 (s, 3H), 7.37 (d, 2H), 7.51 (d, 2H); LC-MS: m/z = 309 {[M(Cl³⁵) + H]⁺, |
| | 1 Cl} |
| I-123 | δ = 1.15 (d, 3H), 1.30 (t, 3H), 1.77 (s, 3H), 2.04 (m, 1H), 2.54 (m, 1H), 3.74 (s, 3H), 3.79 (s, |
| | 3H), 4.46 (q, 1H), 4.71 (m, 1H), 4.75 (m, 1H), 5.16 (s, 2H), 6.84 (d, 2H), 7.22 (d, 2H); LC- |
| | MS: m/z = 416 [M + H]⁺ |
| I-124 | δ = 1.12 (d, 3H), 1.29 (t, 3H), 1.40-1.88 (m, 4H), 1.95-2.00 (m, 2H), 2.55-2.70 (m, 1H), |
| | 3.74 (2s, 3H, isomer), 3.78 (s, 3H), 4.20-4.28 (m, 1H), 4.46 (q, 2H), 5.08-5.22 (m, 2H), |
| | 5.53-5.78 (m, 2H), 6.83 (d, 2H), 7.22 (d, 2H); LC-MS: m/z = 442 [M + H]⁺ |
| I-125 | δ = 1.50-1.65 (m, 8H), 1.75-1.84 (m, 2H), 1.91-1.98 (m, 2H), 3.81 (s, 3H), 3.88 (m, |
| | 1H), 4.13 (s, 3H); LC-MS: m/z = 296 [M + H]⁺ |
| I-126 | δ = 1.13 (d, 3H), 2.58 (dd, 1H), 3.17 (dd, 1H), 3.81 (s, 3H), 4.07 (s, 3H), 4.22 (m, 1H), |
| | 7.18-7.28 (m, 5H); LC-MS: m/z = 318 [M + H]⁺ |
| I-127 | δ = 0.94 (t, 3H), 1.41 (m, 2H), 1.68 (m, 2H), 3.07 (t, 2H), 3.82 (s, 3H), 4.10 (s, 3H); LC-MS: |
| | m/z = 256 [M + H]⁺ |
| I-128 | δ = 1.17 (d, 3H), 2.14 (m, 1H), 2.53 (m, 1H), 3.76 (s, 3H), 3.79 (s, 3H), 4.12 (s, 3H), |
| | 4.28 (m, 1H), 4.98-5.07 (m, 2H) 5.14 (s, 2H), 5.76-5.88 (m, 1H), 6.85 (d, 2H), 7.25 (d, 2H); |
| | LC-MS: m/z = 388 [M + H]⁺ |
| I-129 | δ = 0.91 (t, 3H), 1.15 (d, 3H), 1.30-1.40 (m, 3H), 1.77 (m, 1H), 3.82 (s, 3H), 3.89 (m, 1H), |
| | 4.14 (s, 3H); LC-MS: m/z = 270 [M + H]⁺ |
| I-130 | δ = 1.05-1.13 (m, 2H), 1.28-1.35 (m, 2H), 3.47 (m, 1H), 3.82 (s, 3H), 4.16 (s, 3H); LC- |
| | MS: m/z = 240 [M + H]⁺ |
| I-131 | δ = 1.51-2.35 (m, 6H), 3.75-3.85 (m, 1H), 3.82 (s, 3H), 4.11 (s, 3H), 5.70-5.78 (m, 2H); |
| | LC-MS: m/z = 280 [M + H]⁺ |
| I-132 | δ = 1.20 (d, 3H), 2.19 (m, 1H), 2.52 (m, 1H), 3.78 (s, 3H), 3.86 (m, 1H), 3.89 (s, 3H), |
| | 5.00-5.16 (m, 4H), 5.71-5.81 (m, 1H), 6.82 (d, 2H), 7.45 (d, 2H); LC-MS: m/z = 374 [M + H]⁺ |
| I-133 | δ = 1.12 (d, 3H), 2.20 (m, 1H), 2.53 (m, 1H), 3.76 (s, 3H), 3.83 (s, 3H), 3.91 (s, 3H), |
| | 5.00-5.09 (m, 2H), 5.12-5.21 (m, 2H), 5.73-5.83 (m, 1H), 6.39 (dd, 1H), 6.45 (d, 1H), 6.85 (d, |
| | 1H); LC-MS: m/z = 402 [M − H]⁻ |
| I-134 | δ = 0.91-0.98 (2t, 6H), 1.21 (m, 1H), 1.48 (m, 1H), 1.68 (m, 1H), 3.21 (m, 1H), 3.33 (m, |
| | 1H), 3.77 (s, 3H), 3.78 (s, 3H), 4.03 (s, 3H), 5.15 (s, 2H), 6.85 (d, 2H), 7.21 (d, 2H), |
| | 10.18 (br. t, 1H); LC-MS: m/z = 405 [M + H]⁺ |
| I-135 | δ (DMSO-d₆) = 0.81 (t, 3H), 1.18-1.28 (m, 2H), 1.20 (d, 3H), 1.47-1.57 (m, 2H), |
| | 3.04 (m, 1H), 3.72 (s, 6H), 4.13 (s, 3H), 5.13 (s, 2H), 6.88 (d, 2H), 7.20-7.25 (m, 4H), 7.33 (m, |
| | 1H), 7.79 (m, 1H); LC-MS: m/z = 481 [M + H]⁺ |
| I-136 | δ = 1.15 (d, 3H), 2.14 (m, 1H), 2.53 (m, 1H), 3.78 (s, 3H), 3.83 (s, 3H), 4.03 (s, 3H), |
| | 4.25 (m, 1H), 4.95-5.05 (m, 2H), 5.17 (s, 2H), 5.81 (m, 1H), 6.39 (dd, 1H), 6.45 (d, 1H), |
| | 6.75 (d, 1H); LC-MS: m/z = 416 [M − H]⁻ |
| I-137 | δ = 1.15 (t, 3H), 3.19 (q, 2H), 3.77 (s, 3H), 3.84 (s, 3H), 4.04 (s, 3H), 5.16 (s, 2H), 6.39 (dd, |
| | 1H), 6.45 (d, 1H), 6.75 (d, 1H); LC-MS: m/z = 376 [M − H]⁻ |
| I-138 | δ = 0.89 (d, 3H), 0.90 (d, 3H), 1.14 (d, 3H), 1.22 (m, 1H), 1.62 (m, 1H), 1.72 (m, 1H), |
| | 3.83 (s, 3H), 3.99 (m, 1H), 4.09 (s, 3H); LC-MS: m/z = 284 [M + H]⁺ |
| I-139 | δ = 0.85 (t, 3H), 1.20-1.60 (m, 7H), 3.06 (m, 1H), 3.82 (s, 3H), 4.21 (s, 3H), |
| | 7.18-7.29 (m, 4H), 7.81 (br. d, 1H); LC-MS: m/z = 361 [M + H]⁺ |
| I-140 | δ^{b)} = 1.15 (d, 3H), 2.61 (dd, 1H), 3.19 (dd, 1H), 3.75 (s, 3H), 3.79 (s, 3H), 4.12 (s, 3H), |
| | 4.55 (m, 1H), 5.10-5.15 (m, 2H), 6.85 (d, 2H), 7.24 (d, 2H), 7.40 (d, 2H), 7.50 (d, 2H); LC-MS: |
| | m/z = 506 [M + H]⁺ |
| I-141 | δ = 1.19 (d, 3H), 2.65 (m, 1H), 3.22 (m, 1H), 3.79 (s, 3H), 4.10 (s, 3H), 4.27 (m, 1H), |
| | 7.31 (d, 2H), 7.49 (d, 2H); LC-MS: m/z = 386 [M + H]⁺ |
| I-142 | δ = 1.14-1.19 (m, 6H), 2.14 (m, 1H), 2.53 (m, 1H), 3.74 (s, 3H), 3.76 (s, 3H), 3.82 (s, 3H), |
| | 4.38 (q, 2H), 4.96-5.05 (m, 2H), 5.19 (s, 2H), 5.81 (m, 1H), 6.39 (dd, 1H), 6.45 (d, 1H), |
| | 6.71 (d, 1H); LC-MS: m/z = 430 [M − H]⁻ |
| I-143 | δ = 1.19 (d, 3H), 2.47 (dd, 1H), 2.80 (dd, 1H), 3.73 (s, 3H), 3.75 (s, 3H), 3.79 (s, 3H), |
| | 4.11 (s, 3H), 4.49 (m, 1H), 5.13 (s, 2H), 5.56 (m, 1H), 6.17 (m, 1H), 6.85 (d, 2H), 7.24 (d, 2H); |
| | LC-MS: m/z = 446 [M + H]⁺ |
| I-144 | δ = 0.91-0.97 (m, 6H), 1.20-1.25 (m, 1H), 1.42-1.50 (m, 1H), 1.63-1.68 (m, 1H), |
| | 3.24 (m, 1H), 3.35 (m, 1H), 3.80 (s, 3H), 4.17 (s, 3H); LC-MS: m/z = 285 [M + H]⁺ |
| I-145 | δ = 0.90 (t, 3H), 1.15 (d, 3H), 1.21-1.28 (m, 3H), 1.45 (t, 3H), 1.78 (m, 1H), 3.79 (s, 3H), |
| | 3.99 (m, 1H), 4.55 (q, 2H); LC-MS: m/z = 284 [M + H]⁺ |
| I-146 | δ = 3.79 (s, 3H), 3.82 (s, 3H), 3.87 (s, 3H), 3.90 (s, 3H), 3.96 (s, 3H); LC-MS: m/z = 366 [M + H]⁺ |
| I-147 | δ = 1.15 (d, 3H), 1.19 (d, 3H), 1.49 (m, 0.6H, diastereomer A), 1.71 (m, 0.4H, diastereomer |
| | B), 1.98 (m, 0.4H, diastereomer B), 2.25 (m, 0.6H, diastereomer A), 2.51 (m, 1H), 3.62 (s, |
| | 1.2H, diastereomer B), 3.66 (s, 1.8H, diastereomer A), 3.81 (s, 3H), 3.91-4.12 (m, 1H), |
| | 4.14 (s, 3H); LC-MS: m/z = 328 [M + H]⁺ |
| I-148 | δ = 1.18 (d, 3H), 2.80 (dd, 1H), 3.09 (dd, 1H), 3.81 (s, 3H), 4.08 (s, 3H), 4.22 (m, 1H), |
| | 6.95-7.06 (m, 2H), 7.13-7.25 (m, 2H); LC-MS: m/z = 336 [M + H]⁺ |
| I-149 | δ = 0.94-1.20 (m, 4H), 1.05 (d, 3H), 1.61-1.73 (m, 6H), 3.71 (s, 3H), 3.87 (m, 1H), |
| | 4.00 (s, 3H); LC-MS: m/z = 310 [M + H]⁺ |
| I-150 | δ = 1.14 (d, 3H), 1.61 (s, 3H), 1.68 (s, 3H), 2.14 (m, 1H), 2.40 (m, 1H), 3.76 (s, 3H), 3.78 (s, |
| | 3H), 4.11 (s, 3H), 4.18 (m, 1H), 5.13 (s, 2H), 6.84 (d, 2H), 7.25 (m, 2H); LC-MS: m/z = 416 [M + H]⁺ |
| I-151 | δ = 1.15 (d, 3H), 1.58 (s, 3H), 1.61 (s, 3H), 1.66 (s, 3H), 1.93-2.07 (m, 4H), 2.15 (m, 1H), |
| | 2.40 (m, 1H), 3.75 (s, 3H), 3.78 (s, 3H), 4.11 (s, 3H), 4.19 (m, 1H), 5.08 (m, 1H), 5.13 (s, |
| | 2H), 5.18 (m, 1H), 6.84 (d, 2H), 7.25 (m, 2H); LC-MS: m/z = 484 [M + H]⁺ |
| I-152 | δ = 1.00 (t, 3H), 1.72 (m, 2H), 2.94 (t, 2H), 3.77 (s, 3H), 3.89 (s, 3H), 5.12 (s, 2H), 6.83 (d, |
| | 2H), 7.45 (d, 2H); LC-MS: m/z = 348 [M + H]⁺ |
| I-153 | δ = 0.86 (d, 3H), 0.88 (d, 3H), 1.16 (d, 3H), 1.20 (m, 1H), 1.35 (m, 1H), 1.51 (m, 1H), |
| | 1.78 (m, 1H), 3.83 (m, 1H), 3.82 (s, 3H), 4.11 (s, 3H); LC-MS: m/z = 298 [M + H]⁺ |
| I-154 | δ = 1.14 (d, 3H), 1.30 (s, 9H), 2.55 (dd, 1H), 3.14 (dd, 1H), 3.82 (s, 3H), 4.08 (s, 3H), |
| | 4.18 (m, 1H), 7.16 (d, 2H), 7.28 (d, 2H); LC-MS: m/z = 374 [M + H]⁺ |
| I-155 | δ = 1.25 (d, 3H), 1.89 (m, 1H), 2.35 (m, 1H), 3.79 (s, 3H), 3.96-4.05 (m, 2H), 4.09 (s, 3H), |
| | 4.14 (m, 1H), 6.83 (d, 2H), 6.90 (t, 1H), 7.22-7.26 (m, 2H); LC-MS: m/z = 348 [M + H]⁺ |
| I-156 | δ^{b)} = 1.19 (d, 3H), 2.88 (dd, 1H), 3.21 (dd, 1H), 3.79 (s, 3H), 4.06 (s, 3H), 4.31 (m, 1H), |
| | 7.10-7.15 (m, 2H), 7.22 (m, 1H), 7.31 (m, 1H); LC-MS: m/z = 352 {[M(Cl³⁵) + H]⁺, 1 Cl} |
| I-157 | δ = 1.16 (d, 3H), 2.20 (m, 1H), 2.50 (m, 1H), 3.75 (s, 3H), 3.77 (s, 3H), 4.11 (2s, 5H), |
| | 4.49 (s, 2H), 5.12 (s, 2H), 5.59-5.68 (m, 2H), 6.84 (d, 2H), 7.24-7.36 (m, 5H); LC-MS: m/z = 508 [M + H]⁺ |
| I-158 | δ = 1.16 (d, 3H), 1.38-1.48 (m, 2H), 1.63 (m, 1H), 1.85 (m, 1H), 2.58-2.65 (m, 2H), |
| | 3.81 (s, 3H), 3.89 (m, 1H), 4.10 (s, 3H); LC-MS: m/z = 346 [M + H]⁺ |
| I-159 | log P (H₃PO₄) = 5.73; LC-MS: m/z = 368 [M + H]⁺ |
| I-160 | δ = 0.80, 0.88 (2d, 3H, 2 diastereomers), 1.11, 1.17 (2d, 3H, 2 diastereomers), |
| | 1.63-2.01 (m, 2H, 2 diastereomers), 2.29, 2.38 (2dd, 1H, 2 diastereomers), 2.64, 2.75 (2dd, 1H, 2 |
| | diastereomers), 3.80 (s, 3H), 4.10-4.19 (m, 1H), 4.13 (s, 3H), 7.08-7.19 (m, 3H), |
| | 7.21-7.27 (m, 2H); LC-MS: m/z = 360 [M + H]⁺ |
| I-161 | δ (DMSO-d₆) = 3.63 (s, 6H), 7.28-7.34 (m, 2H), 7.46-7.58 (m, 2H); LC-MS: m/z = 294 [M + H]⁺ |
| I-162 | δ = 1.15 (t, 3H), 3.17 (q, 2H), 3.78 (s, 3H), 4.23 (s, 3H), 4.61 (m, 2H), 5.08-5.19 (m, 2H), |
| | 5.89 (m, 1H); LC-MS: m/z = 268 [M + H]⁺ |
| I-163 | δ = 0.80-0.90 (m, 6H), 1.10-1.92 (m, 13H), 1.14 (t, 3H), 3.83 (s, 3H), 3.90-4.13 (m, |
| | 1H), 4.12 (s, 3H); LC-MS: m/z = 354 [M + H]⁺ |
| I-164 | log P (H₃PO₄) = 5.41; LC-MS: m/z = 366 [M + H]⁺ |
| I-165 | δ = 3.80 (s, 3H), 4.02 (s, 3H), 4.09 (s, 3H); LC-MS: m/z = 230 [M + H]⁺ |
| I-166 | δ = 1.01 (t, 3H), 1.70 (m, 2H), 3.17 (t, 2H), 3.76 (s, 3H), 3.78 (s, 3H), 4.11 (s, 3H), 5.13 (s, |
| | 2H), 6.84 (d, 2H), 7.24 (d, 2H); LC-MS: m/z = 360 [M − H]⁻ |
| I-167 | LC-MS: m/z = 410 [M + H]^{+c)} |
| I-168 | δ = 3.82 (s, 3H), 3.93 (s, 3H), 6.98 (d, 2H), 7.08 (d, 2H), 7.19 (t, 1H), 7.40 (t, 2H), 7.63 (d, |
| | 2H); LC-MS: m/z = 368 [M + H]⁺ |
| I-169 | δ (DMSO-d₆) = 2.49-2.70 (m, 4H), 3.70 (s, 3H), 3.73 (s, 3H), 4.12 (s, 3H), 4.62 (m, 1H), |
| | 5.06 (s, 2H), 5.65 (m, 2H), 6.87 (d, 2H), 7.20 (d, 2H); LC-MS: m/z = 386 [M + H]⁺ |
| I-170 | δ = 0.80-0.86 (m, 6H), 1.10 (d, 3H), 1.16-1.38 (m, 6H), 1.74 (m, 1H), 3.71 (s, 3H), |
| | 3.75 (s, 3H), 4.11 (s, 3H), 4.38 (m, 1H), 5.04-5.15 (m, 2H), 6.85 (d, 2H), 7.21 (d, 2H); LC-MS: |
| | m/z = 432 [M + H]⁺ |
| I-171 | δ = 1.15 (d, 3H), 2.55 (dd, 1H), 3.13 (dd, 1H), 3.81 (s, 3H), 4.13 (s, 3H), 4.21 (m, 1H), |
| | 6.88-6.93 (m, 4H), 7.19 (d, 2H), 7.26 (d, 2H); LC-MS: m/z = 444 [M + H]⁺ |
| I-172 | δ = 1.21 (d, 3H), 3.18 (dd, 1H), 3.35 (dd, 1H), 3.77 (s, 3H), 3.79 (s, 3H), 4.10 (s, 3H), |
| | 4.65 (m, 1H), 5.05-5.11 (m, 2H), 6.81 (d, 2H), 7.05 (t, 1H), 7.20-7.25 (m, 4H); LC-MS: m/z = 506 {[M(Cl³⁵) + H]⁺, |
| | 2 Cl} |
| I-173 | δ = 1.13 (d, 3H), 2.56 (dd, 1H), 3.16 (dd, 1H), 3.75 (s, 3H), 3.78 (s, 3H), 4.10 (s, 3H), |
| | 4.52 (m, 1H), 5.14 (s, 2H), 6.86 (d, 2H), 7.14-7.29 (m, 7H); LC-MS: m/z = 438 [M + H]⁺ |
| I-174 | δ = 1.20 (d, 3H), 3.20 (dd, 1H), 3.34 (dd, 1H), 3.79 (s, 3H), 4.04 (s, 3H), 4.38 (m, 1H), |
| | 7.08 (t, 1H), 7.27 (d, 2H); LC-MS: m/z = 386 {[M(Cl³⁵) + H]⁺, 2 Cl} |
| I-175 | δ = 1.53 (d, 3H), 2.30 (s, 3H), 3.76 (s, 3H), 3.86 (s, 3H), 5.10 (s, 2H), 6.80 (d, 2H), 7.11 (d, |
| | 2H), 7.26 (d, 2H), 7.41 (d, 2H); LC-MS: m/z = 424 [M + H]⁺ |
| I-176 | δ = 1.53 (d, 3H), 3.76 (s, 3H), 3.85 (s, 3H), 5.10 (s, 2H), 6.80 (d, 2H), 7.25 (d, 2H), 7.30 (d, |
| | 2H), 7.41 (d, 2H); LC-MS: m/z = 444 {[M(Cl³⁵) + H]⁺, 1 Cl} |
| I-177 | δ = 0.88 (t, 3H), 1.18 (d, 3H), 1.25-1.35 (m, 4H), 1.43 (m, 1H), 1.78 (m, 1H), 3.79 (m, |
| | 1H), 3.78 (s, 3H), 3.89 (s, 3H), 5.12 (s, 2H), 6.83 (d, 2H), 7.45 (d, 2H); LC-MS: m/z = 390 [M + H]⁺ |
| I-178 | δ = 0.87 (t, 3H), 1.19 (d, 3H), 1.23-1.35 (m, 6H), 1.42 (m, 1H), 1.76 (m, 1H), |
| | 3.77-3.82 (m, 1H), 3.78 (s, 3H), 3.89 (s, 3H), 5.13 (s, 2H), 6.83 (d, 2H), 7.45 (d, 2H); LC-MS: m/z = 404 [M + H]⁺ |
| I-179 | δ = 0.85-0.95 (m, 6H), 1.20-1.80 (m, 8H), 3.70-3.80 (m, 1H), 3.80 (s, 3H), 3.89 (s, 3H), |
| | 5.13 (s, 2H), 6.83 (d, 2H), 7.45 (d, 2H); LC-MS: m/z = 404 [M + H]⁺ |
| I-180 | δ = 1.31 (d, 3H), 2.20 (m, 1H), 2.79 (m, 1H), 3.78 (s, 3H), 3.89 (s, 3H), 4.20 (m, 1H), |
| | 5.10 (d, 1H), 5.16 (d, 1H), 6.83 (d, 2H), 7.44 (d, 2H); LC-MS: m/z = 416 [M + H]⁺ |
| I-181 | δ = 1.46 (d, 3H), 3.72 (s, 3H), 3.79 (s, 3H), 4.07 (s, 3H), 5.03 (d, 1H), 5.12 (d, 1H), 5.59 (q, |
| | 1H), 6.81 (d, 2H), 7.10 (d, 2H), 7.25 (d, 2H), 7.34 (d, 2H); LC-MS: m/z = 458 {[M(Cl³⁵) + H]⁺, |
| | 1 Cl} |
| I-182 | δ = 1.27 (t, 3H), 1.46 (d, 3H), 3.71 (s, 3H), 3.79 (s, 3H), 4.40-4.49 (m, 2H), 5.06 (d, 1H), |
| | 5.14 (d, 1H), 5.59 (q, 1H), 6.80 (d, 2H), 7.09 (d, 2H), 7.25 (d, 2H), 7.34 (d, 2H); LC-MS: |
| | m/z = 472 {[M(Cl³⁵) + H]⁺, 1 Cl} |
| I-183 | δ = 1.48 (d, 3H), 2.32 (s, 3H), 3.71 (s, 3H), 3.78 (s, 3H), 4.06 (s, 3H), 5.05-5.13 (m, 2H), |
| | 5.62 (q, 1H), 6.80 (d, 2H), 7.11 (d, 2H), 7.15 (d, 2H), 7.32 (d, 2H); LC-MS: m/z = 438 [M + H]⁺ |
| I-184 | δ = 0.85-0.91 (m, 3H, diastereomers), 1.05-2.30 (m, 13H), 3.75 (s, 3H), 3.78 (s, 3H), |
| | 3.85-3.95 (m, 1H, diastereomers), 4.08 (s, 3H), 5.15 (s, 2H), 6.84 (d, 2H), 7.22 (d, 2H); LC- |
| | MS: m/z = 444 [M + H]⁺ |
| I-185 | δ = 1.29 (d, 3H), 2.17 (m, 1H), 2.82 (m, 1H), 3.76 (s, 3H), 3.79 (s, 3H), 4.13 (s, 3H), |
| | 4.58 (q, 1H), 5.11 (d, 1H), 5.17 (d, 1H), 6.83 (d, 2H), 7.25 (d, 2H); LC-MS: m/z = 430 [M + H]⁺ |
| I-186 | δ = 0.87 (t, 3H), 1.16 (d, 3H), 1.24-1.35 (m, 6H), 1.65-1.80 (m, 2H), 3.76 (s, 3H), |
| | 3.78 (s, 3H), 4.10 (s, 3H), 4.18 (q, 1H), 5.14 (s, 2H), 6.83 (d, 2H), 7.25 (d, 2H); LC-MS: m/z = 418 [M + H]⁺ |
| I-187 | δ = 0.85-0.95 (m, 6H), 1.20-1.60 (m, 5H), 1.70-1.80 (m, 3H), 3.76 (s, 3H), 3.78 (s, 3H), |
| | 4.10 (s, 3H), 4.23 (q, 1H), 5.10-5.18 (m, 2H), 6.83 (d, 2H), 7.24 (d, 2H); LC-MS: m/z = 418 [M + H]⁺ |
| I-188 | δ = 1.49 (d, 3H), 3.73 (s, 3H), 3.79 (s, 3H), 4.11 (s, 3H), 5.03 (d, 1H), 5.12 (d, 1H), 5.59 (q, |
| | 1H), 6.83 (d, 2H), 7.14 (d, 2H), 7.25 (d, 1H), 7.69 (dd, 1H), 8.43 (d, 1H); LC-MS: m/z = 459 {[M(Cl³⁵) + H]⁺, |
| | 1 Cl} |
| I-189 | log P (H₃PO₄) = 3.74, 4.04 (2 diastereomers); LC-MS: m/z = 432 [M + H]⁺ |
| I-190 | δ = 1.49 (d, 3H), 3.73 (s, 3H), 3.78 (s, 3H), 4.09 (s, 3H), 5.06-5.13 (m, 2H), 5.65 (q, 1H), |
| | 6.82 (d, 2H), 6.90-6.95 (m, 6H), 7.16 (d, 2H), 7.39 (d, 2H); LC-MS: m/z = 450 {[M(Cl³⁵) + H]⁺, |
| | 1 Cl} |
| I-191 | δ = 1.34 (s, 9H), 3.78 (s, 3H), 3.80 (s, 3H), 4.12 (s, 3H), 5.10 (s, 2H), 6.83 (d, 2H), 7.22 (d, |
| | 2H), 7.42 (d, 2H), 7.59 (d, 2H); LC-MS: m/z = 452 [M + H]⁺ |
| I-192 | δ = 0.90-0.95 (m, 3H, diastereomers), 1.01-1.12 (m, 3H), 1.25-2.00 (m, 6H), 3.75 (s, |
| | 3H), 3.78 (s, 3H), 3.95-4.03 (m, 1H, diastereomers), 4.08 (s, 3H), 5.15 (s, 2H), 6.84 (d, |
| | 2H), 7.22 (d, 2H); LC-MS: m/z = 416 [M + H]⁺ |
| I-193 | δ = 0.88 (t, 3H), 1.16 (d, 3H), 1.27-1.35 (m, 4H), 1.57 (m, 1H), 1.77 (m, 1H), 3.76 (s, 3H), |
| | 3.78 (s, 3H), 4.10 (s, 3H), 4.18 (q, 1H), 5.14 (s, 2H), 6.83 (d, 2H), 7.24 (d, 2H); LC-MS: m/z = 404 [M + H]⁺ |
| I-194 | log P (HCO₂H) = 5.32; LC-MS: m/z = 444 [M + H]^{+c)} |
| I-195 | δ = 1.35 (s, 9H), 3.82 (s, 3H), 4.01 (s, 3H), 7.43 (d, 2H), 7.57 (d, 2H); LC-MS: m/z = 332 [M + H]⁺ |
| I-196 | δ = 1.13-2.28 (m, 8H), 3.20-3.28 (m, 0.6H), 3.33, 3.36 (2s, 3H, diastereomers), |
| | 3.61-3.72 (m, 1H, diastereomers), 3.80, 3.81 (2s, 3H, diastereomers), 4.00-4.07 (m, 0.4H), 4.12, |
| | 4.13 (2s, 3H, diastereomers); LC-MS: m/z = 312 [M + H]⁺ |
| I-197 | δ = 1.28 (d, 3H), 2.16 (m, 1H), 2.84 (m, 1H), 3.81 (s, 3H), 4.16 (s, 3H), 4.35 (q, 1H); LC- |
| | MS: m/z = 310 [M + H]⁺ |
| I-198 | δ (DMSO-d₆) = 0.85 (t, 3H), 1.08 (d, 3H), 1.20-1.30 (m, 7H), 1.69 (m, 1H), 3.62 (s, 3H), |
| | 3.88 (m, 1H), 3.92 (s, 3H); LC-MS: m/z = 298 [M + H]⁺ |
| I-199 | δ = 1.23 (d, 3H), 2.96 (dd, 1H), 3.24 (dd, 1H), 3.81 (s, 3H), 4.09 (s, 3H), 4.20 (q, 1H), |
| | 6.84 (d, 1H), 7.10 (d, 1H); LC-MS: m/z = 358 {[M(Cl³⁵) + H]⁺, 1 Cl} |
| I-200 | δ = 0.89 (t, 3H), 0.93-1.02 (m, 2H), 1.15-1.22 (m, 2H), 1.23-1.53 (m, 4H), |
| | 1.82-1.93 (m, 5H), 3.61 (m, 1H), 3.81 (s, 3H), 4.12 (s, 3H); LC-MS: m/z = 324 [M + H]⁺ |
| I-201 | δ = 1.48 (d, 3H), 2.30 (s, 3H), 3.77 (s, 3H), 4.08 (s, 3H), 5.24 (q, 1H), 7.10 (d, 2H), 7.23 (d, |
| | 2H); LC-MS: m/z = 318 [M + H]⁺ |
| I-202 | log P (H₃PO₄) = 6.00; LC-MS: m/z = 360 [M + H]^{+c)} |
| I-203 | δ = 0.93 (d, 3H), 1.30-1.90 (m, 9H), 3.72 (m, 1H), 3.81 (s, 3H), 4.14 (s, 3H); LC-MS: m/z = 296 [M + H]⁺ |
| I-204 | δ = 1.47 (d, 3H), 3.78 (s, 3H), 4.15 (s, 3H), 5.30 (q, 1H), 7.25 (d, 2H), 7.30 (d, 2H); LC-MS: |
| | m/z = 338 {[M(Cl³⁵) + H]⁺, 1 Cl} |
| I-205 | δ = 1.42 (t, 3H), 1.48 (d, 3H), 3.77 (s, 3H), 4.55 (q, 2H), 5.40 (q, 1H), 7.25 (d, 2H), 7.32 (d, |
| | 2H); LC-MS: m/z = 352 {[M(Cl³⁵) + H]⁺, 1 Cl} |
| I-206 | δ = 1.47 (d, 3H), 3.79 (s, 3H), 4.11 (s, 3H), 5.23 (q, 1H), 7.19 (dd, 1H), 7.35 (d, 1H), |
| | 7.45 (d, 1H); LC-MS: m/z = 372 {[M(Cl³⁵) + H]⁺, 2 Cl} |
| I-207 | δ = 1.47 (d, 3H), 3.80 (s, 3H), 4.07 (s, 3H), 5.41 (q, 1H), 7.11 (d, 1H), 7.19 (dd, 1H), |
| | 7.40 (d, 1H); LC-MS: m/z = 372 {[M(Cl³⁵) + H]⁺, 2 Cl} |
| I-208 | log P (H₃PO₄) = 3.55; LC-MS: m/z = 298 [M + H]⁺ |
| I-209 | δ = 1.49 (d, 3H), 3.79 (s, 3H), 4.08 (s, 3H), 5.25 (q, 1H), 6.88-6.95 (m, 4H), |
| | 7.26-7.34 (m, 4H); LC-MS: m/z = 430 {[M(Cl³⁵) + H]⁺, 1 Cl} |
| I-210 | δ = 1.14 (d, 3H), 2.55 (dd, 1H), 3.13 (dd, 1H), 3.81 (s, 3H), 4.09 (s, 3H), 4.17 (m, 1H), |
| | 7.18 (d, 2H), 7.23 (d, 2H); LC-MS: m/z = 352 {[M(Cl³⁵) + H]⁺, 1 Cl} |
| I-211 | δ = 0.88 (t, 6H), 1.31 (m, 4H), 1.43 (m, 2H), 1.72 (m, 2H), 3.82 (s, 3H), 3.98 (m, 1H), |
| | 4.10 (s, 3H); LC-MS: m/z = 298 [M + H]⁺ |
| I-212 | δ = 0.83-0.90 (m, 6H), 1.10-1.93 (m, 16H), 3.82 (s, 3H), 4.11 (s, 3H), 4.31 (m, 1H); LC- |
| | MS: m/z = 354 [M + H]⁺ |
| I-213 | δ = 0.92-1.75 (m, 11H), 1.09 (d, 3H), 1.44 (t, 3H), 3.80 (s, 3H), 3.91 (m, 1H), 4.53 (q, 2H); |
| | LC-MS: m/z = 324 [M + H]⁺ |
| I-214 | δ = 1.17 (d, 3H), 2.33 (s, 3H), 2.68 (dd, 1H), 3.12 (dd, 1H), 3.80 (s, 3H), 4.06 (s, 3H), |
| | 4.28 (m, 1H), 7.06-7.19 (m, 4H); LC-MS: m/z = 332 [M + H]⁺ |
| I-215 | δ = 1.49 (d, 3H), 3.78 (s, 3H), 4.16 (s, 3H), 5.31 (q, 1H), 7.27 (d, 1H), 7.66 (dd, 2H), |
| | 8.39 (d, 1H); LC-MS: m/z = 338 {[M(Cl³⁵) + H]⁺, 1 Cl} |
| I-216 | δ = 1.86 (m, 1H), 2.18 (m, 1H), 2.92 (dd, 2H), 2.97-3.02 (m, 2H), 3.79 (s, 3H), 3.92 (s, |
| | 3H), 4.07 (m, 1H), 7.09-7.15 (m, 4H); LC-MS: m/z = 330 [M + H]⁺ |
| I-217 | δ = 1.15 (d, 3H), 2.64 (dd, 1H), 3.22 (dd, 1H), 3.81 (s, 3H), 4.08 (s, 3H), 4.24 (m, 1H), |
| | 7.35-7.46 (m, 3H), 7.49 (s, 1H); LC-MS: m/z = 386 [M + H]⁺ |
| I-218 | δ = 1.17 (d, 3H), 2.24 (s, 3H), 2.32 (s, 3H), 2.35 (dd, 1H), 2.81 (dd, 1H), 3.80 (s, 3H), |
| | 4.12 (s, 3H), 4.21 (m, 1H); LC-MS: m/z = 337 [M + H]⁺ |
| I-219 | δ = 0.85-0.89 (m, 6H), 1.08-1.43 (m, 3H), 1.59 (m, 1H), 1.90 (m, 1H), 3.81 (s, 3H), |
| | 4.00-4.14 (m, 1H), 4.14 (2s, 3H, diastereomers); LC-MS: m/z = 298 [M + H]⁺ |
| I-220 | δ = 1.50 (d, 3H), 3.78 (s, 3H), 4.10 (s, 3H), 5.30 (m, 1H), 7.20-7.36 (m, 5H); LC-MS: m/z = 304 [M + H]⁺ |
| I-221 | δ = 3.36 (s, 3H), 3.75 (s, 3H), 7.51-7.63 (m, 2H), 7.82-7.94 (m, 4H), 8.05 (s, 1H); LC- |
| | MS: m/z = 326 [M + H]⁺ |
| I-222 | δ = 0.86-0.88 (2d, 6H), 1.15 (d, 3H), 1.15-1.22 (m, 2H), 1.38 (m, 1H), 1.44 (t, 3H), |
| | 1.51 (m, 1H), 1.78 (m, 1H), 3.80 (s, 3H), 3.92 (m, 1H), 4.53 (q, 2H); LC-MS: m/z = 312 [M + H]⁺ |
| I-223 | δ = 1.20 (d, 3H), 2.90 (dd, 1H), 3.18 (dd, 1H), 3.75 (s, 3H), 3.78 (s, 3H), 4.10 (s, 3H), |
| | 4.61 (m, 1H), 5.08-5.16 (m, 2H), 6.83 (d, 2H), 7.09-7.17 (m, 2H), 7.23 (d, 2H), |
| | 7.30-7.38 (m, 2H); LC-MS: m/z = 472 {[M(Cl³⁵) + H]⁺, 1 Cl} |
| I-224 | δ = 1.20 (d, 3H), 1.50-1.62 (m, 3H), 1.82 (m, 1H), 3.31 (s, 3H), 3.37 (t, 2H), 3.77 (s, 3H), |
| | 3.88 (s, 3H), 5.12 (s, 2H), 6.83 (d, 2H), 7.44 (d, 2H); LC-MS: m/z = 406 [M + H]⁺ |
| I-225 | δ = 2.68-2.73 (m, 4H), 3.77 (s, 3H), 3.88 (s, 3H), 4.40 (m, 1H), 5.13 (s, 2H), |
| | 5.67-5.69 (m, 2H), 6.83 (d, 2H), 7.44 (d, 2H); LC-MS: m/z = 372 [M + H]⁺ |
| I-226 | δ = 1.01-1.12 (m, 2H), 1.16 (d, 3H), 1.31-1.38 (m, 2H), 1.46-1.61 (m, 5H), |
| | 1.68-1.78 (m, 4H), 3.75 (s, 3H), 3.78 (s, 3H), 4.11 (s, 3H), 4.16 (m, 1H), 5.11-5.18 (m, 2H), 6.84 (d, |
| | 2H), 7.25 (d, 2H); LC-MS: m/z = 444 [M + H]⁺ |
| I-227 | δ = 0.86-0.92 (m, 9H), 1.22-1.29 (m, 2H), 1.50-1.78 (m, 3H), 3.83 (s, 3H), 3.99 (m, |
| | 1H), 4.09 (s, 3H); LC-MS: m/z = 298 [M + H]⁺ |
| I-228 | δ (DMSO-d₆) = 0.84 (s, 9H), 1.06 (d, 3H), 1.10-1.30 (m, 3H), 1.68 (m, 1H), 3.60 (s, 3H), |
| | 3.89 (s, 3H), 3.91 (m, 1H); LC-MS: m/z = 312 [M + H]⁺ |
| I-229 | log P (HCO₂H) = 4.80; LC-MS: m/z = 418 [M + H]^{+c)} |
| I-230 | δ (DMSO-d₆) = 0.84 (t, 3H), 1.08 (d, 3H), 1.20-1.35 (m, 5H), 1.70 (m, 1H), 3.63 (s, 3H), |
| | 3.87 (m, 1H), 3.94 (s, 3H); LC-MS: m/z = 284 [M + H]⁺ |
| I-231 | δ = 3.85 (s, 3H), 5.21 (s, 2H), 6.41 (s, 1H), 7.26-7.35 (m, 3H), 7.40 (t, 2H), 7.50-7.56 (m, |
| | 5H); LC-MS: m/z = 352 [M + H]⁺ |
| I-232 | δ = 1.35 (s, 9H), 3.85 (s, 3H), 5.24 (s, 2H), 6.49 (br. s, 1H), 7.30 (t, 1H), 7.34 (t, 2H), |
| | 7.44 (d, 2H), 7.50-7.53 (m, 4H)^{b)}; LC-MS: m/z = 408 [M + H]⁺ |
| I-233 | δ = 3.87 (s, 6H), 5.23 (s, 2H), 6.44 (s, 1H), 6.93 (d, 2H), 7.26-7.35 (m, 3H), 7.51 (m, 2H), |
| | 7.57 (d, 2H); LC-MS: m/z = 382 [M + H]⁺ |
| I-234 | δ = 3.79 (s, 3H), 3.88 (2s, 6H), 5.17 (s, 2H), 6.43 (s, 1H), 6.83 (d, 2H), 6.91 (d, 2H), 7.48 (d, |
| | 2H), 7.54 (d, 2H); LC-MS: m/z = 412 [M + H]⁺ |
| I-235 | δ = 3.78 (s, 3H), 3.80 (s, 3H), 3.86 (s, 3H), 4.15 (s, 3H), 5.10 (s, 2H), 6.82 (d, 2H), 6.90 (d, |
| | 2H), 7.25 (d, 2H), 7.63 (d, 2H); LC-MS: m/z = 426 [M + H]⁺ |
| I-236 | δ = 3.86 (s, 3H), 5.24 (s, 2H), 6.45 (br. s, 1H), 6.98 (d, 2H), 7.08 (d, 2H), 7.19 (t, 1H), |
| | 7.28-7.35 (m, 3H), 7.39 (m, 2H), 7.50-7.56 (m, 4H); LC-MS: m/z = 444 [M + H]⁺ |
| I-237 | δ = 3.82 (s, 3H), 4.10 (s, 3H), 5.16 (s, 2H), 7.21-7.25 (m, 3H), 7.30 (t, 2H), 7.40 (t, 2H), |
| | 7.47 (t, 1H), 7.58 (m, 2H); LC-MS: m/z = 366 [M + H]⁺ |
| I-238 | δ = 3.77 (s, 3H), 3.84 (s, 3H), 4.02 (s, 3H), 5.21 (s, 2H), 6.83 (d, 2H), 7.43 (d, 2H); LC-MS: |
| | m/z = 336 [M + H]⁺ |
| I-239 | δ = 0.82 (t, 3H), 0.95-1.75 (m, 13H), 3.83 (s, 3H), 4.11 (s, 3H); LC-MS: m/z = 324 [M + H]⁺ |
| I-240 | δ (DMSO-d₆) = 1.00-1.10 (m, 2H), 1.07 (d, 3H), 1.21-1.35 (m, 3H), 1.41-1.59 (m, 4H), |
| | 1.65-1.75 (m, 4H), 3.61 (s, 3H), 3.81 (m, 1H), 3.93 (s, 3H); LC-MS: m/z = 324 [M + H]⁺ |
| I-241 | δ (DMSO-d₆) = 0.80-0.85 (m, 9H), 1.08-1.14 (m, 2H), 1.35-1.51 (m, 3H), |
| | 1.61-1.68 (m, 2H), 3.69 (s, 3H), 3.72 (s, 3H), 4.14 (s, 3H), 5.01-5.07 (m, 2H), 6.87 (d, 2H), 7.19 (d, |
| | 2H); LC-MS: m/z = 432 [M + H]⁺ |
| I-242 | δ (DMSO-d₆) = 0.84 (t, 3H), 0.97 (t, 3H), 1.47 (m, 1H), 1.61-1.68 (m, 2H), 1.91 (m, 1H), |
| | 3.20-3.32 (m, 4H), 3.68 (s, 3H), 3.72 (s, 3H), 4.13 (s, 3H), 4.22 (m, 1H), 5.00-5.09 (m, |
| | 2H), 6.88, (d, 2H), 7.20 (d, 2H); LC-MS: m/z = 434 [M + H]⁺ |
| I-243 | δ = 3.79 (s, 3H), 3.80 (s, 3H), 7.38-7.48 (m, 3H), 7.60-7.68 (m, 6H); LC-MS: m/z = 352 [M + H]⁺ |
| I-244 | LC-MS: m/z = 453 [M + H]^{+c)} |
| I-245 | δ (DMSO-d₆) = 3.57 (s, 3H), 3.80 (s, 3H); LC-MS: m/z = 216 [M + H]⁺ |
| I-246 | log P (H₃PO₄) = 4.82; LC-MS: m/z = 425 [M + H]^{+c)} |
| I-247 | δ = 1.90-2.30 (m, 2H), 2.85-3.09 (m, 4H), 3.78 (s, 3H), 3.89 (s, 3H), 5.14 (s, 2H), |
| | 6.83 (d, 2H), 7.09-7.15 (m, 4H), 7.45 (d, 2H); LC-MS: m/z = 436 [M + H]⁺ |
| I-248 | δ (MeCN-d³) = 0.87 (t, 3H), 1.26 (m, 2H), 1.53 (m, 2H), 2.91 (s, 3H), 3.33 (t, 2H), 3.69 (s, |
| | 3H), 3.87 (s, 3H); LC-MS: m/z = 285 [M + H]⁺ |
| I-249 | δ^{b)} = 3.79 (s, 3H), 3.85 (s, 3H), 5.18 (s, 2H), 6.82 (d, 2H), 7.20 (d, 2H), 7.39 (d, 2H), 7.45 (d, |
| | 2H); LC-MS: m/z = 416 {[M(Cl³⁵) + H]⁺, 1 Cl} |
| I-250 | δ = 0.80-0.85 (m, 6H), 1.18 (d, 3H), 1.25-1.35 (m, 6H), 1.74 (m, 1H), 3.77 (s, 3H), |
| | 3.88 (s, 3H), 5.08-5.18 (m, 2H), 6.82 (d, 2H), 7.44 (d, 2H); LC-MS: m/z = 418 [M + H]⁺ |
| I-251 | δ = 1.65 (d, 3H), 2.35-2.39 (m, 2H), 3.03 (t, 3H), 3.77 (s, 3H), 3.89 (s, 3H), 4.13 (q, 1H), |
| | 5.11 (s, 2H), 5.43-5.52 (m, 2H), 6.81 (d, 2H), 7.42 (d, 2H); LC-MS: m/z = 374 [M + H]⁺ |
| I-252 | δ = 1.21-1.82 (m, 10H), 3.57 (m, 1H), 3.77 (s, 3H), 3.84 (s, 3H), 3.91 (s, 3H), 5.17 (s, 2H), |
| | 6.38 (dd, 1H), 6.45 (d, 1H), 6.84 (d, 1H); LC-MS: m/z = 416 [M − H]⁻ |
| I-253 | δ (DMSO-d₆) = 2.33 (s, 3H), 3.76 (s, 3H), 4.14 (s, 3H), 7.14 (d, 2H), 7.47 (d, 2H); LC-MS: |
| | m/z = 305 [M + H]⁺ |
| I-254 | LC-MS: m/z = 268 [M + H]^{+c)} |
| I-255 | δ = 3.78 (2s, 6H), 3.96 (s, 3H), 5.20 (s, 2H), 6.58 (s, 1H), 6.84 (d, 2H), 7.25 (d, 2H); LC- |
| | MS: m/z = 292 [M + H]⁺ |
| I-256 | LC-MS: m/z = 416 [M − H]^{-c)} |
| I-257 | δ = 1.18 (d, 3H), 2.81 (dd, 1H), 3.06 (dd, 1H), 3.75 (s, 3H), 3.78 (s, 3H), 4.11 (s, 3H), |
| | 4.52 (m, 1H), 5.09-5.16 (m, 2H), 6.83 (d, 2H), 6.95-7.05 (m, 2H), 7.15 (m, 1H), 7.26 (d, 2H), |
| | 7.32 (m, 1H); LC-MS: m/z = 456 [M + H]⁺ |
| I-258 | δ = 1.14 (d, 3H), 1.77 (s, 3H), 2.04 (m, 1H), 2.54 (m, 1H), 3.75 (s, 3H), 3.79 (s, 3H), 4.10 (s, |
| | 3H), 4.46 (m, 1H), 4.71 (s, 1H), 4.75 (s, 1H), 5.14 (s, 2H), 6.84 (d, 2H), 7.25 (d, 2H); LC- |
| | MS: m/z = 402 [M + H]⁺ |
| I-259 | LC-MS: m/z = 506 [M + H]^{+c)} |
| I-260 | LC-MS: m/z = 594 {[M(Cl³⁵) + H]⁺, 1 Cl}^{c)} |
| I-261 | δ (DMSO-d₆) = 1.06 (t, 3H), 1.25 (t, 3H), 3.09 (q, 2H), 3.67 (s, 3H), 3.72 (s, 3H), 4.48 (q, |
| | 2H), 5.06 (s, 2H), 6.88 (d, 2H), 7.18 (d, 2H); LC-MS: m/z = 362 [M + H]⁺ |
| I-262 | δ = 1.13 (d, 3H), 1.29 (s, 9H), 2.53 (dd, 1H), 3.12 (dd, 1H), 3.75 (s, 3H), 3.78 (s, 3H), |
| | 4.11 (s, 3H), 4.50 (m, 1H), 5.14 (s, 2H), 6.84 (d, 2H), 7.20-7.30 (m, 6H); LC-MS: m/z = 494 [M + H]⁺ |
| I-263 | LC-MS: m/z = 472 [M + H]^{+c)} |
| I-264 | LC-MS: m/z = 454 [M + H]^{+c)} |
| I-265 | LC-MS: m/z = 486 [M + H]^{+c)} |
| I-266 | LC-MS: m/z = 468 [M + H]^{+c)} |
| I-267 | δ = 1.12 (2d, 3H, 2 diastereomers), 1.35-2.00 (m, 6H), 2.56, 2.65 (2m, 1H, 2 |
| | diastereomers) 3.76 (2s, 3H, 2 diastereomers), 3.78 (s, 3H), 4.10 (s, 3H), 4.20-4.28 (m, |
| | 1H), 5.05-5.20 (m, 2H), 5.53-5.85 (m, 2H), 6.84 (d, 2H), 7.23 (d, 2H); LC-MS: m/z = 428 [M + H]⁺ |
| I-268 | δ = 1.21 (d, 3H), 2.29 (m, 1H), 2.69 (m, 1H), 3.75 (s, 3H), 3.78 (s, 3H), 4.11 (s, 3H), |
| | 4.35 (m, 1H), 5.13 (s, 2H), 6.25 (m, 1H), 6.40 (d, 1H), 6.84 (d, 2H), 7.18 (t, 1H), 7.24-7.35 (m, |
| | 6H); LC-MS: m/z = 484 [M + H]⁺ |
| I-269 | δ = 1.12 (d, 3H), 1.42 (m, 1H), 1.68-2.15 (m, 10H), 2.45 (m 1H), 3.75 (s, 3H), 3.78 (s, |
| | 3H), 4.10 (s, 3H), 4.42 (m, 1H), 4.65-4.71 (m, 2H), 5.14 (s, 2H), 5.44 (m, 1H), 6.84 (d, |
| | 2H), 7.25 (d, 2H); LC-MS: m/z = 482 [M + H]⁺ |
| I-270 | δ = 1.15 (d, 3H), 2.01 (m, 1H), 2.51 (m, 1H), 3.48 (s, 2H), 3.75 (s, 3H), 3.78 (s, 3H), 4.11 (s, |
| | 3H), 4.55 (m, 1H), 4.75 (s, 1H), 4.85 (s, 1H), 5.11-5.17 (m, 2H), 6.84 (d, 2H), |
| | 7.10-7.30 (m, 7H); LC-MS: m/z = 478 [M + H]⁺ |
| I-271 | LC-MS: m/z = 472 [M + H]^{+c)} |
| I-272 | δ = 1.25 (t, 3H), 3.05 (q, 2H), 3.88 (s, 3H), 4.62 (d, 2H), 5.13-5.25 (m, 2H), 5.89 (m, 1H); |
| | LC-MS: m/z = 254 [M + H]⁺ |
| I-273 | δ = 1.14 (d, 3H), 2.54 (dd, 1H), 3.13 (dd, 1H), 3.75 (s, 3H), 3.78 (s, 3H), 4.12 (s, 3H), |
| | 4.49 (m, 1H), 5.14 (s, 2H), 6.84 (d, 2H), 6.87-6.93 (m, 4H), 7.21-7.27 (m, 4H); LC-MS: m/z = 564 {[M(Cl³⁵) + H]⁺, |
| | 1 Cl} |
| I-274 | LC-MS: m/z = 445 {[M(Cl³⁵) + H]⁺, 1 Cl}^{c)} |
| I-275 | δ = 1.13 (d, 3H), 2.55 (dd, 1H), 3.11 (dd, 1H), 3.75 (s, 3H), 3.80 (s, 3H), 4.10 (s, 3H), |
| | 4.49 (m, 1H), 5.13 (s, 2H), 6.85 (d, 2H), 7.21-7.26 (m, 6H); LC-MS: m/z = 470 {[M(Cl³⁵) − H]⁻, |
| | 1 Cl} |
| I-276 | δ = 1.17 (d, 3H), 2.35 (s, 3H), 2.67 (dd, 1H), 3.10 (dd, 1H), 3.74 (s, 3H), 3.79 (s, 3H), |
| | 4.10 (s, 3H), 4.63 (m, 1H), 5.12 (s, 2H), 6.83 (d, 2H), 7.06-7.13 (m, 3H), 7.22-7.26 (m, 3H); |
| | LC-MS: m/z = 452 [M + H]⁺ |
| I-277 | δ = 1.12 (d, 3H), 2.31 (s, 3H), 2.54 (dd, 1H), 3.11 (dd, 1H), 3.75 (s, 3H), 3.78 (s, 3H), |
| | 4.10 (s, 3H), 4.50 (m, 1H), 5.14 (s, 2H), 6.83 (d, 2H), 7.06 (d, 2H), 7.17 (d, 2H), 7.25 (d, 2H); |
| | LC-MS: m/z = 452 [M + H]⁺ |
| I-278 | δ = 1.13 (d, 3H), 2.54 (m, 1H), 3.10 (m, 1H), 3.81 (s, 3H), 4.08 (s, 3H), 4.19 (m, 1H), |
| | 7.07-7.13 (m, 4H); LC-MS: m/z = 332 [M + H]⁺ |
| I-279 | LC-MS: m/z = 430 [M + H]^{+c)} |
| I-280 | LC-MS: m/z = 404 [M + H]^{+c)} |
| I-281 | LC-MS: m/z = 404 [M + H]^{+c)} |
| I-282 | LC-MS: m/z = 460 [M + H]^{+c)} |
| I-283 | LC-MS: m/z = 418 [M + H]^{+c)} |
| I-284 | δ = 1.55 (d, 3H) 3.78 (s, 3H), 3.89 (s, 3H), 5.10 (s, 2H), 6.82 (d, 2H), 6.88-6.95 (m, 4H), |
| | 7.27 (d, 2H), 7.32 (d, 2H), 7.41 (d, 2H); LC-MS: m/z = 536 {[M(Cl³⁵) + H]⁺, 1 Cl} |
| I-285 | δ = 0.92, 0.93 (2d, 3H, 2 diastereomers), 1.15-1.18 (m, 9H), 3.77 (s, 3H), 3.89 (s, 3H), |
| | 5.12 (s, 2H), 6.83 (d, 2H), 7.44 (d, 2H); LC-MS: m/z = 402 [M + H]⁺ |
| I-286 | δ = 1.27 (d, 3H), 2.97 (m, 1H), 3.21 (m, 1H), 3.74 (s, 3H), 3.78 (s, 3H), 4.12 (s, 3H), |
| | 4.48 (m, 1H), 5.13 (s, 2H), 6.82-6.87 (m, 3H), 7.10 (d, 1H), 7.25 (d, 2H); LC-MS: m/z = 478 {[M(Cl³⁵) + H]⁺, |
| | 1 Cl} |
| I-287 | LC-MS: m/z = 418 [M + H]^{+c)} |
| I-288 | LC-MS: m/z = 474 [M + H]^{+c)} |
| I-289 | LC-MS: m/z = {[M(Cl³⁵) + H]⁺, 2 Cl}^{c)} |
| I-290 | δ = 1.50 (d, 3H), 3.69 (s, 3H), 3.76 (s, 3H), 4.08 (q, 1H), 5.07 (s, 2H), 6.78-6.85 (m, 3H), |
| | 7.18-7.45 (m, 4H); LC-MS: m/z = 478 {[M(Cl³⁵) + H]⁺, 2 Cl} |
| I-291 | δ = 1.46 (d, 3H), 3.73 (s, 3H), 3.79 (s, 3H), 4.10 (s, 3H), 5.04 (d, 1H), 5.15 (d, 1H), 5.59 (q, |
| | 1H), 6.82 (d, 2H), 7.15 (d, 2H), 7.35 (d, 1H), 7.41 (d, 1H), 7.53 (d, 1H); LC-MS: m/z = 492 {[M(Cl³⁵) + H]⁺, |
| | 2 Cl} |
| I-292 | δ = 1.50 (d, 3H), 3.75 (s, 3H), 3.79 (s, 3H), 4.10 (s, 3H), 5.00 (d, 1H), 5.20 (d, 1H), 5.73 (q, |
| | 1H), 6.80 (d, 2H), 7.09 (d, 2H), 7.16-7.22 (m, 2H), 7.40 (d, 1H); LC-MS: m/z = 492 {[M(Cl³⁵) + H]⁺, |
| | 2 Cl} |
| I-293 | LC-MS: m/z = 443 [M + H]^{+c)} |
| I-294 | LC-MS: m/z = 457 [M + H]^{+c)} |
| I-295 | LC-MS: m/z = 450 [M + H]^{+c)} |
| I-296 | LC-MS: m/z = 404 [M + H]^{+c)} |
| I-297 | δ = 3.79 (s, 3H), 3.85 (s, 3H), 5.20 (s, 2H), 6.87 (d, 2H), 7.50-8.15 (m, 9H); LC-MS: m/z = 432 [M + H]⁺ |
| I-298 | LC-MS: m/z = 400 [M + H]^{+c)} |
| I-299 | LC-MS: m/z = 458 [M + H]^{+c)} |
| I-300 | LC-MS: m/z = 450 [M + H]^{+c)} |
| I-301 | δ = 1.15 (d, 3H), 1.30 (t, 3H), 1.61 (s, 3H), 1.67 (s, 3H), 2.15 (m, 1H), 2.40 (m, 1H), 3.74 (s, |
| | 3H), 3.78 (s, 3H), 4.25 (m, 1H), 4.47 (q, 2H), 5.14-5.18 (m, 3H), 6.83 (d, 2H), 7.23 (d, |
| | 2H); LC-MS: m/z = 430 [M + H]⁺ |
| I-302 | δ = 1.51 (d, 3H), 3.71 (s, 3H), 3.78 (s, 3H), 4.05 (s, 3H), 5.11 (s, 2H), 5.66 (q, 1H), 6.80 (d, |
| | 2H), 7.13 (d, 2H), 7.20-7.31 (m, 3H), 7.43 (d, 2H); LC-MS: m/z = 424 [M + H]⁺ |
| I-303 | δ = 0.83-0.92 (m, 6H), 1.08-1.19 (m, 5H), 1.30-1.41 (m, 2H), 1.59, 1.88 (2m, 1H, 2 |
| | diastereomers), 3.75 (s, 3H), 3.78 (s, 3H), 4.09 (2m, 3H, 2 diastereomers), 4.31-4.43 (m, |
| | 1H, 2 diastereomers), 5.14 (s, 2H), 6.83 (d, 2H), 7.24 (d, 2H); LC-MS: m/z = 418 [M + H]⁺ |
| I-304 | LC-MS: m/z = 422 {[M(Cl³⁵) + H]⁺, 1 Cl}^{c)} |
| I-305 | LC-MS: m/z = 464 [M + H]^{+c)} |
| I-306 | LC-MS: m/z = 446 [M + H]^{+c)} |
| I-307 | LC-MS: m/z = 414 [M + H]^{+c)} |
| I-308 | LC-MS: m/z = 472 [M + H]^{+c)} |
| I-309 | δ (MeCN-d³) = 0.87 (d, 3H), 0.88 (d, 3H), 1.10 (d, 3H), 1.18 (m, 1H), 1.37 (t, 3H), 1.59 (m, |
| | 1H), 1.68 (m, 1H), 3.70 (s, 3H), 4.08 (m, 1H), 4.43 (q, 2H); LC-MS: m/z = 298 [M + H]⁺ |
| I-310 | δ = 1.00-1.11 (m, 2H), 1.19 (d, 3H), 1.25-1.80 (m, 11H), 3.77 (s, 3H), 3.87 (s, 3H), |
| | 5.15 (s, 2H), 6.83 (d, 2H), 7.44 (d, 2H); LC-MS: m/z = 430 [M + H]⁺ |
| I-311 | δ = 1.60-1.96 (m, 8H), 3.77 (s, 3H), 3.87 (s, 3H), 3.98 (m, 1H); 5.13 (s, 2H), 6.82 (d, 2H), |
| | 7.44 (d, 2H); LC-MS: m/z = 374 [M + H]⁺ |
| I-312 | δ = 0.87 (m, 3H, 2 diastereomers), 1.30-2.00 (m, 8H), 2.46-2.68 (2m, 1H, 2 |
| | diastereomers), 3.76 (s, 3H), 3.79 (s, 3H), 4.09 (s, 3H), 4.25-4.38 (2m, 1H, 2 |
| | diastereomers); 5.09-5.18 (m, 2H), 5.56-5.75 (m, 2H), 6.84 (d, 2H), 7.23 (d, 2H); LC- |
| | MS: m/z = 442 [M + H]⁺ |
| I-313 | δ = 0.93 (t, 3H), 1.55 (m, 1H), 1.74 (m, 1H), 1.76 (s, 3H), 2.11 (m, 1H), 2.50 (m, 1H), |
| | 3.75 (s, 3H), 3.79 (s, 3H), 4.09 (s, 3H), 4.54 (m, 1H); 4.68-4.72 (m, 2H), 5.10-5.18 (m, 2H), |
| | 6.84 (d, 2H), 7.24 (d, 2H); LC-MS: m/z = 416 [M + H]⁺ |
| I-314 | δ (MeCN-d³) = 0.88 (t, 3H), 1.27 (m, 2H), 1.55 (m, 2H), 2.92 (s, 3H), 3.37 (t, 2H), 3.68 (s, |
| | 3H), 3.74 (s, 3H), 3.99 (s, 3H), 5.08 (s, 2H), 6.85 (d, 2H), 7.21 (d, 2H); LC-MS: m/z = 405 [M + H]⁺ |
| I-315 | δ = (DMF-d⁷) = 3.82 (s, 3H), 4.02 (s, 3H), 4.61 (d, 2H), 5.25 (s, 2H), 7.21-7.38 (m, 10H), |
| | 10.52 (br. t, 1H), 16.61 (br. s, 1H) |
| II-1 | δ = 1.11 (d, 3H), 1.43 (m, 1H), 1.50-1.60 (m, 2H), 1.77 (m, 1H), 2.28 (s, 3H), 2.31 (s, 3H), |
| | 2.92 (m, 1H), 3.30 (s, 3H), 3.34 (t, 2H), 3.84 (s, 3H), 3.99 (s, 3H); LC-MS: m/z = 384 [M + H]⁺ |

^{a)}Unless indicated otherwise, ¹H-NMR spectra were recorded in CDCl₃ at 400 MHz.
^{b)}The ¹H-NMR spectra were recorded in CDCl₃ at 700 MHz.
^{c)}Product reacted further (after having been checked by LC-MS).

The stated log P values were determined in accordance with EEC Directive 79/831 Annex V.A8 by HPLC (High Performance Liquid Chromatography) on a reversed-phase column (C18).

HP1100 LC System (UV/DAD); Kromasil 100 C18, 3.5 μm, 75×2.0 mm; mobile phase A: acetonitrile (0.1% formic acid); mobile phase B: water (0.09% formic acid); linear gradient from 10% acetonitrile to 95% acetonitrile over 8.5 min, then 95% acetonitrile for a further 3.5 min; oven temperature 43° C.; flow rate: 0.5 ml/min

HP1100 LC System (UV/DAD); Kromasil 100 C18, 3.5 μm, 70×3.1 mm; mobile phase A: acetonitrile (0.1% phosphoric acid); mobile phase B: water (0.1% phosphoric acid); linear gradient from 10% acetonitrile to 95% acetonitrile over 8.5 min, then 95% acetonitrile for a further 3 min; oven temperature 43° C.; flow rate: 1 ml/min

### USE EXAMPLES

### Example A

### Phytophthora Test (Tomato)/Protective

Solvent: 49 parts by weight of N,N-dimethylformamide

Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration. To test for protective activity, young tomato plants are sprayed with the preparation of active compound at the stated application rate. One day after the treatment, the plants are inoculated with a spore suspension of Phytophthora infestans and then remain at 100% relative humidity and 22° C. for 24 h. The plants are then placed in a climatized cell at about 96% relative atmospheric humidity and a temperature of about 20° C. Evaluation is carried out 7 days after the inoculation. 0% means an efficacy which corresponds to that of the control, whereas an efficacy of 100% means that no infection is observed.

TABLE A
Phytophthora test (tomato)/protective

| | | | |
|---|---|---|---|
| | | Active compound | |
| | | application rate | |
| Ex. | Active compound | in ppm | Efficacy in % |

Known:

| | | | |
|---|---|---|---|
| I-1 | 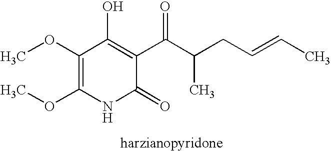 | 500 | 0 |

According to the invention:

| | | | |
|---|---|---|---|
| I-28 | 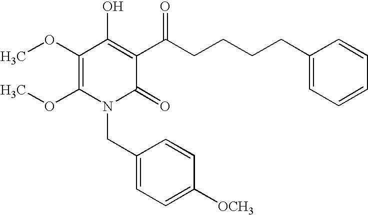 | 500 | 50 |
| I-48 | 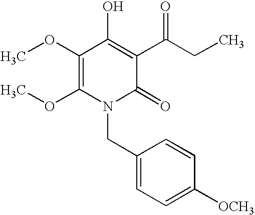 | 500 | 63 |
| I-49 | 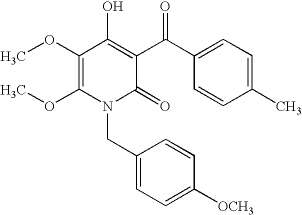 | 500 | 50 |
| I-55 | 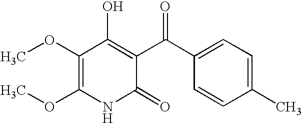 | 500 | 56 |
| I-69 | 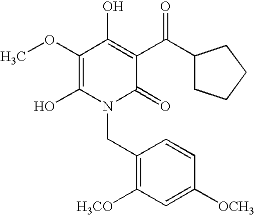 | 500 | 50 |
| I-89 | 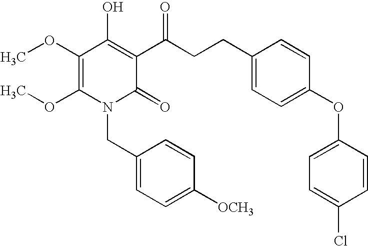 | 500 | 50 |
| I-101 | 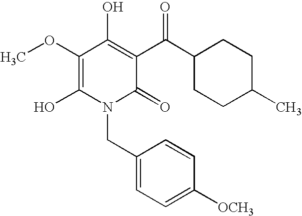 | 500 | 78 |
| I-107 | 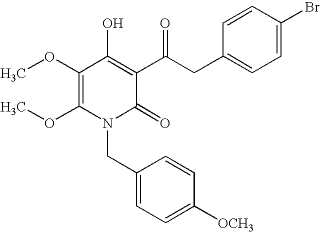 | 500 | 78 |
| I-110 | 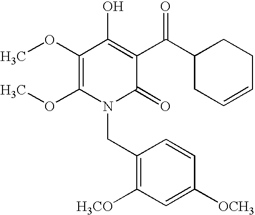 | 500 | 78 |
| I-111 | 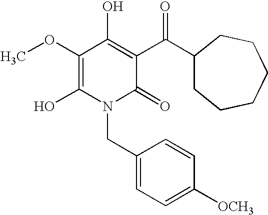 | 500 | 89 |
| I-122 | 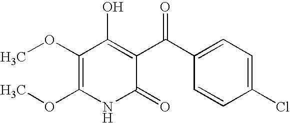 | 500 | 56 |
| I-129 | 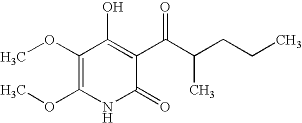 | 500 | 56 |
| I-130 | 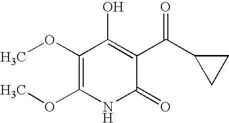 | 500 | 56 |
| I-139 | 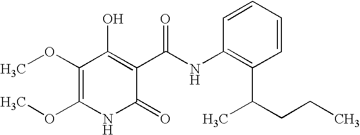 | 500 | 60 |
| I-141 | 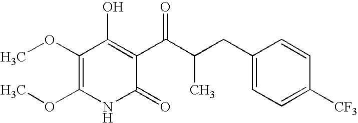 | 500 | 70 |
| I-158 | 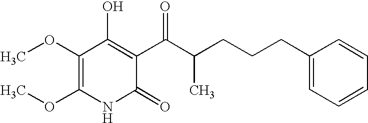 | 500 | 60 |
| I-172 | 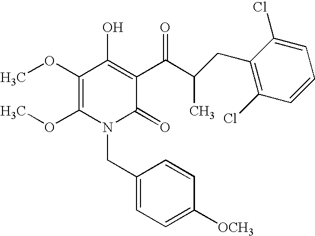 | 500 | 71 |
| I-178 | 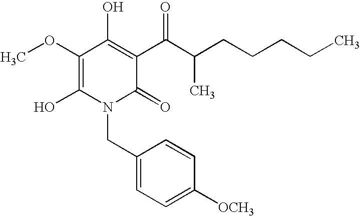 | 500 | 60 |
| I-180 | 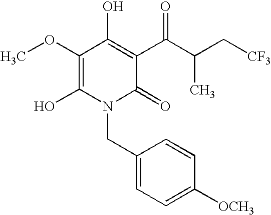 | 500 | 50 |
| I-221 | 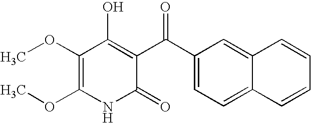 | 500 | 80 |

### Example B

### Alternaria Test (Tomato)/Protective

Solvent: 49 parts by weight of N,N-dimethylformamide

Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration. To test for protective activity, young tomato plants are sprayed with the preparation of active compound at the stated application rate. One day after the treatment, the plants are inoculated with a spore suspension of Alternaria solani and then remain at 100% relative humidity and 22° C. for 24 h. The plants then remain at 96% relative atmospheric humidity and a temperature of 20° C. Evaluation is carried out 7 days after the inoculation. 0% means an efficacy which corresponds to that of the control, whereas an efficacy of 100% means that no infection is observed.

TABLE B
Alternaria test (tomato)/protective

| | | | |
|---|---|---|---|
| | | Active compound | |
| | | application rate | |
| Ex. | Active compound | in ppm g/ha | Efficacy in % |

Known:

| | | | |
|---|---|---|---|
| I-1 | 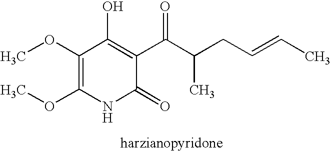 | 500 | 0 |

According to the invention:

| | | | |
|---|---|---|---|
| I-48 | 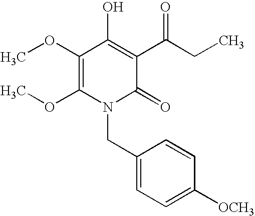 | 500 | 57 |
| I-66 | 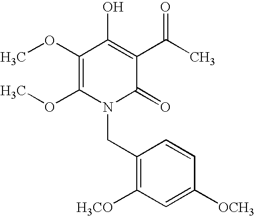 | 500 | 94 |
| I-72 | 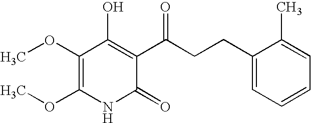 | 500 | 50 |
| I-75 | 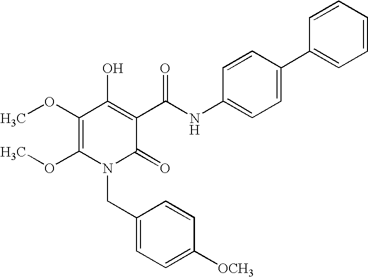 | 500 | 70 |
| I-108 | 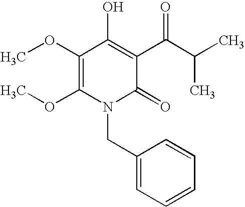 | 500 | 67 |
| I-121 | 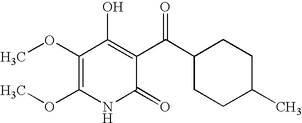 | 500 | 56 |
| I-141 | 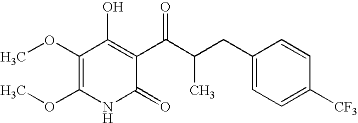 | 500 | 80 |
| I-149 | 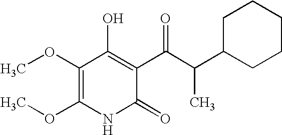 | 500 | 80 |
| I-158 | 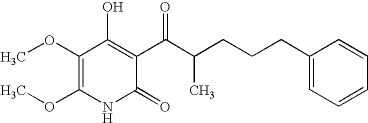 | 500 | 50 |
| I-153 | 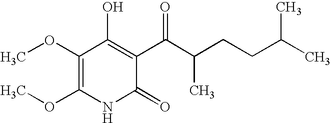 | 500 | 90 |
| I-159 | 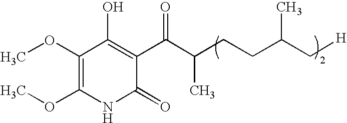 | 500 | 95 |
| I-157 | 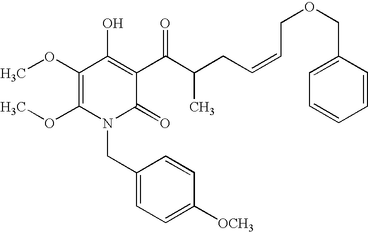 | 500 | 50 |
| I-160 | 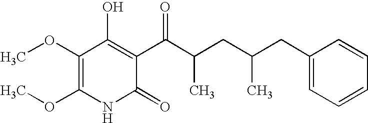 | 500 | 70 |
| I-164 | 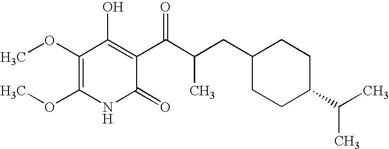 | 500 | 90 |
| I-230 | 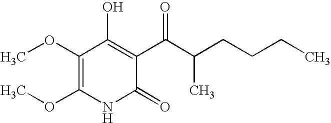 | 500 | 70 |
| I-184 | 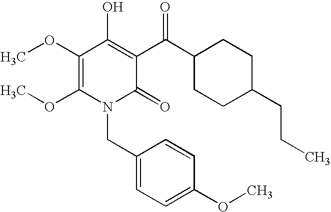 | 500 | 56 |
| I-212 | 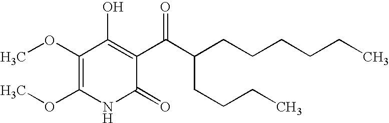 | 500 | 71 |
| I-221 | 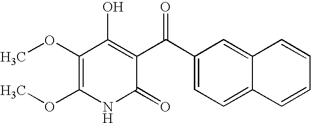 | 500 | 93 |
| I-219 | 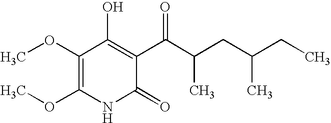 | 500 | 50 |
| I-228 | 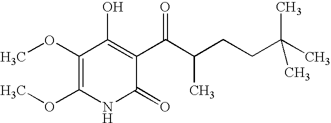 | 500 | 80 |

### Example C

### Sphaerotheca Test (Cucumber)/Protective

Solvents: 24.5 parts of acetone
24.5 parts of weight of dimethylacetamide

Emulsifier: 1 part by weight of alkylaryl polyglycol ether

To produce a suitable preparation of active compound, 1 part by weight of active compound is mixed with the stated amounts of solvents and emulsifier, and the concentrate is diluted with water to the desired concentration. To test for protective activity, young plants are sprayed with the preparation of active compound at the stated application rate. After the spray coating has dried on, the plants are inoculated with an aqueous spore suspension of Sphaerotheca fuliginea. The plants are then placed in a greenhouse at about 23° C. and a relative atmospheric humidity of about 70%. Evaluation is carried out 7 days after the inoculation. 0% means an efficacy which corresponds to that of the control, whereas an efficacy of 100% means that no infection is observed.

TABLE C
Sphaerotheca test (cucumber)/protective

| | | | |
|---|---|---|---|
| | | Active compound | |
| | | application rate | |
| Ex. | Active compound | in g/ha | Efficacy in % |

Known:

| | | | |
|---|---|---|---|
| I-1 | 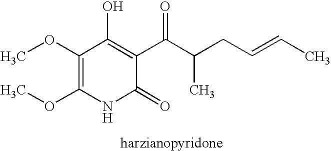 | 100 | 77 |

According to the invention:

| | | | |
|---|---|---|---|
| I-129 | 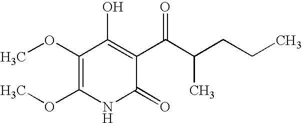 | 100 | 98 |
| I-138 | 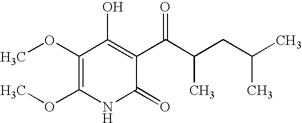 | 100 | 96 |
| I-230 | 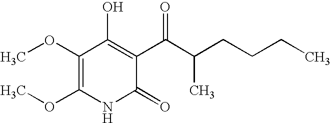 | 100 | 98 |
| I-197 | 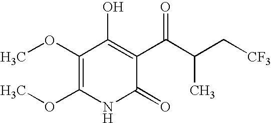 | 100 | 100 |
| I-204 | 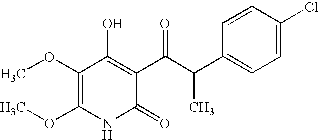 | 100 | 85 |
| I-240 | 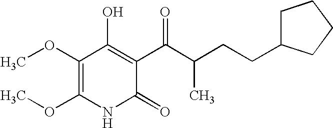 | 100 | 98 |
| I-74 | 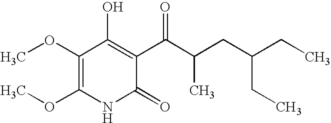 | 100 | 100 |

### Example D

### Fusarium graminearum Test (Barley)/Curative

Solvent: 50 parts by weight of N,N-dimethylacetamide

Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration. To test for curative activity, young plants are sprayed with a spore suspension of Fusarium graminearum. The plants remain in an incubation cabin at 10° C. and 100% relative atmospheric humidity for 24 hours and are then sprayed with the preparation of active compound at the stated application rate. After the spray coating has dried on, the plants remain in a greenhouse under a translucent incubation hood at a temperature of about 10° C. and a relative atmospheric humidity of about 100%. Evaluation is carried out 5 days after the inoculation. 0% means an efficacy which corresponds to that of the control, whereas an efficacy of 100% means that no infection is observed. In this test, the following compounds according to the invention show, at an active compound concentration of 1000 ppm, an efficacy of 70% or more: I-196, I-197, I-200, I-201, I-204, I-208, I-211.

1. Process for preparing 2-pyridones of the formula (I) 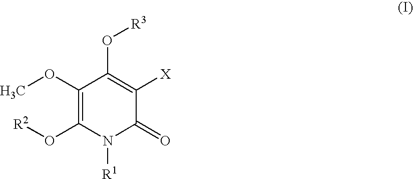
in which
X represents hydrogen, —C(═O)OR⁵, —C(═O)NR⁶R⁷, —C(═O)CR⁸R⁹R¹⁰, or represents —C(═O)aryl or —(C═O)hetaryl, each of which is optionally substituted in the aryl or heteraryl moiety,
R¹ represents hydrogen or alkyl, alkenyl, alkynyl, cycloalkyl, aryl, hetaryl, optionally mono- or polysubstituted by identical or different substituents,
R² represents hydrogen, methyl or ethyl,
R³ represents hydrogen,
R⁵ represents hydrogen or alkyl, alkenyl, alkynyl, cycloalkyl, cycloalkenyl, bicycloalkyl, aryl, hetaryl, optionally mono- or polysubstituted by identical or different substituents,
R⁶ represents hydrogen, C₁-C₈-alkyl, C₁-C₄-alkoxy-C₁-C₄-alkyl, C₃-C₈-cycloalkyl; C₂-C₆-haloalkyl, halo-C₂-C₄-alkoxy-C₁-C₄-alkyl, C₃-C₈-halocycloalkyl having in each case 1 to 9 fluorine, chlorine and/or bromine atoms; (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₂-C₃-alkoxy)carbonyl-C₁-C₃-alkyl having in each case 1 to 13 fluorine, chlorine and/or bromine atoms; represents —CH₂—C≡C—R¹¹ or —CH₂—CH═CH—R¹¹,
R⁷ represents hydrogen or represents alkyl, alkenyl, alkynyl, cycloalkylalkyl, cycloalkenylalkyl, each of which is optionally mono- or polysubstituted by identical or different substituents, or represents -M-Q-Z,
R⁸ represents hydrogen, alkyl or haloalkyl,
R⁹ represents hydrogen, halogen or represents alkyl, alkenyl, alkynyl, alkoxy, alkylthio, cycloalkyl, cycloalkenyl, or bicycloalkyl, each of which is optionally mono- or polysubstituted by identical or different substituents,
R¹⁰ represents hydrogen or represents alkyl, alkenyl, alkynyl, cycloalkyl, cycloalkylalkyl, cycloalkenyl, cycloalkenylalkyl, bicycloalkyl, bicycloalkylalkyl, aryl, arylalkyl, hetaryl or hetarylalkyl, each of which is optionally mono- or polysubstituted by identical or different substituents,
R⁹ and R¹⁰ furthermore together with the carbon atom to which they are attached form a carbocycle or heterocycle which may be saturated or unsaturated and which may be fused with a further carbocycle and which may furthermore optionally be substituted,
R¹¹ represents hydrogen, C₁-C₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)carbonyl or cyano,
M represents in each case optionally substituted cycloalkylene, cycloalkenylene, bicycloalkylene, arylene or hetarylene,
Q represents a direct bond, C₁-C₄-alkylene, C₂-C₄-alkenylene, C₁-C₄-alkylenoxy, oxy-C₁-C₄-alkylene, O, S, SO, SO₂ or NR¹²,
Z represents hydrogen or represents Z¹, Z², Z³, Z⁴, Z⁵ or Z⁶, where Q does not represent O, S, SO, SO₂, NR¹² if Z represents hydrogen,
Z¹ represents phenyl which is optionally mono- to pentasubstituted by identical or different substituents,
Z² represents pyridinyl which is optionally mono- to trisubstituted by identical or different substituents,
Z³ represents cycloalkyl or bicycloalkyl, each of which is optionally mono- or polysubstituted by identical or different substituents from the group consisting of halogen, alkyl, cycloalkyl and/or —(CR¹³R¹⁴)ₘSiR¹⁵R¹⁶R¹⁷,
Z⁴ represents unsubstituted C₁-C₂₀-alkyl or represents C₁-Cₘ-alkyl which is mono- or polysubstituted by identical or different substituents from the group consisting of halogen, alkylthio, alkylsulphynyl, alkylsulphonyl, alkoxy, alkylamino, dialkylamino, haloalkylthio, haloalkylsulphynyl, haloalkylsulphonyl, haloalkoxy, haloalkylamino, halodialkylamino, —SiR¹⁵R¹⁶R¹⁷ and C₃-C₆-cycloalkyl, where the cycloalkyl moiety for its part may optionally be mono- or polysubstituted by identical or different substituents from the group consisting of halogen and C₁-C₄-alkyl,
Z⁵ represents C₂-C₂₀-alkenyl or C₂-C₂₀-alkynyl, each of which is optionally mono- or polysubstituted by identical or different substituents from the group consisting of halogen, alkylthio, alkylsulphynyl, alkylsulphonyl, alkoxy, alkylamino, dialkylamino, haloalkylthio, haloalkylsulphynyl, haloalkylsulphonyl, haloalkoxy, haloalkylamino, halodialkylamino, —SiR¹⁵R^{16‘R}¹⁷ and/or C₃-C₆-cycloalkyl, where the cycloalkyl moiety for its part may optionally be mono- or polysubstituted by identical or different substituents from the group consisting of halogen and C₁-C₄-alkyl,
Z⁶ represents a saturated or unsaturated 3- to 7-membered ring which is optionally mono- or polysubstituted and which contains a silicon atom as ring member, in which Q represents a direct bond or C₁-C₄-alkylene,
or
M-Q-Z together represent 1H-2,3-dihydroinden-4-yl, 1,3-dihydro-2-benzofuran-4-yl or 1,3-dihydro-2-benzothien-4-yl, each of which is optionally mono- to trisubstituted by methyl, or represent 1,2,3,4-tetrahydro-9-isopropyl-1,4-methanonaphthalen-5-yl,
R¹² represents hydrogen, C₁-C₈-alkyl, C₁-C₄-alkoxy-C₁-C₄-alkyl, C₁-C₄-alkylthio-C₁-C₄-alkyl, C₃-C₈-alkenyl, C₃-C₈-alkynyl, C₂-C₆-haloalkyl, C₃-C₆-haloalkenyl, C₃-C₆-halo-alkynyl or C₃-C₆-cycloalkyl,
R¹³ represents hydrogen or C₁-C₄-alkyl,
R¹⁴ represents hydrogen or C₁-C₄-alkyl,
m represents 0, 1, 2 or 3,
R¹⁵ and R¹⁶ independently of one another represent hydrogen, C₁-C₈-alkyl, C₁-C₈-alkoxy, C₁-C₄-alkoxy-C₁-C₄-alkyl, C₁-C₄-alkylthio-C₁-C₄-alkyl or C₁-C₆-haloalkyl,
R¹⁷ represents hydrogen, C₁-C₈-alkyl, C₁-C₈-alkoxy, C₁-C₄-alkoxy-C₁-C₄-alkyl, C₂-C₈-alkenyl, C₂-C₈-alkynyl, C₁-C₆-haloalkyl, C₂-C₆-haloalkenyl, C₂-C₆-haloalkynyl, C₃-C₆-cycloalkyl, or represents in each case optionally substituted phenyl or phenylalkyl,
characterized in that
according to process (1)
(a) oxymalonic acid derivatives of the formula (III) 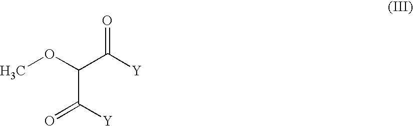
in which
Y represents halogen, hydroxyl or —O-AM in which
AM represents an alkali metal selected from the group consisting of lithium, sodium, potassium and caesium,
are reacted with amides of the formula (IV) 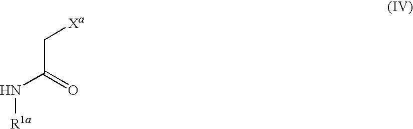
in which
R^{1a} represents alkyl, alkenyl, alkynyl, cycloalkyl, aryl, hetaryl, optionally mono- or polysubstituted by identical or different substituents,
X^{a} represents —C(═O)OR^{5a}, —C(═O)CR⁸R⁹R¹⁰ or represents —C(═O)aryl or —(C═O)hetaryl which is optionally substituted in the aryl or heteraryl moiety, and
R^{5a} represents alkyl, alkenyl, alkynyl, cycloalkyl, cycloalkenyl, bicycloalkyl, aryl, hetaryl, optionally mono- or polysubstituted by identical or different substituents, and
R⁸, R⁹ and R¹⁰ have the meanings given above,
if appropriate in the presence of a diluent,
thus giving 2-pyridones of the formula (I-b) 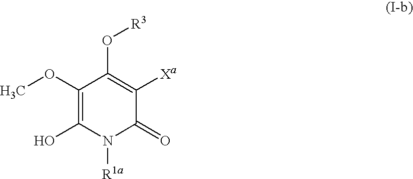
in which X^{a}, X^{1a} and R³ have the meanings given above
which are subsequently
(b) if appropriate reacted with an alkylating agent, if appropriate in the presence of a base, a Lewis acid or a molecular sieve and, if appropriate, in the presence of a diluent,
giving 2-pyridones of the formula (I-c) 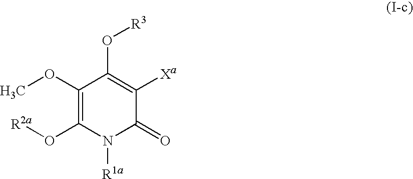
in which
X^{a}, R^{1a} and R³ have the meanings given above, and
R^{2a} represents methyl or ethyl,
after which
(c) if appropriate the 2-pyridones of the formula (I-b) or the formula (I-c) are hydrogenated in the presence of a transition metal catalyst or in the presence of Lewis or Brønsted acids, thus giving 2-pyridones of the formula (I-d) 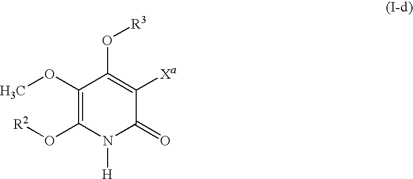
in which R², R³ and X^{a} have the meanings given above,
and
(d) if appropriate, the 2-pyridones of the formula (I-b) or the formula (I-c) or the formula (I-d), in which X^{a} represents —C(═O)OR^{5a} are cleaved, thus giving the 2-pyridones of the formula (I-e) 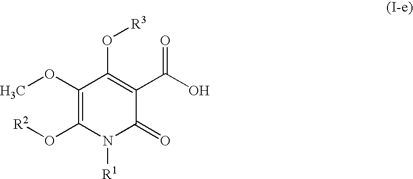
in which R¹, R² and R³ have the meanings given above;
or according to process (2)
(e) 2-pyridones of the formula (I-f) 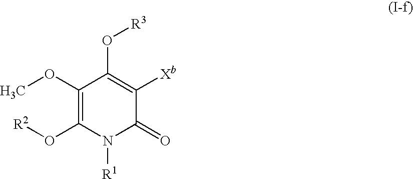
in which
R¹, R² and R³ have the meanings given above and
X^{b} represents —C(═O)OH or —C(═O)OR^{5b} in which
R^{5b} represents C₁-C₄-alkyl,
are reacted with amines of the formula (VIII) 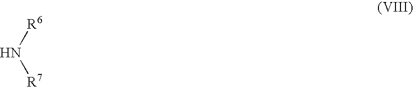
in which R⁶ and R⁷ have the meanings given above,
if appropriate in the presence of diluents and if appropriate in the presence of reaction auxiliaries, thus giving the 2-pyridones of the formula (I-g) 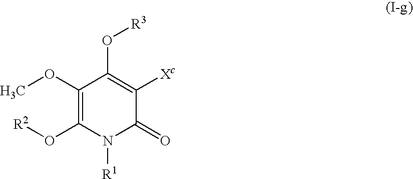
in which
R¹, R² and R³ have the meanings given above and
X^{c} represents —C(═O)NR⁶R⁷;
or
according to process (3)
(f) 2-pyridones of the formula (I-e) 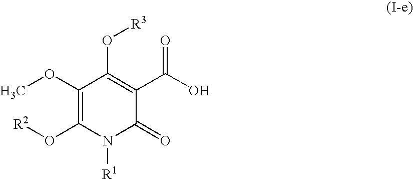
in which R¹, R² and R³ have the meanings given above
are reacted, if appropriate in the presence of diluents and if appropriate in the presence of basic reaction auxiliaries, thus giving the 2-pyridones of the formula (I-h) 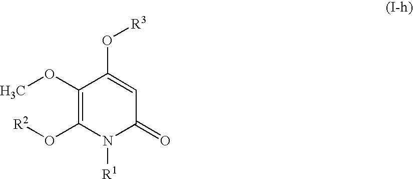
in which R¹, R² and R³ have the meanings given above;
and that 2-pyridinols of the formula (II) 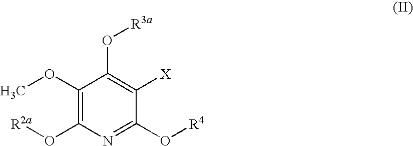
in which
X and R^{2a} have the meanings given above, and
R^{3a} and R⁴ each represent acetyl
are obtained by reacting,
according to process (4),
(g) 2-pyridones of the formula (I-i) 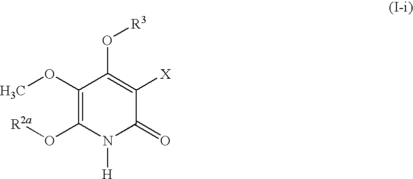
in which R^{2a}, R³ and X have the meanings given above
with an acetylating agent, if appropriate in the presence of a diluent and if appropriate in the presence of a basic reaction auxiliary.

2. Process for preparing 2-pyridones of the formula (I-b) 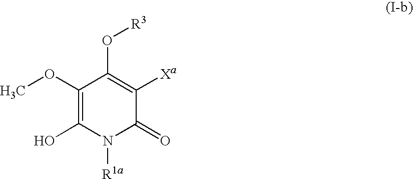
in which
X^{a} represents —C(═O)OR^{5a}, —C(═O)CR⁸R⁹R¹⁰ or represents —C(═O)aryl or —(C═O)hetaryl which is optionally substituted in the aryl or hetaryl moiety, and
R^{1a} represents alkyl, alkenyl, alkynyl, cycloalkyl, aryl, hetaryl, optionally mono- or polysubstituted by identical or different substituents,
R³ represents hydrogen,
R^{5a} represents alkyl, alkenyl, alkynyl, cycloalkyl, cycloalkenyl, bicycloalkyl, aryl, hetaryl, optionally mono- or polysubstituted by identical or different substituents, and
R⁸ represents hydrogen, alkyl or haloalkyl,
R⁹ represents hydrogen, halogen or represents alkyl, alkenyl, alkynyl, alkoxy, alkylthio, cycloalkyl, cycloalkenyl, or bicycloalkyl, each of which is optionally mono- or polysubstituted by identical or different substituents,
R¹⁰ represents hydrogen or represents alkyl, alkenyl, alkynyl, cycloalkyl, cycloalkylalkyl, cycloalkenyl, cycloalkenylalkyl, bicycloalkyl, bicycloalkylalkyl, aryl, arylalkyl, hetaryl or hetarylalkyl, each of which is optionally mono- or polysubstituted by identical or different substituents,
R⁹ and R¹⁰ furthermore together with the carbon atom to which they are attached form a carbocycle or heterocycle which may be saturated or unsaturated and which may be fused with a further carbocycle and which may furthermore optionally be substituted,
characterized in that
according to process (1)
(a) oxymalonic acid derivatives of the formula (III) 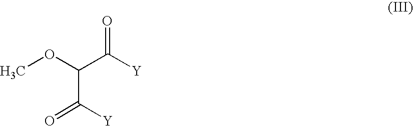
in which
Y represents halogen, hydroxyl or —O-AM in which
AM represents an alkali metal selected from the group consisting of lithium, sodium, potassium and caesium
are reacted with amides of the formula (IV) 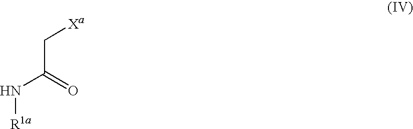
in which R^{1a} and X^{a} have the meanings given above,
if appropriate in the presence of a diluent.

3. 2-pyridones of the general formula (I-k) 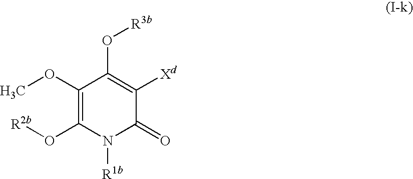
and 2-pyridinols of the general formula (II-b) 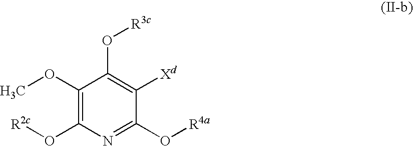
in which
X^{d} represents hydrogen, —C(═O)OR⁵, —C(═O)NR⁶R⁷, —C(═O)CR⁸R⁹R¹⁰, or represents —C(═O)aryl or —(C═O)hetaryl, each of which is optionally substituted in the aryl or heteraryl moiety,
R^{1b} represents hydrogen or alkyl, alkenyl, alkynyl, cycloalkyl, aryl, hetaryl, optionally mono- or polysubstituted by identical or different substituents,
R^{2b} represents hydrogen, methyl or ethyl,
R^{2c} represents methyl or ethyl,
R^{3b} represents hydrogen,
R^{3c} and R^{4a} both simultaneously represent hydrogen or both simultaneously represent acetyl,
and R⁵, R⁶, R⁷, R⁸, R⁹ and R¹⁰ have the meanings given in claim 1,
except for compounds in which
(1) X^{d} represents —C(═O)CR⁸R⁹R¹⁰,
R^{1b}, R^{3b} and R⁸ each represent hydrogen,
R^{2b}, R^{2c} and R⁹ each represent methyl,
R^{3c} and R^{4a} each represent hydrogen or acetyl and
R¹⁰ represents alkyl or alkenyl, each of which is optionally mono- or polysubstituted by identical or different halogens, or represents phenyl or represents 4-tert-butylbenzyl;
(2) X^{d} represents —C(═O)CR⁸R⁹R¹⁰,
R^{1b}, R^{3b} and R⁸ each represent hydrogen,
R^{2b} and R^{2c} each represent methyl,
R⁹ represents ethyl,
R^{3c} and R^{4a} each represent hydrogen or acetyl and
R¹⁰ represents alkyl;
(3) X^{d} represents —C(═O)CR⁸R⁹R¹⁰,
R^{1b}, R^{3b} and R⁸ each represent hydrogen,
R^{2b} and R^{2c} each represent methyl,
R^{3c} and R^{4a} each represent hydrogen or acetyl and
R⁹ and R¹⁰ together with the carbon atom to which they are attached form a cyclohexyl ring;
(4) X^{d} represents unsubstituted —C(═O)phenyl, —C(═O)-4-chlorophenyl or —C(═O)-3-phenoxyphenyl,
R^{1b}, R^{3b}, R^{3c} and R^{4a} each represent hydrogen and
R^{2b} and R^{2c} each represent methyl;
(5) X^{d} represents —C(═O)NR^{6a}R^{7a},
R^{1b} represents hydrogen,
R^{2b} represents hydrogen, methyl or ethyl,
R^{2c} represents methyl or ethyl,
R^{3b} represents hydrogen,
R^{3c} and R^{4a} both represent hydrogen,
R^{6a} represents hydrogen, C₁-C₆-alkyl,
R^{7a} represents alkyl which is optionally mono- or polysubstituted by identical or different substituents or represents -M^{a}-Q^{a}-Z^{a},
M^{a} represents optionally substituted phenylene,
Q^{a} represents a direct bond, C₁-C₄-alkylene, C₁-C₄-alkylenoxy, oxy-C₁-C₄-alkylene, O, S or NR^{12a},
R^{12a} represents hydrogen or C₁-C₆-alkyl,
Z^{a} represents phenyl which is optionally mono- to pentasubstituted by identical or different substituents, where the substituents are selected from the list W¹,
W¹ represents halogen, cyano, nitro, formyl;
in each case straight-chain or branched alkyl, alkoxy, alkylthio, alkylsulphynyl or alkylsulphonyl having in each case 1 to 6 carbon atoms;
in each case straight-chain or branched haloalkyl, haloalkoxy, haloalkylthio, haloalkylsulphynyl or haloalkylsulphonyl having in each case 1 to 6 carbon atoms and 1 to 13 identical or different halogen atoms;
in each case straight-chain or branched akylamino, dialkylamino, alkylcarbonyloxy, alkoxycarbonyl having 1 to 6 carbon atoms in the respective hydrocarbon chains;
in each case doubly attached dioxyalkylene, each of which is optionally mono- to tetrasubstituted by identical or different substituents from the group consisting of fluorine and chlorine and has 1 or 2 carbon atoms.

4. Compositions for controlling unwanted microorganisms, characterized in that they comprise at least one 2-pyridone of the formula (I-k) or 2-pyridinol of the formula (II-b) according to claim 3, in addition to extenders and/or surfactants.

5. Use of 2-pyridones of the formula (I-k) or 2-pyridinols of the formula (II-b) according to claim 3 for controlling unwanted microorganisms.

6. Method for controlling unwanted microorganisms, characterized in that 2-pyridones of the formula (I-k) or 2-pyridinols of the formula (II-b) according to claim 3 are applied to the microorganisms and/or their habitat.
